SEQUENCE LISTING (8 sequences)

SEQ ID NO: 1 - DNA, 20 bp, synthetic construct
  source 1..20: mol_type = other DNA
gtactggattgaccccaacc

SEQ ID NO: 2 - DNA, 20 bp, synthetic construct
  source 1..20: mol_type = other DNA
cgccatactcgaactggaat

SEQ ID NO: 3 - DNA, 20 bp, synthetic construct
  source 1..20: mol_type = other DNA
ccccatctatgagggctatg

SEQ ID NO: 4 - DNA, 20 bp, synthetic construct
  source 1..20: mol_type = other DNA
cagtggccatctcattttca

SEQ ID NO: 5 - DNA, 19 bp, synthetic construct
  source 1..19: mol_type = other DNA
tcgccctgtggatgactga

SEQ ID NO: 6 - DNA, 22 bp, synthetic construct
  source 1..22: mol_type = other DNA
cagagacagccaggagaaatca

SEQ ID NO: 7 - DNA, 20 bp, synthetic construct
  source 1..20: mol_type = other DNA
gtacgctgtgaaggcatcaa

SEQ ID NO: 8 - DNA, 19 bp, synthetic construct
  source 1..19: mol_type = other DNA
gttggtgttcatccgcttg

(19) United States
(12) Patent Application Publication
(10) Pub. No.: US 2026/0185169 A1
(43) Pub. Date: Jul. 2, 2026

(54) MOLECULAR SIGNATURES TO PREDICT LONG-TERM LIVER FIBROSIS PROGRESSION

(71) Applicants: THE BOARD OF REGENTS OF THE UNIVERSITY OF TEXAS SYSTEM, Austin, TX (US); UNIVERSITÉ DE STRASBOURG, Strasbourg (FR); INSERM (INSTITUT NATIONAL DE LA SANTÉ ET DE LA RECHERCHE MÉDICALE), Paris (FR); HÔPITAUX UNIVERSITAIRES DE STRASBOURG (HUS), Strasbourg (FR)

(72) Inventors: Yujin HOSHIDA, Dallas, TX (US); Naoto FUJIWARA, Tsu (JP); Amit SINGAL, Dallas, TX (US); Thomas Fredy BAUMERT, Freiburg (DE)

(73) Assignees: THE BOARD OF REGENTS OF THE UNIVERSITY OF TEXAS SYSTEM, Austin, TX (US); UNIVERSITÉ DE STRASBOURG, Strasbourg (FR); INSERM (INSTITUT NATIONAL DE LA SANTÉ ET DE LA RECHERCHE MÉDICALE), Paris (FR); HÔPITAUX UNIVERSITAIRES DE STRASBOURG (HUS), Strasbourg (FR)

(21) Appl. No.: 19/128,858

(22) PCT Filed: Nov. 10, 2023
(86) PCT No.: PCT/US2023/079339

§ 371 Date: May 9, 2025

Related U.S. Application Data
(60) Provisional application No. 63/383,441, filed on Nov. 11, 2022.
(60) Provisional application No. 63/490,698, filed on Mar. 16, 2023.

(51) Int. Cl. C12Q 1/70; C12Q 1/6883

(52) U.S. Cl. CPC C12Q 1/707; C12Q 1/6883; C12Q 2600/112; C12Q 2600/158

(57) ABSTRACT

Disclosures herein are directed to methods and compositions for predicting high- and low-risk for liver fibrosis progression in patients. Based on the results achieved from the methods and compositions disclosed herein, liver disease patients can be classified into a prognostic risk group, which enables early diagnosis and prevention of liver fibrosis and other lethal complications. Methods and compositions disclosed herein substantially improve the poor prognosis of subjects having or at risk for one or more liver fibrosis.

### CROSS-REFERENCE TO RELATED APPLICATIONS

This application is an International PCT Application claiming priority to U.S. Provisional Application No. 63/383,441, filed Nov. 11, 2022, and U.S. Provisional Application No. 63/490,698, filed Mar. 16, 2023, which are both incorporated herein by reference in their entirety.

### ACKNOWLEDGEMENT OF GOVERNMENT SUPPORT

This invention was made with government support under Grant No. CA233794 awarded by the National Institutes of Health. The government has certain rights in this invention.

### SEQUENCE LISTING

This application contains a Sequence Listing which has been submitted electronically in XML format and is hereby incorporated by reference in its entirety. The XML copy, created Nov. 2, 2023, is 106546-773794_UTSD_4064-PCT.xml and is about 8,000 bytes in size.

### BACKGROUND

### 1. Field

The present inventive concept is directed to methods of determining a fibrosis progression signature FPS and fibrosis progression secretome signature (FPSec) score for use in prediction of risk for developing liver fibrosis and liver fibrosis progression in a subject.

### 2. Discussion of Related Art

The liver is one of the major organs affected by fibrosis due to chronic infection of hepatotropic viruses, e.g., hepatitis B virus (HBV) and hepatitis C virus (HCV), and metabolic disorders, e.g., alcohol-associated liver disease (ALD) and non-alcoholic fatty liver disease/non-alcoholic steatohepatitis (NAFLD/NASH). Cirrhosis is the terminal stage of progressive liver fibrosis, affecting 1% to 2% of the global population and causing 1 million deaths annually worldwide, with >50% increase over the past three decades. Cirrhosis is the major predisposing factor for liver cancer, the fourth leading cause of cancer death worldwide. Given the limited survival benefit and high costs of currently available treatment options at advanced stages of the disease, prevention of fibrosis progression at earlier stages is an urgent unmet need to effectively improve the poor prognosis. Fibrosis progression toward cirrhosis typically takes two to three decades. Therefore, it is practically difficult or infeasible to clinically confirm prognostic benefit of experimental anti-fibrotic therapies. Thus, reliable surrogate biomarkers predictive of long-term fibrosis progression need to be developed for estimating clinically meaningful prognostic benefit of anti-fibrotic therapies within the timeframe of typical therapeutic clinical trials.

### SUMMARY OF THE INVENTION

The present disclosure is based, in part, on the novel finding that determining the abundance of proteins in a biological sample obtained from a subject can be used to generate an FPSec score for use in prediction of liver fibrosis progression in a subject. Accordingly, provided herein are methods and kits for measuring protein abundance of a panel of circulating proteins, determining an FPSec score, and treating high- and low-risk liver fibrosis subjects according to their FPSec score.

Additional aspects of the present disclosure are based, in part, on the novel finding that evaluating a gene expression profile of a biological sample obtained from a sample can be used to generate an FPS score for use in prediction of liver fibrosis progression. Accordingly, provided herein are methods and kits for evaluating gene expression in a patient sample, determining an FPS score, and treating high- and low risk liver fibrosis subjects according to their FPS score.

Aspects of the present disclosure provide for methods of predicting risk for liver fibrosis progression in a subject. In some embodiments, methods of predicting risk for liver fibrosis progression in a subject may comprise determining an FPSec score for the subject, wherein the subject may have or be suspected of having a disease, a condition, or a combination thereof that predisposes the subject to liver fibrosis progression. In some embodiments, methods of predicting risk for liver fibrosis progression in a subject may comprise determining an FPS score for the subject, wherein the subject may have or be suspected of having a disease, a condition, or a combination thereof that predisposes the subject to liver fibrosis.

In some embodiments, methods of predicting risk for liver fibrosis progression in a subject may further comprise a method of obtaining the FPSec score for the subject, wherein the method of obtaining the FPSec score can include any of the following steps: (a) obtaining a sample of blood from the subject; (b) subjecting the sample to a multi-analyte profiling assay for protein quantification of angiogenin, matrix metallopeptidase 7 (MMP-7), insulin like growth factor binding protein 7 (IGFBP-7), protein S (PROS1), vascular cell adhesion molecule 1 (VCAM-1), interleukin 6 (IL-6) C-C motif chemokine ligand 21 (CCL-21); (c) normalizing the protein quantification measurements of angiogenin, MMP-7, IGFBP-7, protein S, VCAM-1, IL-6, and CCL-21 to median fluorescent intensity; and/or, (d) converting the normalized protein quantification measurements of angiogenin, MMP-7, IGFBP-7, protein S, VCAM-1, IL-6, and CCL-21 into an aggregated score, wherein the aggregated score is the FPSec score. In some embodiments, a subject disclosed herein may be predicted to be at low risk for developing long term liver fibrosis if the FPSec score is below a given threshold (e.g., 3). In some aspects, a subject disclosed herein may be predicted to be at high risk for liver fibrosis progression if the FPSec score is higher than a given threshold (e.g., 3).

Other aspects of the present disclosure provide methods of determining an FPSec score for a subject. In some embodiments, methods of determining an FPSec score for a subject may include any of the following steps: (a) obtaining a sample of blood from the subject; (b) subjecting the sample to a multi-analyte profiling assay for protein quantification of angiogenin, matrix metallopeptidase 7 (MMP-7), insulin like growth factor binding protein 7 (IGFBP-7), protein S (PROS1), vascular cell adhesion molecule 1 (VCAM-1), interleukin 6 (IL-6) C-C motif chemokine ligand 21 (CCL-21) (c) normalizing protein quantification measurements angiogenin, MMP-7, IGFBP-7, protein S, VCAM-1, IL-6, and/or CCL-21, to median fluorescent intensity; and/or, (d) converting the normalized protein quantification measurements of angiogenin, MMP-7, IGFBP-7, protein S, VCAM-1, IL-6, and/or CCL-21 into an aggregated score, wherein the aggregated score is the FPSec score. In various aspects, a subject having an FPSec lower than a threshold (e.g., 3) is considered at low risk for liver fibrosis progression. In other aspects, a subject having an FPSec higher than a threshold (e.g., 3) is considered at high risk for liver fibrosis progression.

In some embodiments, methods of predicting risk for liver fibrosis progression in a subject may further comprise a method of obtaining the FPS score for the subject, wherein the method of obtaining the FPS score can include any of the following steps: (a) obtaining a subjecting the sample to a multi-analyte profiling liver biopsy sample from the subject; (b) assay for gene expression of one or more genes, wherein the genes comprise ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9 or any combination thereof, to obtain a gene expression measurement for each of the one or more genes, (c) normalizing the gene expression measurements of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to expression levels of a control set of genes to obtain a gene expression profile of the subject; and (d) converting the gene expression profile of the subject into an FPS score, wherein the FPS score is a numerical value corresponding to a similarity between the gene expression profile of the subject and a high-risk reference gene expression profile or a low-risk reference gene expression profile. In some embodiments, a subject disclosed herein may be predicted to be at low risk for liver fibrosis progression (i.e., long term liver fibrosis) if the FPS score is below a given threshold (e.g., −3.3013). In some aspects, a subject disclosed herein may be predicted to be at high risk for liver fibrosis progression if the FPS score is higher than a given threshold (e.g., +3.3013). In some aspects, a subject disclosed herein may be predicted to be at intermediate risk for liver fibrosis progression if the FPS score is between −1.3013 and +1.3013.

Other aspects of the present disclosure provide methods of determining an FPS score for a subject. In some embodiments, methods of determining an FPS score for a subject may include any of the following steps: (a) obtaining a liver biopsy sample from the subject; (b) subjecting the sample to a multi-analyte profiling assay for gene expression of one or more genes, wherein the genes comprise ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9 or any combination thereof, to obtain a gene expression measurement for each of the one or more genes, (c) normalizing the gene expression measurements of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to expression levels of a control set of genes to obtain a gene expression profile of the subject; and, (d) converting the gene expression profile of the subject into an FPS score, wherein the FPS score is a numerical value corresponding to a similarity between the gene expression profile of the subject and a high-risk reference gene expression profile or a low-risk reference gene expression profile. In various aspects, a subject having an FPS lower than a threshold (e.g., −3.3013) is considered at low risk for liver fibrosis progression. In other aspects, a subject having an FPSec higher than a threshold (e.g., +3.3013) is considered at high risk for liver fibrosis progression. In various aspects, a subject having an FPS score between −1.3013 and +1.3013 is considered at intermediate risk of liver fibrosis progression.

In any of the preceding or foregoing embodiments, the subject of any of the methods disclosed herein may be having or may be suspected of having a disease, a condition, or a combination thereof that predisposes the subject to liver fibrosis. In some aspects, liver fibrosis is a long-term liver fibrosis. In some aspects, a disease, a condition, or a combination thereof that predisposes the subject to liver fibrosis may be chronic infection of hepatitis B virus (HBV), chronic infection of hepatitis C virus (HCV), non-alcoholic fatty liver disease (NAFLD), non-alcoholic steatohepatitis (NASH), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

In any of the preceding or foregoing embodiments, the methods may further comprise diagnosing liver fibrosis in the subject. In various aspects, diagnosing liver fibrosis may comprise performing a liver biopsy, one or more blood tests to assess liver function, computed tomography, magnetic resonance imaging, or any combination thereof. In some embodiments, one or more blood tests performed to assess liver function may comprise measuring alanine transaminase (ALT), aspartate transaminase (AST), alkaline phosphatase (ALP), albumin, bilirubin, gamma-glutamyltransferase (GGT), L-lactate dehydrogenase (LD), prothrombin time (PT), or any combination thereof.

In any of the preceding or foregoing embodiments, methods disclosed herein may further include administering one or more treatments of liver fibrosis to the subject. In some aspects, the one or more treatments of liver fibrosis may comprise an anti-fibrotic therapy. In some aspects, an anti-fibrotic therapy for use herein may be administration of one or more drugs to the subject, wherein the drugs can be selected from galunisertib, erlotinib, AM095, bortezomib, pioglitazone, metformin, epigallocatechin gallate (EGCG), I-BET 151, JQ1, captopril, nizatidine; MG-132; and cenicriviroc.

Still other aspects of the present disclosure provide diagnostic kits for determining an FPSec score of a subject. In some embodiments, kits disclosed herein may contain one or more reagents for use in a multi-analyte profiling assay. In some embodiments, kits disclosed herein may contain one or more reagents for use in a multi-analyte profiling assay such as beads labeled with antibodies to angiogenin, matrix metallopeptidase 7 (MMP-7), insulin like growth factor binding protein 7 (IGFBP-7), protein S (PROS1), vascular cell adhesion molecule 1 (VCAM-1), interleukin 6 (IL-6), and/or C-C motif chemokine ligand 21 (CCL-21).

Still other aspects of the present disclosure provide diagnostic kits for determining an FPS score of a subject. In some embodiments, kits disclosed herein may contain one or more reagents for use in a multi-analyte profiling assay. In some embodiments, kits disclosed herein may contain one or more reagents for use in a multi-analyte profiling assay such as one or more nucleic acid probes labeled with color-coded microbeads to mRNA transcribed from one or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9.

Other aspects of the present disclosure provide methods of treating liver fibrosis in a subject at high risk for liver fibrosis progression. In some embodiments, methods herein of treating liver fibrosis in a subject at high risk for liver fibrosis progression can include any of the following steps: (a) determining if the subject is at high risk for developing liver fibrosis by (i) obtaining a sample of blood from the subject; (ii) determining the protein levels of at least two liver disease biomarkers wherein, one of the at least two liver disease biomarkers is selected from vascular cell adhesion molecule 1 (VCAM-1), insulin-like growth factor-binding protein 7 (IGFBP-7), matrix metallopeptidase 7 (MMP-7), interleukin-6 (IL-6), and C-C motif chemokine ligand 21 (CCL-21); and the other one of the at least two liver disease biomarkers is selected from angiogenin and protein S; (iii) determining that the subject is at high risk for developing liver fibrosis progression if the one of the at least two liver disease biomarkers selected from vascular cell adhesion molecule 1 (VCAM-1), insulin-like growth factor-binding protein 7 (IGFBP-7), matrix metallopeptidase 7 (MMP-7), interleukin-6 (IL-6), and C-C motif chemokine ligand 21 (CCL-21) has a higher protein expression compared to a control, and the other one of the at least two liver disease biomarkers selected from angiogenin and protein S (PROS1), has a lower protein expression compared to a control, wherein the control is a sample of blood from a subject known to not have any liver disease; and/or (b) administering one or more treatments of liver fibrosis to the subject determined to be at high risk for liver fibrosis progression.

In some embodiments, protein levels of angiogenin, matrix metallopeptidase 7 (MMP-7), insulin like growth factor binding protein 7 (IGFBP-7), protein S (PROS1), vascular cell adhesion molecule 1 (VCAM-1), interleukin 6 (IL-6) C-C motif chemokine ligand 21 (CCL-21) may be determined according to the methods disclosed herein, wherein the subject is at high risk for liver fibrosis progression if any one of MMP-7, IGFBP-7, VCAM-1, IL-6, and CCL-21 has a higher protein expression compared to a control and any one of angiogenin and protein S, has a lower protein expression compared to a control. In some embodiments, the protein levels of MMP-7, IGFBP-7, VCAM-1, IL-6, CCL-21, angiogenin and/or protein S, may be determined according to the methods disclosed herein, wherein the subject is at high risk for liver fibrosis progression if MMP-7, IGFBP-7, VCAM-1, IL-6, and/or CCL-21, have a higher protein expression compared to a control and angiogenin and/or protein S, have a lower protein expression compared to a control.

In some embodiments, the level of at least two liver disease biomarkers according to the methods disclosed herein may be determined by one or more of the following: Western blotting, enzyme-linked immunosorbent assay (ELISA), multi-analyte profiling assay, mass spectrometry, HPLC, flow cytometry, fluorescence-activated cell sorting (FACS), liquid chromatography-mass spectrometry (LC/MS), immunoelectrophoresis, translation complex profile sequencing (TCP-seq), protein microarray, protein chip, capture arrays, reverse phase protein microarray (RPPA), two-dimensional gel electrophoresis or (2D-PAGE), functional protein microarrays, electrospray ionization (ESI), and matrix-assisted laser desorption/ionization (MALDI). In some aspects, the level of at least two liver disease biomarkers may be determined by ELISA or multi-analyte profiling assay.

Other aspects of the present disclosure provide methods of treating liver fibrosis in a subject at high risk for liver fibrosis progression. In some embodiments, methods herein of treating liver fibrosis in a subject at high risk for liver fibrosis progression can include any of the following steps: (a) determining if the subject is at high risk for liver fibrosis progression by (i) obtaining a liver biopsy sample from the subject; (ii) subjecting the liver biopsy sample to a multi-analyte profiling assay for gene expression of one or more genes, wherein the genes comprise ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to obtain a gene expression measurement of each of the one or more genes; (iii) normalizing the gene expression measurements of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to expression levels of a control set of genes to obtain a gene expression profile of the subject; (iv) converting the gene expression profile of the subject into an FPS score, wherein the FPS score is a numerical value corresponding to a similarity between the gene expression profile of the subject and a high-risk reference gene expression profile or a low-risk reference gene expression profile; and (v) determining that the subject is at high risk for liver fibrosis progression if the FPS score is greater than +1.3013 and (b) administering one or more treatments of liver fibrosis to the subject determined to be at high risk for developing liver fibrosis. In various aspects, the one or more treatments of liver fibrosis may comprise an anti-fibrotic therapy such as galunisertib, erlotinib, AM095, bortezomib, pioglitazone, metformin, epigallocatechin gallate (EGCG), I-BET 151, JQ1, captopril, and nizatidine (Selleck Chemicals); MG-132; or cenicriviroc.

In any of the foregoing aspects, the gene expression level of one or more genes is determined by one or more methods selected the group consisting of microarrays, high-density expression array, DNA microarray, polymerase chain reaction (PCR), reverse transcriptase PCR (RT-PCR), real-time quantitative reverse transcription PCR (qRT-PCR), digital droplet PCR (ddPCR), serial analysis of gene expression (SAGE), Spotted cDNA arrays, GeneChip, spotted oligo arrays, bead arrays, RNA Seq, tiling array, northern blotting, hybridization microarray, in situ hybridization, or any combination thereof.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein. Embodiments of the present inventive concept are illustrated by way of example in which like reference numerals indicate similar elements.

FIG. 1 illustrates an aspect of the subject matter in accordance with one embodiment and depicts a study design of Prognostic Liver Signature (PLS) validation and Fibrosis Progression Signature (FPS) derivation and validation.

FIG. 2 depicts the validation of Prognostic Liver Signature (PLS) for 5-year fibrosis progression. FIG. 2A depicts expression pattern of the PLS genes. FIG. 2B depicts odds ratios (blue squares) and 95% CI (horizontal line) for high-risk PLS and clinical prognostic variables in multivariable logistic regression. FIG. 2C depicts AUROC curve of the PLS-based prognostic prediction for 5-year fibrosis progression in the PLS validation set 1 (left) and 2 (right).

FIG. 3 illustrates an aspect of the subject matter in accordance with one embodiment and depicts derivation and validation of a Fibrosis Progression Signature (FPS). FIG. 3A shows expression pattern of the FPS genes in the FPS derivation sets 1 to 4. FIG. 3B shows association of each FPS member gene with NAFLD fibrosis stage (n=71) and histological severity (n=72; “severe” and “mild” indicate F3-4 and F0-1 fibrosis, respectively), HCV cirrhosis (n=216) and HBV chronic hepatitis/cirrhosis (n=199) prognosis (see Table 8). FIG. 3C shows expression pattern of FPS and clinical annotations in the FPS validation set 1 (n=78). FIG. 3D shows odds ratios (blue squares) and 95% CI (horizontal line) for high-risk FPS and clinical prognostic variable in multivariable logistic regression. FIG. 3E depicts AUROC of the FPS-based prognostic prediction for fibrosis progression (left) and no fibrosis regression (right). FIG. 3F depicts correlation of the time-interval-adjusted change in FPS-based prognostic risk level (measured by combined enrichment score [CES]) with the changes in histological, biochemical, and clinical variables between the two time points of liver biopsy. * Obesity is defined by the WHO guidelines (i.e., BMI >30 kg/m²)⁴⁶ for the U.S. cohorts and the Asian-Pacific guidelines (i.e., BMI >25 kg/m²)⁴⁷ for the Japanese cohorts, considering race/ethnicity-specific impact of BMI on metabolic disease and prognosis.

FIG. 4 illustrates an aspect of the subject matter in accordance with one embodiment and shows that BCL2 is an FPS-associated anti-fibrosis target in clinical fibrotic liver tissues. FIG. 4A depicts a co-expression gene network defined in the FPS derivation set 1 to 4. Hub genes are indicated with larger nodes. Synthesized association with time to fibrosis progression (Fisher's inverse chi-square statistic) in the FPS derivation set 1 and 2 is shown by red (poor outcome) to blue (good outcome) color scale. FIG. 4B shows dysregulation of BCL2-co-expressed gene module, apoptosis-related gene set, and hepatic stellate cell (HSC)-related gene signatures. FIG. 4C shows reduced expression of COL1A1, ACTA2 (encoding α-smooth muscle actin [SMA]), and BCL2 with MG-132 in LX-2 and TWNT-4 cells, and organotypic ex vivo culture of clinical fibrotic precision-cut liver slice (PCLS) tissues from 2 patients (ev144 [HCV (Hepatitis C Virus), F1], ev145 [NAFLD (Non-Alcoholic Fatty Liver Disease), F2]). All assays were performed in triplicates. Green dotted line indicates expression level of the DMSO-treated control. FIG. 4D shows the difference in number of cells positive for cleaved caspase-3 per unit area between replicated PCLS tissues cultured with MG-132 or DMSO from five patients. Paired tissues from the same patient are connected with a line. Wilcoxon signed-rank test p-value is shown. FIG. 4E shows immunohistochemical staining of α-SMA and cleaved caspase-3 in MG-132-treated (upper panel) and DMSO-treated (lower panel) clinical PCLS tissue (ev145). Scale bars indicate 50 μm and 25 μm for upper and lower panels, respectively. FIG. 4F shows immunofluorescence staining of an HSC marker, glial fibrillary acidic protein (GFAP) (red), and cleaved caspase-3 (green) showing their co-localization (yellow) in MG-132-treated (upper panel) and DMSO-treated (lower panel) clinical PCLS tissue (ev145). Scale bars indicate 100 μm. FIG. 4G shows modulation of FPS high- and low-risk genes measured by gene set enrichment analysis in the clinical PCLS tissues. NES: normalized enrichment score. FDR: false discovery rate.

FIG. 5 illustrates an aspect of the subject matter in accordance with one embodiment and depicts FPS-based systematic evaluation of anti-fibrotic agents in ex vivo culture of clinical PCLS tissues. FIG. 5A shows patient-level modulation of FPS genes by a panel of anti-fibrotic agents in organotypic ex vivo culture of PCLS tissues in FPS validation set 2 (Table 1B). Patients are ordered by favorable modulation of FPS measured by CES from left to right. FIG. 5B shows drug-level modulation of FPS to depict shared and unique target FPS genes across the tested anti-fibrotic agents. Phenotypic association of CES and differential gene expression were tested by Wilcoxon rank-sum test (when ≥2 samples were available in each group) and paired t-test, respectively. FIG. 5C shows computationally inferred joint effect of combination of the tested anti-fibrotic agents. FIG. 5D shows complementary targeting of FPS genes by combining EGCG with bortezomib (left) or MG-132 (right). FIG. 5E shows validation of the inferred joint effect of the combination therapies profiled by the liver fibrosis gene panel in ex vivo culture of a clinical fibrotic PCLS tissue. FIG. 5F shows validation of the inferred joint effect of the combination therapies profiled by the liver fibrosis gene panel in in vitro culture of a patient-derived liver spheroid. FIG. 5G shows In vitro pharmacological FPS modulation in a cell culture system.

FIG. 6 illustrates an aspect of the subject matter in accordance with one embodiment and depicts modulation of FPS and molecular pathways by cenicriviroc in a phase II clinical trial. FIG. 6A shows F-stage change and FPS modulation in cenicriviroc- and placebo-treated NASH patients. FIG. 6B shows a correlation between FPS modulation measured by CES and F-stage change. FIG. 6C shows AUROC for association between CES and 1-year histological fibrosis change. FIG. 6D shows modulation of FPS member genes with the 1-year cenicriviroc treatment in patients with (yes) or without (no) F-stage improvement. GSEI: gene set enrichment index. FIG. 6E shows modulation of molecular pathways with the 1-year cenicriviroc treatment in patients with (yes) or without (no) F-stage improvement. GSEI: gene set enrichment index. FIG. 6F shows modulation of nuclear receptor signaling pathways with the 1-year cenicriviroc treatment in patients with (yes) or without (no) F-stage improvement. GSEI: gene set enrichment index. FIG. 6G shows validation of the inferred joint effect of the combination therapies profiled by the liver fibrosis gene panel in in vitro culture of a patient-derived liver spheroid.

FIG. 7 illustrates an aspect of the subject matter in accordance with one embodiment and depicts derivation and validation of Fibrosis Progression Secretome signature (FPSec). FIG. 7A shows correlation of prognostic prediction between tissue-transcriptome-based FPS and serum-protein-based FPSec in a cohort of Japanese cirrhosis patients with mixed etiologies (n=79) from Fujiwara N et al., (“A Blood-Based Prognostic Liver Secretome Signature Predicts Long-term Risk of Hepatic Decompensation in Cirrhosis'. Clin Gastroenterol Hepatol 2021). FIG. 7B shows validation of the FPSec in an independent cohort of American patients with compensated cirrhosis (n=122) for development of incident hepatic decompensation (the hazard proportionality test P=0.17).

FIG. 8 illustrates an aspect of the subject matter in accordance with one embodiment and depicts the computational derivation of Fibrosis Progression Signature (FPS). FIG. 8A shows a computational derivation of Fibrosis Progression Signature (FPS). To define the FPS genes, association of each gene's expression with time to fibrosis progression was synthesized in the FPS derivation sets 1 and 2 (upper panel). In parallel, shared gene co-expression between the FPS derivation sets 1-2 (HCV etiology) and the sets 3-4 (NAFLD etiology) were integrated to identify etiology-agnostic co-expressed genes (lower panel). FIG. 8B shows selection of FPS genes base on association with time to fibrosis progression (y-axis) and co-expression irrespective of the liver disease etiology (x-axis). FIG. 8C shows F-stage progression, PLS risk prediction, and FPS risk prediction across the patients in the FPS derivation sets 1˜4. FIG. 8D shows PLS/FPS risk predictions and F-stage progression in the FPS derivation set 1. FIG. 8E shows PLS/FPS risk predictions and F-stage progression in the FPS derivation set 2. FIG. 8F illustrates an aspect of the subject matter in accordance with one embodiment shows the relationship of FPS risk predictions between the baseline and follow-up biopsies in the FPS validation set 1. FIG. 8G shows the correlation of FPS risk prediction with presence of >F2 fibrosis in the FPS validation set 1 (logistic regression OR=5.00, 95% CI=0.51-48.46, P=0.16; AUROC=0.58) .

FIG. 9 illustrates an aspect of the subject matter in accordance with one embodiment and depicts molecular dysregulations in hepatic cell types in fibrotic mouse liver. Dysregulation of BCL2-co-expressed gene module, apoptosis-related gene set, and hepatic stellate cell (HSC)-related gene signatures in each cell type in single-cell RNA-Seq of fibrotic mouse livers.

FIG. 10 illustrates an aspect of the subject matter in accordance with one embodiment and depicts modulation of the FPS member genes by the mono and combination therapies in a clinical fibrotic PCLS tissue. The high-risk FPS genes were more broadly suppressed with the combinations compared to mono-therapies.

FIG. 11 illustrates an aspect of the subject matter in accordance with one embodiment and depicts FPS-based risk prediction in each individual patient over the course of clinical follow-up without therapeutic interventions (upper panel; FPS validation set 1) and 1-year treatment with cenicriviroc or placebo (lower panel; FPS validation set 3).

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, in part, on the novel finding that determining a gene expression profile or protein abundance levels in in a biological sample obtained from a subject can be used to generate an FPS score and/or an FPSec score for use in prediction of development and progression of liver fibrosis (e.g., long-term liver fibrosis) in the subject. Accordingly, provided herein are methods for determining gene expression in a tissue and/or measuring protein abundance of a panel of circulating proteins, determining an FPS and/or FPSec score, and treating patients at high or low risk of developing liver fibrosis according to their FPS and/or FPSec score. Kits used in practicing the methods disclosed herein are also provided in the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or +1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

“Biomarker” as used herein refers to any biological molecules (e.g., nucleic acids, genes, peptides, proteins, lipids, hormones, metabolites, and the like) that, singularly or collectively, reflect the current or predict future state of a biological system. Thus, as used herein, the presence or concentration of one or more biomarkers can be detected and correlated with a known condition, such as a disease state. In some aspects, detecting the presence and/or concentration of one or more biomarkers herein may be an indication of a liver cancer risk in a subject. In some other aspects, detecting the presence and/or concentration of one or more biomarkers herein may be used in treating and/or preventing a liver cancer in a subject.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “liver fibrosis” refers to the excessive accumulation of extracellular matrix proteins in liver tissue and is a byproduct of most chronic liver diseases. It can be progressive with advanced liver fibrosis resulting in cirrhosis, liver failure, and portal hypertension and ultimately the need for liver transplantation. As used herein, the term “long-term liver fibrosis” is synonymous and used interchangeably with the term “liver fibrosis progression” can ultimately progress to cirrhosis.

It should also be understood that, unless clearly indicated to the contrary, in any methods claimed herein that include more than one step or act, the order of the steps or acts of the method is not necessarily limited to the order in which the steps or acts of the method are recited.

II. Methods of Determining a Fibrosis Progression Signature (FPS) Score and/or a Fibrosis Progression Secretome Signature (FPSec) Score

In general, methods disclosed herein include determining an FPS and/or an FPSec score for a subject, wherein the determined FPS score and/or FPSec score can be used to predict the risk for developing liver fibrosis and/or risk of liver fibrosis progression in the subject, the prognostic outcome for a subject having or suspected of having liver fibrosis, and/or providing a suitable treatment regimen to the subject. Standard procedures for diagnosing and monitoring liver fibrosis are difficult to apply when liver fibrosis is minor or incipient. However liver fibrosis becomes significantly harder to treat as it matures and progresses. Therefore, the present disclosure provides novel methods of tracking the risk in a subject for long term liver fibrosis progression by determining an FPS or FPSec score of the subject.

As used herein, a suitable subject includes a mammal, a human, a livestock animal, a companion animal, a lab animal, or a zoological animal. In some embodiments, a subject may be a rodent, e.g., a mouse, a rat, a guinea pig, etc. In other embodiments, a subject may be a livestock animal. Non-limiting examples of suitable livestock animals may include pigs, cows, horses, goats, sheep, llamas and alpacas. In yet other embodiments, a subject may be a companion animal. Non-limiting examples of companion animals may include pets such as dogs, cats, rabbits, and birds. In yet other embodiments, a subject may be a zoological animal. As used herein, a “zoological animal” refers to an animal that may be found in a zoo. Such animals may include non-human primates, large cats, wolves, and bears. In other embodiments, the animal is a laboratory animal. Non-limiting examples of a laboratory animal may include rodents, canines, felines, and non-human primates. In some embodiments, the animal is a rodent. Non-limiting examples of rodents may include mice, rats, guinea pigs, etc. In preferred embodiments, the subject is a human.

In some embodiments, a suitable subject for the methods herein may have or be suspected of having liver fibrosis. In some embodiments, a suitable subject for the methods herein may have or be suspected of having a liver disease or condition that predisposes a subject to liver fibrosis. For example, a liver disease or condition that may predispose the subject to liver fibrosis may be chronic infection of hepatitis B virus (HBV), chronic infection of hepatitis C virus (HCV), non-alcoholic fatty liver disease (NAFLD), non-alcoholic steatohepatitis (NASH), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof. In some embodiments, a suitable subject for the methods herein may have or be suspected of having one or more injuries to the liver that may predispose a subject to liver fibrosis.

In some embodiments, a suitable subject for the methods herein may present with at least one clinical symptom associated with liver fibrosis. Non-limiting examples of clinical symptoms associated with liver fibrosis may include mild to moderate upper abdominal pain, weight loss, early satiety, jaundice, edema especially in the lower extremities, nausea and weakness

In some embodiments, an FPS score and/or an FPSec score may be determined as disclosed herein from at least one sample collected from a subject. In some aspects, at least one sample can be obtained from a subject who has not been diagnosed with a liver disease or condition associated with liver fibrosis. In some aspects, at least one sample can be obtained from a subject who has not been diagnosed with a liver disease or condition associated with liver fibrosis but is suspected of having the liver disease or condition. In some other aspects, at least one sample can be obtained from a subject who has been diagnosed with a liver disease or condition associated with liver fibrosis. In some aspects, at least one sample can be obtained from a subject who may have or be suspected of having one or more injuries to the liver that may predispose a subject to liver fibrosis.

In some embodiments, an FPS score may be determined by obtaining a gene expression profile from a sample collected from a subject. As used herein, the term “gene expression profile” refers to a pattern of genes expressed in a sample at the transcription level. Non-limiting examples of methods of measuring gene expression in a sample suitable for use herein include digital transcript counting, high-density expression array, DNA microarray, polymerase chain reaction (PCR), reverse transcriptase PCR (RT-PCR), real-time quantitative reverse transcription PCR (qRT-PCR), digital droplet PCR (ddPCR), serial analysis of gene expression (SAGE), Spotted cDNA arrays, GeneChip, spotted oligo arrays, bead arrays, RNA Seq, tiling array, northern blotting, hybridization microarray, in situ hybridization, or any combination thereof. In some aspects, a gene expression profile as disclosed herein can be obtained by any known or future method suitable to assess gene expression.

In some embodiments, an FPSec score may be determined by obtaining a protein expression profile from a sample collected from a subject. As used herein, the term “protein expression profile” refers to a pattern of proteins expressed in a sample collected from the subject. Non-limiting examples of methods of measuring protein expression in a sample suitable for use herein include Western blotting, enzyme-linked immunosorbent assay (ELISA), multi-analyte profiling (xMAP), mass spectrometry, HPLC, flow cytometry, fluorescence-activated cell sorting (FACS), liquid chromatography-mass spectrometry (LC/MS), immunoelectrophoresis, translation complex profile sequencing (TCP-seq), protein microarray, protein chip, capture arrays, reverse phase protein microarray (RPPA), two-dimensional gel electrophoresis or (2D-PAGE), functional protein microarrays, electrospray ionization (ESI), matrix-assisted laser desorption/ionization (MALDI), or a combination thereof. In some aspects, a protein expression profile as disclosed herein can be obtained by any known or future method suitable to assess protein expression.

In some embodiments, a sample obtained from a subject for determination of an FPS score and/or an FPSec score as disclosed in the methods herein may be a tissue sample, a blood sample, a plasma sample, a hair sample, venous tissues, cartilage, a sperm sample, a skin sample, an amniotic fluid sample, a buccal sample, saliva, urine, serum, sputum, bone marrow or a combination thereof. In some aspects, a sample obtained from a subject for determination of an FPS score and/or an FPSec score as disclosed herein may be a liver tissue sample (e.g., a biopsy).

In some embodiments, a sample obtained from a subject for determination of an FPSec score as disclosed herein may be a blood, serum and/or plasma sample. In some aspects, a liver sample for use in the methods herein can be liver proteins isolated from a blood sample collected from any of the subjects disclosed herein. In some aspects, a sample obtained from a subject for determination of an FPSec score as disclosed herein may be serum.

In some embodiments, a sample obtained from a subject for determination of an FPS score as disclosed herein may be a liver tissue sample (e.g., a biopsy). Non-limiting methods suitable for use herein to collect liver tissue include collection by fine needle aspirate, by removal of pleural or peritoneal fluid, and by excisional biopsy. In some aspects, a liver sample can include a biopsy from a single site in the liver, a biopsy from at least one tissue in liver and/or at least one tissue in contact with the liver can be from about 10 mg about 50 mg (e.g., about 10 mg, 15 mg, 20 mg, 25 mg, 30 mg, 35 mg, 40 mg, 45 mg, 50 mg) of tissue per sample.

In some aspects, a sample obtained from for determination of an FPS score or FPSec score as disclosed herein may be stored at about 25° C. to about −80° C. for up to about 1 day to about 2 years, about 1 week to about 1 year, or about 1 month to about 6 months. In other aspects, a sample obtained from a subject may be immediately processed to obtain a protein expression profile as disclosed herein. In some other aspects, a sample obtained from a subject may be processed to obtain a protein expression profile as disclosed herein. Non-limiting examples of sample preparation methods can be found in art, for example in Gallagher & Wiley, (2012). CURRENT PROTOCOLS ESSENTIAL LABORATORY TECHNIQUES. Hoboken, N.J: Wiley-Blackwell, the disclosures of which are incorporated herein.

### (a) Fibrosis Progression Signature (FPS)

In some embodiments, a sample obtained from a subject for determination of an FPS score as disclosed herein consists of a gene expression profile. As used herein, a gene expression profile comprises a pattern of genes expressed in a sample at the transcription level. Non-limiting examples of methods of measuring gene expression in a sample suitable for use herein include high-density expression array, DNA microarray, polymerase chain reaction (PCR), reverse transcriptase PCR (RT-PCR), real-time quantitative reverse transcription PCR (qRT-PCR), digital droplet PCR (ddPCR), serial analysis of gene expression (SAGE), Spotted cDNA arrays, GeneChip, spotted oligo arrays, bead arrays, RNA Seq, tiling array, northern blotting, hybridization microarray, in situ hybridization, digital transcript counting, or any combination thereof. In some aspects, a gene expression profile as disclosed herein can be obtained by any known or future method suitable to assess gene expression. In some embodiments an FPS score as disclosed herein can be determined from a gene expression profile of a liver sample. In some embodiments, an FPS score as disclosed herein can be determined from a gene expression profile expressed by the liver wherein the gene expression profile is comprised of a panel of genes associated with the risk of developing progressive liver fibrosis. In some aspects, a computational biology approach may be applied to identify a gene expression profile associated with the risk of developing progressive liver fibrosis. For example, ranked prioritized genes can be tested for their association with liver fibrosis against a plurality of matched control gene set. Covariates can be adjusted to identify a set of circulating proteins enriched for liver fibrosis (e.g., p<0.001). One or more regression models may be applied to a set of differentially expressed genes to further select for genes that are relevant to liver fibrosis or its progression and the risk associated thereof. In some embodiments, an enriched group of genes for assessing liver fibrosis risk may be a panel of genes that make up a gene expression profile as disclosed herein. In some embodiments, computational approaches exemplified herein can identify panel of genes for prognostic prediction of liver fibrosis risk. As used herein, a “panel of genes” refers to one or more genes whose differential expression (i.e., over-expression or under-expression) is predictive of the risk for developing a pathological condition and/or having a pathological condition. In some embodiments, computational approaches exemplified herein can identify a panel of genes for prognostic prediction of liver fibrosis risk, wherein the panel of genes can be referred to as an FPS.

In some embodiments, an FPS for liver fibrosis progression risk assessment may comprise a combination of one or more genes selected from: ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In various aspects, an FPS for liver fibrosis progression risk assessment may comprise a combination of one or more genes selected from ANXA1, AEBP1, FBN1, IER3, CCL21, CXCR4, KRT7, IGFBP6, FILIP1L LOXL2, BCL2, SLC71, DDR1, and NTS. In other aspects, an FPS for liver fibrosis progression risk assessment may comprise a combination of one or more genes selected from PMM1, NAAA, TTR, PON3, HAAO, and F9.

In some embodiments, an FPS for liver fibrosis progression risk assessment may comprise a combination of two or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of two or more genes selected from ANXA1, AEBP1, FBN1, IER3, CCL21, CXCR4, KRT7, IGFBP6, FILIP1L LOXL2, BCL2, SLC71, DDR1, and NTS. In some embodiments, an FPS for liver fibrosis progression risk assessment may comprise a combination of two or more genes selected from PMM1, NAAA, TTR, PON3, HAAO, and F9.

In some embodiments, an FPS for liver fibrosis progression risk assessment may comprise a combination of three or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of three or more genes selected from ANXA1, AEBP1, FBN1, IER3, CCL21, CXCR4, KRT7, IGFBP6, FILIP1L LOXL2, BCL2, SLC71, DDR1, and NTS. In some embodiments, an FPS for liver fibrosis progression risk assessment may comprise a combination of three or more genes selected from PMM1, NAAA, TTR, PON3, HAAO, and F9.

In some embodiments, an FPS for liver fibrosis progression risk assessment may comprise a combination of four or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of four or more genes selected from ANXA1, AEBP1, FBN1, IER3, CCL21, CXCR4, KRT7, IGFBP6, FILIP1L LOXL2, BCL2, SLC71, DDR1, and NTS. In some embodiments, an FPS for liver fibrosis progression risk assessment may comprise a combination of four or more genes selected from PMM1, NAAA, TTR, PON3, HAAO, and F9.

In some embodiments, an FPS for liver fibrosis progression risk assessment may comprise a combination of five or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of five or more genes selected from ANXA1, AEBP1, FBN1, IER3, CCL21, CXCR4, KRT7, IGFBP6, FILIP1L LOXL2, BCL2, SLC71, DDR1, and NTS. In some embodiments, an FPS for liver fibrosis progression risk assessment may comprise a combination of five or more genes selected from PMM1, NAAA, TTR, PON3, HAAO, and F9.

In some embodiments, an FPS for liver fibrosis progression risk assessment may comprise a combination of six or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of six or more genes selected from ANXA1, AEBP1, FBN1, IER3, CCL21, CXCR4, KRT7, IGFBP6, FILIP1L LOXL2, BCL2, SLC71, DDR1, and NTS. In some embodiments, an FPS for liver fibrosis progression risk assessment may comprise a combination of six genes selected from PMM1, NAAA, TTR, PON3, HAAO, and F9.

In some embodiments, an FPS for liver fibrosis progression risk assessment may comprise a combination of seven or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of seven or more genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of eight or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of eight or more genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of nine or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of nine or more genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of ten or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of ten or more genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of eleven or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of eleven or more genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of twelve or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of twelve or more genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of thirteen or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of thirteen or more genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of fourteen or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of fourteen genes selected from ANXA1, AEBP1, FBN1, IER3, CCL21, CXCR4, KRT7, IGFBP6, FILIP1L LOXL2, BCL2, SLC71, DDR1, and NTS.

In some embodiments, an FPS for liver fibrosis progression risk assessment may comprise a combination of fifteen or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of sixteen or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of seventeen or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of eighteen or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of nineteen or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9. In some embodiments, an FPS for liver fibrosis progression risk assessment may comprise a combination of twenty genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9.

In some embodiments, an FPS for liver fibrosis risk assessment may comprise a combination of one or more genes wherein at least one of the genes is a high-risk-associated gene. In some embodiments, an FPS for liver fibrosis risk assessment may comprise a combination of one or more high-risk-associated genes selected from ANXA1, AEBP1, FBN1, IER3, CCL21, CXCR4, KRT7, IGFBP6, FILIP1L LOXL2, BCL2, SLC71, DDR1, and NTS. In some embodiments, an FPS for liver fibrosis risk assessment may comprise a combination of one or more genes wherein at least one of the genes is a low-risk-associated gene. In some embodiments, an FPS for liver fibrosis risk assessment may comprise a combination of one or more low-risk associated genes selected from: PMM1, NAAA, TTR, PON3, HAAO, and F9. In some embodiments, an FPS for liver fibrosis risk assessment may comprise a combination of one or more high-risk-associated genes of ANXA1, AEBP1, FBN1, IER3, CCL21, CXCR4, KRT7, IGFBP6, FILIP1L LOXL2, BCL2, SLC71, DDR1, NTS or any combination thereof and one or more low-risk-associated genes of PMM1, NAAA, TTR, PON3, HAAO, F9, or any combination thereof.

### (b) Fibrosis Progression Secretome Signature (FPSec)

In some embodiments, a sample obtained from a subject for determination of an FPSec score as disclosed herein consists of a secretome. As used herein, a “secretome” refers to a panel of proteins expressed by an organism and secreted into the extracellular space. In some embodiments an FPSec score as disclosed herein can be determined from a secretome expressed by the liver and secreted into the extracellular space. In some embodiments, an FPSec score as disclosed herein can be determined from a secretome expressed by the liver wherein the secretome is comprised of a panel of proteins associated with the risk of developing a liver cancer. In some aspects, a computational biology approach may be applied to identify a secretome associated with the risk of developing progressive liver fibrosis. For example, ranked prioritized circulating proteins can be tested for their association with liver fibrosis against a plurality of matched control gene set. Covariates can be adjusted to identify a set of circulating proteins enriched for liver fibrosis (e.g., p<0.001). One or more regression models may be applied to the enriched circulating proteins set to further select for proteins that are relevant to liver fibrosis or its progression and the risk associated thereof. In some embodiments, an enriched group of circulating proteins for assessing liver fibrosis risk may be a panel of proteins that make up a secretome as disclosed herein. In some embodiments, computational approaches exemplified herein can identify panel of proteins for prognostic prediction of liver fibrosis risk. As used herein, a “panel of proteins” refers to one or more proteins that are predictive of the risk for developing a pathological condition and/or having a pathological condition. In some embodiments, computational approaches exemplified herein can identify a panel of circulating proteins for prognostic prediction of liver fibrosis risk, wherein the panel of circulating proteins can be referred to as a serum-protein-based FPSec.

In some embodiments, an FPSec for liver fibrosis progression risk assessment may comprise a combination of one or more circulating proteins selected from: vascular cell adhesion molecule 1 (VCAM-1), insulin-like growth factor-binding protein 7 (IGFBP-7), matrix metallopeptidase 7 (MMP-7), interleukin-6 (IL-6), C-C motif chemokine ligand 21 (CCL-21), angiogenin, protein S, or any combination thereof. In some embodiments, an FPSec for liver fibrosis risk assessment may comprise a combination of one or more circulating proteins encoded by the genes of VCAM1, IL6, MMP7, CCL21, IGFBP7, ANG, and PROS1,

In some embodiments, an FPSec for liver fibrosis risk assessment may comprise a combination of one or more circulating proteins of MMP-7, VCAM-1, IGFBP-7, IL-6, CCL-21, protein S, angiogenin or any combination thereof. In some embodiments, an FPSec for liver fibrosis risk assessment may comprise a combination of three or more circulating proteins of MMP-7, VCAM-1, IGFBP-7, IL-6, CCL-21, protein S, angiogenin or any combination thereof. In some embodiments, an FPSec for liver fibrosis risk assessment may comprise a combination of four or more circulating proteins of MMP-7, VCAM-1, IGFBP-7, IL-6, CCL-21, protein S, angiogenin or any combination thereof. In some embodiments, an FPSec for liver fibrosis risk assessment may comprise a combination of five or more circulating proteins of MMP-7, VCAM-1, IGFBP-7, IL-6, CCL-21, protein S, angiogenin or any combination thereof. In some embodiments, an FPSec for liver fibrosis risk assessment may comprise a combination of six or more circulating proteins of MMP-7, VCAM-1, IGFBP-7, IL-6, CCL-21, protein S, angiogenin or any combination thereof. In some embodiments, an FPSec for liver fibrosis risk assessment may comprise a combination of the circulating proteins of MMP-7, VCAM-1, IGFBP-7, IL-6, CCL-21, protein S, angiogenin or any combination thereof,

In some embodiments, an FPSec for liver fibrosis risk assessment may comprise a combination of one or more circulating proteins wherein at least one of the proteins is a high-risk-associated protein. In some embodiments, an FPSec for liver fibrosis risk assessment may comprise a combination of one or more high-risk-associated circulating proteins of vascular cell adhesion molecule 1 (VCAM-1), insulin-like growth factor-binding protein 7 (IGFBP-7), matrix metallopeptidase 7 (MMP-7), interleukin-6 (IL-6), and C-C motif chemokine ligand 21 (CCL-21) or any combination thereof. In some embodiments, an FPSec for liver fibrosis risk assessment may comprise a combination of one or more circulating proteins wherein at least one of the proteins is a low-risk-associated protein. In some embodiments, an FPSec for liver fibrosis risk assessment may comprise a combination of one or more low-risk-associated circulating proteins of protein S, angiogenin or any combination thereof. In some embodiments, an FPSec for liver fibrosis risk assessment may comprise a combination of one or more high-risk-associated circulating proteins and one or more low-risk-associated circulating proteins. In some embodiments, an FPSec for liver fibrosis risk assessment may comprise a combination of one or more high-risk-associated circulating proteins of VCAM-1, IGFBP-7, MMP-7, IL-6, CCL-21, or any combination thereof and one or more low-risk-associated circulating proteins of protein S, angiogenin or any combination thereof.

### (c) FPS and FPSec Assays and FPS and FPSec Scores

In some embodiments, an FPS as disclosed herein may be used to determine an FPS score. In some embodiments, an FPSec as disclosed herein may be used to determine an FPSec score. In some embodiments, an FPS score and/or an FPSec score can be determined from one or more samples collected from a subject as described herein. In some embodiments, an FPSec score can be determined from the results of an FPSec assay. In some embodiments, an FPS score can be determined from the results of an FPS assay.

### FPS Assay and FPS Score

In some embodiments, a sample collected from a subject as disclosed herein can be processed and used in an FPS assay. As used herein, an “FPS assay” refers to subjecting a sample to any method suitable for determining the level of gene expression of any one of the genes comprising an FPS for liver fibrosis risk assessment as disclosed herein. In some embodiments, an FPS assay may be a method of measuring gene expression of one or more genes within an FPS for liver fibrosis risk assessment. In some embodiments, an FPS assay may be a method of measuring gene expression of one or more of AEBP1, ANXA1, ASAHL (NAAA), BCL2, CCL21, CXCR4, DDR1, F9, FBN1, FILIP1L, HAAO, IER3, IGFBP6, KRT7, LOXL2, NTS, PMM1, PON3, SLC7A1, TTR, or any combination thereof within an FPS for liver fibrosis risk assessment. In some embodiments, an FPS assay may be a method of measuring gene expression of one or more of AEBP1, ANXA1, IER3, CXCR4, FILIP1L, LOXL2, KRT7, DDR1, SLC7A1, BCL2, NTS, FBN1, IGFBP6, ASAHL/NAAA, TTR, PMM1, PON3, F9, HAAO, or any combination thereof within an FPSec for liver fibrosis risk assessment. In some embodiments, an FPS assay may be a method of measuring gene expression of one or more of ANXA1, AEBP1, FBN1, IER3, CCL21, CXCR4, KRT7, IGFBP6, FILIP1L LOXL2, BCL2, SLC71, DDR1, and NTS or any combination thereof within an FPS for liver fibrosis risk assessment. In some embodiments, an FPSec assay may be a method of measuring gene expression of one or more of PMM1, NAAA, TTR, PON3, HAAO, F9 or any combination thereof within an FPS for liver fibrosis risk assessment. In some embodiments, an FPS assay may be a method of measuring gene expression of (a) one or more of ANXA1, AEBP1, FBN1, IER3, CCL21, CXCR4, KRT7, IGFBP6, FILIP1L LOXL2, BCL2, SLC71, DDR1, and NTS or any combination thereof and (b) one or more of PMM1, NAAA, TTR, PON3, HAAO, F9 or any combination thereof within an FPS for liver fibrosis risk assessment.

In some embodiments, an FPS assay described herein may entail subjecting a sample from a subject herein to a gene expression profiling assay of one or more genes within an FPS for liver fibrosis risk assessment. A gene expression profiling assay is a type of assay that uses labeled oligonucleotide probes to simultaneously measure multiple RNA transcripts in a sample in a single experiment. Non-limiting examples of gene expression profiling assays suitable for use herein may include digital transcript counting assays like the nCounter Analysis System (NanoString). Alternatively, the gene expression profiling assay may include a DNA microarray or RNA-Seq assay. In some embodiments, an FPS assay may entail subjecting a sample collected from a subject herein to an FDA-approved clinical diagnostic digital transcript counting technology, nCounter platform.

In some embodiments, an FPS assay may entail subjecting a sample from a subject herein to a multi-analyte profiling assay for gene expression of one or more genes (e.g., 1, 2, 3, 4, 5, 6, 7, 8, 9, 10, 11, 12, 13, or 14) within a gene panel of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9 or any combination thereof. In some embodiments, an FPS assay may entail subjecting a sample from a subject herein to a multi-analyte profiling assay for gene expression of one or more genes (e.g., 1, 2, 3, 4, 5, 6, 7, 8, 9, 10, 11, 12, 13, or 14) within a gene panel of ANXA1, AEBP1, FBN1, IER3, CCL21, CXCR4, KRT7, IGFBP6, FILIP1L LOXL2, BCL2, SLC71, DDR1, NTS, or any combination thereof. In some embodiments, an FPS assay may entail subjecting a sample from a subject herein to a multi-analyte profiling assay for gene expression of one or more genes (e.g., 1, 2, 3, 4, 5, 6, 7) within a gene panel of PMM1, NAAA, TTR, PON3, HAAO, F9 or any combination thereof. In some embodiments, an FPS assay may entail subjecting a sample from a subject herein to a multi-analyte profiling assay for gene expression of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9.

In some embodiments, an FPS assay may entail subjecting a sample from a subject herein to a multi-analyte profiling assay for gene expression of one or more genes (e.g., 1, 2, 3, 4, 5, 6, 7, 8, 9, 10, 11, 12, 13, or 14) within a gene panel of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9 or any combination thereof and normalizing the gene expression measurements. One of skill in the art will appreciate that the method of normalizing gene expression measurements will depend upon the specifics of the multi-analyte profiling assay used. In accordance with some of the embodiments herein, an FPS assay may entail subjecting a sample from a subject herein to a multi-analyte profiling assay for gene expression of one or more genes (e.g., 1, 2, 3, 4, 5, 6, 7, 8, 9, 10, 11, 12, 13, or 14) within a gene panel of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9 or any combination thereof and normalizing the gene expression measurements to gene expression levels of a set of control genes (e.g., BAT3, NDUFA2, COX8A, HNRNPA2B1, HINT1, ATP5B). In various aspects, normalizing the gene expression measurements comprises quantifying raw expression of each gene as a digital count of its transcript and then dividing each digital count by the geometric mean of the raw counts of the control genes.

In some embodiments, normalized gene expression measurements produced by an FPS assay herein may be used to generate an FPS score. In general, the method of determining an FPS score generally involves comparing a gene expression profile of a subject to a gene expression profile of a reference profile (or template) of high risk and a reference profile (or template) of low risk for liver fibrosis progression. In some embodiments this involves converting normalized gene expression measurements produced by an FPS assay herein into a predicted confidence p value with proximity to either a high-risk reference profile or a low-risk reference profile, and then using the predicted confidence p value with proximity to either of the high or low-risk reference profile to calculate the FPS score, as described below. In some embodiments, normalized gene expression measurements produced by an FPS assay herein may be converted into high or low risk genes by top quartile cut-off in the optimization set, wherein a high-risk gene is ANXA1, AEBP1, FBN1, IER3, CCL21, CXCR4, KRT7, IGFBP6, FILIP1L LOXL2, BCL2, SLC71, DDR1, and/or NTS and a low-risk gene is PMM1, NAAA, TTR, PON3, HAA and/or F9. Accordingly, a high risk reference profile may be generated as a vector concatenating 1 for the 14 high-risk-associated genes and 0 for the 6 low-risk-associated genes, i.e., (1, 1, 1, 1, 1, 1, 1, 1, 1, 1, 1, 1, 1, 1, 0, 0, 0, 0, 0, 0) and the low-risk reference profile may be generated as a vector concatenating 0 for the 14 high-risk-associated genes and 1 for the 6 low-risk-associated genes, i.e., (0, 0, 0, 0, 0, 0, 0, 0, 0, 0, 0, 0, 0, 0, 1, 1, 1, 1, 1, 1). These high or low risk reference profiles are then used in the methods below to derive the FPS score for a sample.

In various aspects, converting normalized gene expression measurements (herein referred to as a “gene expression profile”) into an FPS score comprises the following steps. In a first step, the normalized gene expression measurements are compared to the high and low risk reference profiles described above. In some aspects, comparing the gene expression profile to the high and low risk reference profiles comprises quantifying the similarity between the gene expression profile and each of the high or low risk reference profile. The similarity of the gene expression profile to either of the reference profiles may in some aspects be quantified by cosine distance. In various aspects, the risk reference profile (e.g., the high or low risk reference profile) having the lowest cosine distance (highest similarity to the gene expression profile) is then selected and a prediction confidence p value for the gene expression profile in reference to (i.e., “with proximity to”) that risk reference profile is calculated based on random permutation test. This prediction confidence p value is assigned a sign depending on which risk reference profile is used in its derivation (e.g., positive for high-risk reference profile and negative for low-risk reference profile) and converted to a logarithmic scale (base 10) with negative sign to generate the FPS score. For example, a prediction confidence p value with proximity to the high-risk reference profile of 0.05 would correspond to an FPS score of +1.3013 (+(−log ₁₀ (0.05))=+1.3013)). A prediction confidence p value with proximity to the low risk reference profile of 0.05 would correspond to an FPS score of −1.3013 (−(−log ₁₀ (0.05))=−1.3013). When the p value is less than 0.05 with proximity to the high-risk reference profile (FPS score greater than +1.3013), the subject is at high risk (has a “similar” gene expression profile to the theoretical patients with the highest risk of fibrosis progression). When the p value is less than 0.05 with proximity to the low-risk reference profile (FPS score less than −1.3013), the subject is at low risk (has a “similar” gene expression profile to the theoretical patients with the lowest risk of fibrosis progression). When the p value is equal or greater than 0.05 with proximity to either reference profile (FPS score between −1.3013 and +1.3013), the subject is at intermediate risk. In each case, the FPS score is a measure of how similar the gene expression profile of the subject is to either of the reference profiles (wherein the high- and low-risk reference profiles represent the theoretical upper and lower limit for the range of risk level, respectively).

Therefore, in various aspects, the FPS scores herein are provided as a measure of a risk of an individual for developing long-term liver fibrosis progression. In some embodiments, a subject having an FPS score as determined herein above a given threshold (e.g., +1.30103, corresponding to a prediction confidence p-value of 0.05 with proximity to the high-risk reference profile) may be predicted to be at high risk for developing liver fibrosis or long-term liver fibrosis progression. In some embodiments, a subject having an FPS score as determined herein below a given threshold (e.g., −1.30103, corresponding to a prediction confidence p-value of 0.05 with the low-risk reference profile) may be predicted to be at low risk for developing liver fibrosis and/or long-term liver fibrosis progression. In some embodiments, a subject having an FPS score as determined herein between these two thresholds (e.g., −1.30103 to +1.30103, which correspond to a prediction confidence p-value of 0.05 irrespective of closer reference profile) may be predicted to be at intermediate risk for developing liver fibrosis and/or long-term liver fibrosis progression.

### FPSec Assay and FPSec Score

In some embodiments, a sample collected from a subject as disclosed herein can be processed and used in an FPSec assay. As used herein, a “FPSec assay” refers to subjecting a sample to any method suitable for determining the level of protein expression of any one of the proteins comprising an FPSec for liver fibrosis risk assessment as disclosed herein. In some embodiments, an FPSec assay may be a method of measuring protein abundance of one or more proteins within an FPSec for liver fibrosis risk assessment. In some embodiments, an FPSec assay may be a method of measuring protein abundance of one or more of: vascular cell adhesion molecule 1 (VCAM-1), insulin-like growth factor-binding protein 7 (IGFBP-7), matrix metallopeptidase 7 (MMP-7), interleukin-6 (IL-6), C-C motif chemokine ligand 21 (CCL-21), angiogenin, protein S or any combination thereof within an FPSec for liver fibrosis risk assessment. In some embodiments, an FPSec assay may be a method of measuring protein abundance of one or more of VCAM-1, IGFBP-7, MMP-7, IL-6, CCL-21, protein S, angiogenin or any combination thereof within an FPSec for liver fibrosis risk assessment. In some embodiments, an FPSec assay may be a method of measuring protein abundance of two to six (e.g., 2, 3, 4, 5, 6) or more of VCAM-1, IGFBP-7, MMP-7, IL-6, CCL-21, protein S, angiogenin, or any combination thereof within an FPSec for liver fibrosis assessment. In some embodiments, an FPSec assay may be a method of measuring protein abundance of an FPSec for liver fibrosis risk assessment, wherein the FPSec may be a protein panel of VCAM-1, IGFBP-7, MMP-7, IL-6, CCL-21, protein S, and angiogenin.

In some embodiments, an FPSec assay described herein may entail subjecting a sample from a subject herein to a multi-analyte profiling assay for protein quantification of one or more proteins within an FPSec. A multi-analyte profiling assay (xMAP; also known as a multiplex assay) is a type of immunoassay that uses magnetic beads to simultaneously measure multiple analytes in a single experiment. A multiplex assay is a derivative of an ELISA using beads for binding the capture antibody. Non-limiting examples of multi-analyte profiling (xMAP) assays suitable for use herein may include Myriad RBM MAP Luminex xMAP, and/or bead array assays performed on either multi-use flow cytometers (such as the commonly available clinical cytometers from Becton Dickinson, Beckman-Coulter, Dako-Cytomation, or Partec). In some embodiments, an FPSec assay may entail subjecting a sample collected from a subject herein to a FDA-approved multiplex clinical diagnostic technology, xMAP platform (e.g., Luminex).

In some embodiments, an FPSec assay may entail subjecting a sample from a subject herein to a multi-analyte profiling assay for protein quantification of one or more proteins (e.g., 1, 2, 3, 4, 5, 6, 7) within a protein panel of VCAM-1, IGFBP-7, MMP-7, IL-6, CCL-21, protein S, angiogenin or any combination thereof. In some embodiments, an FPSec assay may entail subjecting a sample from a subject herein to a multi-analyte profiling assay for protein quantification of VCAM-1, IGFBP-7, MMP-7, IL-6, CCL-21, protein S, and angiogenin.

In some embodiments, an FPSec assay may entail subjecting a sample from a subject herein to a multi-analyte profiling assay for protein quantification of one or more proteins (e.g., 1, 2, 3, 4, 5, 6, 7) within a protein panel of VCAM-1, IGFBP-7, MMP-7, IL-6, CCL-21, protein S, angiogenin or any combination thereof and normalizing the protein quantification measurements. One of skill in the art will appreciate that the method of normalizing the protein quantification measurements will depend upon the specifics of the multi-analyte profiling assay used. In accordance with some of the embodiments herein, an FPSec assay may entail subjecting a sample from a subject herein to a multi-analyte profiling assay for protein quantification of one or more proteins (e.g., 1, 2, 3, 4, 5, 6, 7) within a protein panel of VCAM-1, IGFBP-7, MMP-7, IL-6, CCL-21, protein S, angiogenin, or any combination thereof and normalizing the protein quantification measurements to median fluorescent intensity.

In some embodiments, normalized protein quantification measurements produced by an FPSec assay herein may be used to generate an FPSec score. In some embodiments, normalized protein quantification measurements produced by an FPSec assay herein may be converting into an aggregated score, wherein the aggregated score is the FPSec score. In some embodiments, normalized protein quantification measurements produced by an FPSec assay herein may be converted into high or low abundance by top quartile cut-off in the optimization set, and calculated a semiquantitative score according to Formula I:

$$2 + {\sum }_{i = 1}^{8} ( \begin{matrix}1 for high abundance of high risk protein \\ - 1 for high abundance of low risk protein \\ 0 otherwise\end{matrix} ) for probe i , \qquad ( I )$$

wherein a high-risk protein is VCAM-1, IGFBP-7, MMP-7, IL-6, and/or CCL-21, and a low risk protein is protein S and/or angiogenin.

In some embodiments, a subject having an FPSec score as determined herein below 3 may be predicted to be at low risk for developing liver fibrosis and/or long-term liver fibrosis progression. In some embodiments, a subject having an FPSec score as determined herein of 3 or higher may be predicted to be at high risk for developing liver fibrosis and/or long-term liver fibrosis progression.

In some embodiments, methods of determining an FPS score and/or an FPSec score as disclosed herein may identify a subject in need of risk-based liver fibrosis progression screening. For subjects deemed to be at risk for developing a liver fibrosis and/or recovering from a liver condition that is associated with liver fibrosis, current practice guidelines recommend regular liver fibrosis screening. Non-limited examples of liver fibrosis screening methods can include measuring circulating cell-free methylated DNA, ultrasound, magnetic resonance imaging (MRI), computed tomography (CT), and the like. In some embodiments, methods of determining an FPSec score as disclosed herein may identify a subject in need of risk-based liver fibrosis screening to be performed at least once a year. In some aspects, methods of determining an FPSec score as disclosed herein may identify a subject in need of risk-based liver fibrosis screening to be performed about once a year to about six-times a year (e.g., about once, twice, three-times, four-times, five-times, six-times a year). In some examples, methods of determining an FPSec score as disclosed herein may identify a subject in need of risk-based liver fibrosis screening to be performed about twice a year.

In some embodiments, methods of diagnosing a liver fibrosis or liver fibrosis progression in a subject may entail performing an FPS and/or FPSec assay and/or determining an FPS and/or FPSec score as disclosed herein. In some embodiments, methods of diagnosing liver fibrosis in a subject having or suspected of having liver fibrosis may entail performing an FPS and/or an FPSec assay and/or determining an FPS and/or an FPSec score as disclosed herein in addition to performing a liver biopsy, one or more blood tests to assess liver function, computed tomography, magnetic resonance imaging, or any combination thereof. In some aspects, the one or more blood tests performed to assess liver function may be a measurement of alanine transaminase (ALT), aspartate transaminase (AST), alkaline phosphatase (ALP), albumin, bilirubin, gamma-glutamyltransferase (GGT), L-lactate dehydrogenase (LD), prothrombin time (PT), or any combination thereof.

### III. Methods of Treating Liver Fibrosis in a Subject

In general, methods disclosed herein include treating a subject having or suspected of having a liver fibrosis progression by performing an FPSec assay to measure protein abundance of one or more of the circulating proteins associated with FPSec as disclosed herein, obtaining an FPSec score from the FPSec assay results, and administering the appropriate treatment based on the FPSec score. In some embodiments, treatment after determining the FPSec score as disclosed herein may depend on if the FPSec score is indicative of a high risk for liver fibrosis progression (e.g., greater than or equal to 3) or a low risk for liver fibrosis progression (e.g., less than 3).

Further methods disclosed herein include treating a subject having or suspected of having a liver fibrosis progression by performing an FPS assay to measure gene expression of one or more of the genes associated with FPS as disclosed herein, obtaining an FPS score from the FPS assay results, and administering the appropriate treatment based on the FPS score. In some embodiments, treatment after determining the FPS score as disclosed herein may depend on if the FPS score is indicative of a high risk for liver fibrosis progression (e.g., greater than +1.30103, corresponding to a prediction confidence p value of 0.05 with proximity to the high-risk reference profile) or a low risk for liver fibrosis progression (e.g., less than −1.30103, corresponding to a prediction confidence p value of 0.05 with proximity to the low-risk reference profile) or an intermediate risk for liver fibrosis progression (e.g., between −1.30103 and +1.30103, corresponding to a prediction confidence p value of 0.05 with proximity to either of the high-risk reference profile or low-risk reference profile).

A suitable tailored treatment approach for liver fibrosis progression as used herein may be selected based on the subject's diagnosis and/or classification of the liver condition or disease associated with or causing the liver fibrosis. In some embodiments, a subject can be diagnosed with liver fibrosis progression based on increased protein abundance of one or more circulating protein markers that make up a serum-protein-based FPSec as disclosed herein. In some embodiments, a subject can be diagnosed with liver fibrosis progression based on increased gene expression of one or more genes that make up an FPS as disclosed herein. In some embodiments, a subject can be predicted to have a high or low risk for liver fibrosis progression based on increased protein abundance of one or more circulating protein markers that make up a serum-protein-based FPSec as disclosed herein. In some embodiments, a subject can be predicted to have a high or low risk for liver fibrosis progression based on increased gene expression of one or more genes that make up an FPS as disclosed herein. In some embodiments, a subject can be classified as having a high or low risk for liver fibrosis progression based on increased protein abundance of one or more circulating protein markers that make up a serum-protein-based FPSec as disclosed herein. In some embodiments, a subject can be classified as having a high or low risk for liver fibrosis progression based on increased gene expression of one or more genes that make up an FPS disclosed herein

In some embodiments, a subject can be diagnosed and/or predicted to have high or low risk for liver fibrosis progression based on methods of determining an FPS and/or FPSec score as disclosed herein in addition to an assessment of at least one disease, condition, or combination thereof that predisposes the subject to liver fibrosis. In some embodiments, further assessment of at least one disease, condition, or combination thereof that predisposes the subject to liver cancer (e.g., HCC) may include at diagnosis and/or a determination of severity of chronic infection of hepatitis B virus (HBV), chronic infection of hepatitis C virus (HCV), non-alcoholic fatty liver disease (NAFLD), non-alcoholic steatohepatitis (NASH), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof. Methods of diagnosing these diseases and conditions are known in the art. (See e.g., HARRISON'S PRINCIPLES OF INTERNAL MEDICINE, 18e. New York, NY: McGraw-Hill; 2012.)

In some embodiments, a subject can be diagnosed and/or predicted to have high or low risk for liver fibrosis progression based on methods of determining an FPS and/or FPSec score as disclosed herein and be further diagnosed with liver fibrosis via an additional method. In some embodiments, an additional method of diagnosing liver fibrosis that can be used in addition to determination of an FPS and/or FPSec score may be histological or imaging-based (contrast-enhanced multiphase CT, ultrasound, and/or MRI) examinations according to the American Association of the Study of Liver Disease (AASLD) practice guidelines. Imaging features used to diagnose a liver fibrosis include size, kinetics, and pattern of contrast enhancement, and growth on serial imaging wherein size may be measured as the maximum cross-section diameter on the image where the scarring is most clearly seen.

In some embodiments, an FPS and/or FPSec score may be obtained using the methods herein to determine one or more treatment options for liver fibrosis progression in a subject. In some embodiments, an FPS and/or FPSec score may be obtained using the methods herein to determine one or more treatment options for liver fibrosis progression in a subject in conjunction with one or more additional factors. In some aspects, treatment options for liver fibrosis progression in a subject herein may depend on an FPS and/or an FPSec score as disclosed herein and one or more of the following additional factors: presence or absence of cirrhosis; operative risk based on extent of cirrhosis and comorbid diseases; overall performance status; portal vein patency; or any combination thereof.

In some embodiments, an FPS and/or FPSec score may be obtained using the methods herein to determine one or more treatment options for liver fibrosis progression in a subject wherein the one or more treatments may include surgical removal of one or more fibrotic scars, liver transplant, drug therapy, or any combination thereof. In some aspects, the treatment comprises an anti-fibrotic therapy. In some aspects, the anti-fibrotic therapy may include administration of one or more drugs to the subject, wherein the drugs are comprised of galunisertib, erlotinib, AM095, bortezomib, pioglitazone, metformin, epigallocatechin gallate (EGCG), I-BET 151, JQ1, captopril, and nizatidine (Selleck Chemicals); MG-132; and cenicriviroc. One of skill in the art will appreciate that dosing regimens can vary and require optimization for a subject to be treated based on the various factors such as that subject's age, weight, gender, renal/liver function, and the like. In some embodiments, any of the methods disclosed herein can further include monitoring occurrence of one or more adverse effects in the subject having an FPS and/or FPSec score indicative of a high-risk for liver fibrosis. Adverse effects may include, but are not limited to, hepatic impairment, hematologic toxicity, neurologic toxicity, cutaneous toxicity, gastrointestinal toxicity, or a combination thereof. When one or more adverse effects are observed, the methods disclosed herein can further include reducing or increasing the dose of one or more of the treatment regimens depending on the adverse effect or effects in the subject. For example, when a moderate to severe hepatic impairment is observed in a subject after treatment, compositions of use to treat the subject can be reduced in concentration or frequency of dosing with one or more disclosed drugs.

In certain embodiments, the FPS and/or FPSec score may be monitored in an individual before and after treatment. In some cases, changes in the FPS and/or FPSec score may lead to continuing or discontinuing the treatment. For example, if the FPS and/or FPSec score in a subject decreases after treatment, the treatment may be continued. If the FPS and/or FPSec score in a subject increases or doesn't change after treatment, the treatment may be discontinued.

In some aspects, treatment of a subject after determining the FPS and/or FPSec score as disclosed herein, may prevent liver fibrosis progression. In some aspects, treatment of a subject after determining the FPS and/or FPSec score as disclosed herein, may ameliorate one or more symptoms associated with liver fibrosis. In still other aspects, treatment of a subject after determining the FPS and/or FPSec score as disclosed herein, may reduce risk of liver fibrosis recurrence in the subject. In other aspects, treatment of a subject after determining the FPS and/or FPSec score as disclosed herein, may slow fibrosis progression in the liver of the subject. In some other aspects, treatment of a subject after determining the FPS and/or FPSec score as disclosed herein, may reduce the risk of cirrhosis in the subject.

In some embodiments, methods of treatment disclosed herein can impair liver fibrosis progression compared to liver fibrosis progression in an untreated subject with identical disease condition and predicted outcome. In some embodiments, liver fibrosis progression can be stopped following treatments according to the methods disclosed herein. In other embodiments, liver fibrosis progression can be impaired at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other words, liver tumors in subject treated according to the methods disclosed herein grow at least 5% less (or more as described above) when compared to an untreated subject with identical disease condition and predicted outcome. In some embodiments, liver fibrosis progression can be impaired at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, liver fibrosis progression can be impaired at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

In some embodiments, treatment of liver fibroses according to the methods disclosed herein can result in a shrinking of a liver fibrosis in comparison to the starting size of the liver fibrosis. In some embodiments, liver fibrosis shrinking may be at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the liver fibrosis is completely gone after treatment) compared to the starting size of the liver fibrosis.

In various embodiments, treatments administered according to the methods disclosed herein can improve patient life expectancy compared to the life expectancy of an untreated subject with identical disease condition (e.g., NAFLD) and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

### V. Kits

The present disclosure provides kits for performing any of the methods disclosed herein. In some aspects, the present disclosure provides a kit for determining expression of one or more markers of liver fibrosis as disclosed herein and for diagnosing the liver fibrosis. Such a kit may comprise a means for determining any of the combinations proteins that make up a panel of circulating proteins referred to as a serum-protein-based FPSec as disclosed herein. Alternatively, such kits may comprise a means for determining any of the combination genes that make up a panel of genes referred to as FPS as disclosed herein.

In some embodiments, the means for determining expression of one or more circulating proteins of FPSec as disclosed herein may have a set of antibodies, peptides, aptamers, or any combination thereof. In some examples, a means for determining expression of one or more circulating proteins of FPSec disclosed herein may have a set of antibodies/antigens. Each of the antibodies/antigens can detect a target circulating protein of FPSec in the combination and the whole set, collectively, may be designed for detecting at least two, at least three, at least four, at least five, at least six, or at least seven FPSec proteins (e.g., VCAM-1, IGFBP-7, MMP-7, IL-6, CCL-21, angiogenin, protein S) in combination. Design of such antibodies/antigens for detecting a particular protein using xMAP assay is within the knowledge of a skilled person in the art. See, e.g., Sambrook et al. et al., MOLECULAR CLONING-A LABORATORY MANUAL (2ND ED.), Vols. 1-3, Cold Spring Harbor Laboratory, Cold Spring Harbor, N.Y. (1989).

Likewise, in some embodiments, the means for determining expression of one or more genes of FPS as disclosed herein may have a set of nucleic acid probes, primers, oligonucleotides or any other means to detect levels of a nucleic acid. For example, in some aspects the means for determining the gene expression profile of one or more genes of FPS disclosed herein may be a set of nucleic acid probes labeled with color-coded microbeads to mRNA transcribed from one or more genes in the FPS assay (e.g., ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9). In some examples, a means for determining the gene expression profile of one or more genes of FPS disclosed herein may be a set of primers and/or oligonucleotides. Each oligonucleotide may detect a target gene or an mRNA transcribed from a target gene of FPS in the combination and the whole set, collectively, may be designed for detecting at least two, at least three, at least four, at least five, at least six, or at least seven, at least eight, at least nine, at least ten, at least eleven, at least twelve, at least thirteen, at least fourteen, at least fifteen, at least sixteen, at least seventeen, at least eighteen, at least nineteen or at least twenty FPS genes (e.g., ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9) in combination. Design of such oligonucleotides, primers or probes for detecting expression of particular genes using microarrays is within the knowledge of a skilled person in the art. See, e.g., Sambrook et al. et al., MOLECULAR CLONING-A LABORATORY MANUAL (2ND ED.), Vols. 1-3, Cold Spring Harbor Laboratory, Cold Spring Harbor, N.Y. (1989). In exemplary embodiments, the means for determining expression of one or more genes of FPS herein may include standard components included in an nCounter® assay (NanoString Inc).

In some embodiments, kits disclosed herein can have a solid support member, on which the set of antibodies or nucleic acids (e.g., “probes”) can be immobilized. In some examples, kits disclosed herein may comprise a platform comprising a support member, on which the set of probes can be immobilized. The probes may have oligonucleotide or peptide molecules that bind to a specific target molecule. The support member in the platform may be either porous or non-porous. For example, the probes may be attached to a nitrocellulose or nylon membrane or to a bead. Alternatively, the support member may have a glass or plastic surface. In some examples, the solid phase may be a nonporous or, optionally, a porous material such as a gel.

In some embodiments, a platform array may comprise a support member with an ordered array of binding (e.g., hybridization) sites or “probes” each representing one of the target protein or gene markers described herein. Preferably the platform arrays are addressable arrays, and more preferably positionally addressable arrays. For example, each probe of the array is preferably located at a known, predetermined position on the solid support such that the identity (i.e., the sequence) of each probe can be determined from its position in the array (i.e., on the support or surface). In preferred embodiments, each probe is covalently attached to a solid support. In some aspects, the solid support may be a bead.

Any of the kits disclosed herein may further comprise a container for placing a biological sample, and optionally a tool for collecting a biological sample from a subject. Alternatively, or in addition, the kit may further comprise one or more reagents for determining protein levels of the one or more circulating proteins of FPSec as disclosed herein from the biological sample. In some examples, the kit may comprise reagents for immunodetection of one or more circulating proteins of FPSec as disclosed herein. Alternatively, or in addition, the kit may further comprise one or more reagents for determining gene expression levels of the one or more genes of FPS as disclosed herein from the biological sample. In some examples, the kit may comprise reagents for detecting gene expression of one or more genes in FPS as disclosed herein using a microarray. In other examples, the kit may comprise reagents for hybridization.

Any of the kits may further comprise an instruction manual providing guidance for using the kit to determine a protein panel and/or gene expression profile having any combination of the one or more circulating proteins of FPSec and/or one or more genes of FPS as disclosed herein.

Further, any of the kits disclosed herein may comprise a processor, e.g., a computational processor, for assessing abundance of one or more of the circulating proteins of FPSec and/or one or more expressed genes of FPS as disclosed herein. Such a processor may be configured with a regression model such as those disclosed herein. By inputting the marker profile (e.g., the protein expression level of circulating proteins of FPSec or the gene expression profile of genes of FPS), the processor may process the information to diagnose liver fibrosis and optionally diagnose the level of liver fibrosis severity by generating an FPSec score and/or an FPS score according to the methods disclosed herein.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### Examples

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Materials And Methods

### Patients and Specimens

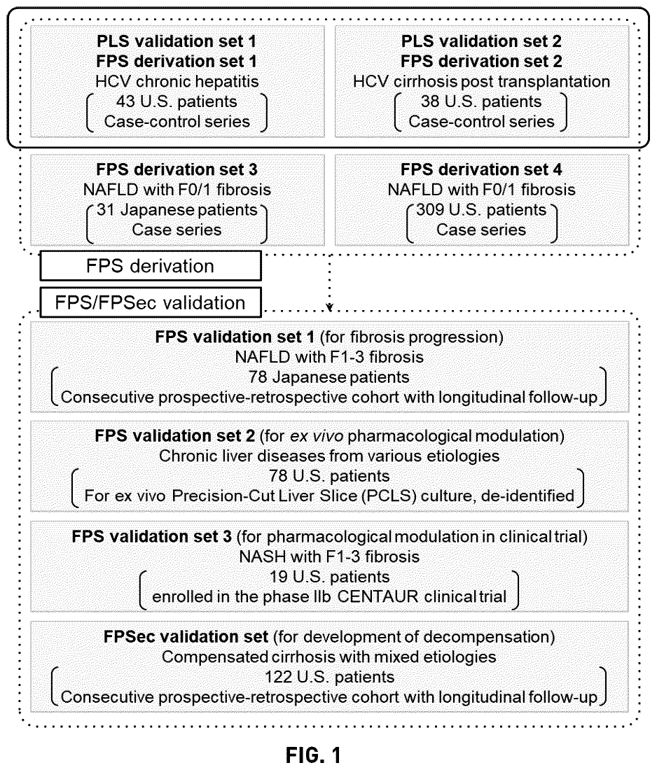

Archived formalin-fixed liver tissues from index biopsy were used for histological assessment in all patients (FIG. 1 and Table 1A-11) to confirm no to minimal fibrosis (METAVIR fibrosis stage F0 or F1). The PLS validation set 1 (and FPS derivation set 1) is a case-control series of 43 chronic hepatitis C patients from a prior cohort study consecutively diagnosed and followed at Johns Hopkins and Massachusetts General Hospital between 1998 and 2010. Twenty-five patients were co-infected with HIV and on anti-retroviral therapies. The patients were regularly followed up with ultrasound elastography at median interval of 1.1 (IQR: 0.6-2.0) years. Liver stiffness measurement >7.0 and >9.5 kPa were regarded as indication of F2 and F3 fibrosis, respectively. The PLS validation set 2 (and FPS derivation set 2) is a case-control series of 38 patients who consecutively underwent liver transplantation for HCV-related cirrhosis and protocol liver biopsies at year 1, 2, and 5 after transplantation (and additional biopsies as needed to evaluate graft rejection, which were excluded) at Baylor University between 2002 and 2007. Median number of biopsies was 8 (IQR: 6-9) per patient with median interval between serial biopsies of 8.6 (IQR: 0.3-12.6) months. The cases showed F-stage increase of two stages or more within 5 years of follow-up, and the controls were defined as patients who were free from the fibrosis progression for 5 years or more and matched for sex, age (at 5-year interval), and F-stage at baseline. The FPS derivation set 3 is a cross-sectional series of 31 NAFLD patients who underwent diagnostic liver biopsy at Hiroshima University between 2003 and 2015. The FPS derivation set 4 is a cross-sectional series of 309 NAFLD patients who underwent diagnostic liver biopsy (F0 or F1 fibrosis) at Massachusetts General Hospital between 2009 and 2016. The FPS validation set 1 for this study's primary endpoint, fibrosis progression of one stage or more, is comprised on a case-control series of 78 NASH patients with F1 to F3 fibrosis in index liver biopsy who had a follow-up biopsy to investigate histological disease progression at median interval of 2.4 (IRQ: 2.2-3.0) years at Hiroshima University between 2004 and 2018. The cases were defined as patients who had F-stage increase of one stage or more in the follow-up biopsy. The FPS validation set 2 includes 78 patients with fibrotic liver diseases from various etiologies, for which de-identified fresh liver tissues were available from standard-care hepatic resection for organotypic ex vivo tissue culture at University of Texas Southwestern and Mount Sinai. The FPS validation set 3 consists of NASH patients with F1-F3 fibrosis who underwent liver biopsy before and after 1-year treatment with cenicriviroc (n=9) or placebo (n=10) in the phase Ilb CENTAUR trial (NCT02217475). Decrease of one or more fibrosis stage was regarded as anti-fibrotic response. The serum surrogate FPS was assessed in archived de-identified serum samples from 79 patients with chronic liver diseases. The FPSec validation set is a cohort of 122 patients with compensated (Child-Pugh class A) cirrhosis patients with mixed etiologies enrolled at University of Michigan between 2004 and 2006 as reported in our previous study. Hepatic decompensation was defined as newly developed massive ascites, hepatic encephalopathy, bleeding from gastroesophageal varices, or liver transplantation. The study was approved by institutional review board at respective institutions with written informed consent or exemption for use of archived de-identified samples (protocol numbers: STU062018-058, STU072018-071, 2010P000220/PHS, HS13-00159).

TABLE 1

| | | | | |
|---|---|---|---|---|
| Variable | All cases | Cases | Controls | P |

TABLE 1A : PLS validation set 1, FPS derivation set 1 (chronic hepatitis
C, case-control, U.S.), n = 43 (case:control = 12:31)

| | | | | |
|---|---|---|---|---|
| Age, y | 46 (42-49) | 48 (45-49) | 45 (41-50) | .21 |
| Male sex, n (%) | 35 (81%) | 11 (92%) | 24 (77%) | .41 |
| Total bilirubin, | 0.6 (0.4-1.0) | 0.6 (0.4-0.9) | 0.6 (0.4-1.0) | .80 |
| mg/dL | | | | |
| Albumin, g/dL | 4.0 (3.8-4.3) | 4.0 (3.7-4.2) | 4 (3.8-4.3) | .67 |
| AST, IU/L | 41 (30.2- | 69 (50.0- | 35 (29.5- | .004 |
| | 66.8) | 75.5) | 45.0) | |
| ALT, IU/L | 42 (31.5- | 59.5 (46.0- | 36 (30.0- | .011 |
| | 64.5) | 92.5) | 54.0) | |
| Platelet count, | 218 (185.0- | 193.5 (177.8- | 228 (192.5- | .044 |
| ×10³/mL | 249.0) | 219.2) | 255.0) | |
| HIV co-infection, | 24 (56%) | 9 (75%) | 15 (48%) | .17 |
| n (%) | | | | |

TABLE 1B: PLS validation set 2, FPS derivation set 2 (transplantation,
case-contorl = 21:17)

| | | | | |
|---|---|---|---|---|
| Donor/recipient | 42 (23-47)/52 | 28 (23- | 42 (37-46)/47 | .27/ |
| age, y | (46-55) | 50)/54 | (44-52) | .02 |
| | | (50-53) | | |
| Male sex, n (%) | 25 (66%) | 11 (52%) | 14 (82%) | .09 |
| Total bilirubin, | 0.7 (0.4-0.9) | 0.8 (0.5-0.9) | 0.5 (0.5-0.8) | .09 |
| mg/dL | | | | |
| Albumin, g/dL | 4.0 (3.7-4.1) | 3.9 (3.7-4.1) | 4.0 (3.9-4.2) | .26 |
| AST, IU/L | 55 (37-112) | 71 (46-177) | 39 (33-58) | .02 |
| ALT, IU/L | 60 (46-115) | 78 (48-206) | 54 (45-78) | .08 |
| Platelet count, | 143 (101- | 178 (107- | 126 (101- | .82 |
| ×10³/mL | 231) | 239) | 206) | |
| Use of induction | 18 (47%) | 15 (71%) | 3 (18%) | .001 |
| therapy, n (%) | | | | |

TABLE 1C:
FPS derivation set 3 (NAFLD, cross-sectional, Japan), n = 31

| | | | | |
|---|---|---|---|---|
| Age, y | 49 (31-74) | — | — | — |
| Male sex, n (%) | 24 (77%) | — | — | — |
| BMI, kg/m² | 26.1 (24.5- | — | — | — |
| | 28.6) | | | |
| AST, IU/L | 31 (17-58) | — | — | — |
| ALT, IU/L | 49 (19-139) | — | — | — |
| Platelet count, | 229 (171- | — | — | — |
| ×10³/mL | 386) | | | |

TABLE 1D: FPS derivation set 4 (NAFLD, cross-sectional, U.S.), n = 309

| | | | | |
|---|---|---|---|---|
| Age, y | 44 (36-53) | — | — | — |
| Male sex, n (%) | 75 (25%) | — | — | — |
| BMI, kg/m² | 35.0 (29.2 | — | — | — |
| | 42.1) | | | |
| AST, IU/L | 19 (14-26) | — | — | — |
| ALT, IU/L | 22 (16-33) | — | — | — |
| Platelet count, | 263 (211- | — | — | — |
| ×10³/mL | 311) | | | |

TABLE 1E: FPS validation set 1 (NASH, case-control, Japan),
n = 78 (case:control = 18:60)

| | | | | |
|---|---|---|---|---|
| Age, y | 60 (51-72) | 63 (46-72) | 60 (51-72) | .78 |
| Male sex, n (%) | 44 (56%) | 11 (61%) | 33 (55.%) | .79 |
| BMI, kg/m² | 27.0 | 27.0 (25.0- | 27.4 (25.0- | .90 |
| | (25.0-30.0) | 28.7) | 30.0) | |
| Diabetes, n (%) | 28 (36%) | 4 (22%) | 24 (40%) | .26 |
| Total bilirubin, | 1.0 (0.7-1.1) | 1.0 (0.8-1.2) | 0.8 (0.7-1.1) | .18 |
| mg/dL | | | | |
| Albumin, g/dL | 5.0 (4.3-4.9) | 5.0 (4.3-4.9) | 4.6 (4.3-4.9) | .85 |
| AST, IU/L | 46.0 (30.0- | 42.0 (32.5- | 49.5 (30.0- | .41 |
| | 70.8) | 55.2) | 70.8) | |
| ALT, IU/L | 80.0 (52.2- | 78.0 (56.5- | 80.0 (52.2- | .87 |
| | 108.8) | 97.5) | 108.8) | |
| Platelet count, | 218.0 (195.5- | 232.0 (201.2- | 217.5 (195.5- | .53 |
| ×10³/mL | 262.5) | 271.8) | 262.5) | |
| NASH activity | 4.0 (3.0-5.0) | 4.0 (3.2-5.8) | 4.0 (3.0-5.0) | .32 |
| score (NAS) | | | | |
| Fibrosis stage | 17/36/25 | 12/5/1 | 5/31/24 | <.001 |
| (F1/F2/F3), n (%) | (22%/46%/ | (67%/ | (8%/52%/ | |
| | 32%) | 28%/5%) | 40%) | |
| Follow-up time, y | 2.4 (2.2-3.0) | 2.4 (1.6-2.7) | 3.0 (2.3-3.0) | .33 |

TABLE 1F: FPS validation set 2 (Chronic liver diseases from
various etiologies, U.S.), n = 78

| | | | | |
|---|---|---|---|---|
| Male sex, n (%) | 47 (60%) | — | — | — |
| Etiology | 24/24/3/21 | — | — | — |
| (HCV/HBV/ | (31%/31%/ | | | |
| ALD/NAFLD- | 4%/27%) | | | |
| cryptogenic), | | | | |
| n (%) | | | | |
| Fibrosis stage | 22/22/24/7 | — | — | — |
| (F01/F2/F3/F4), | (29%/29%/ | | | |
| n (%) | 32%/9%) | | | |

TABLE 1G: FPS validation set 3 (NASH, phase IIb CENTAUR
clinical trial, U.S.), n = 19 (treatment:placebo = 9:10)

| | | | | |
|---|---|---|---|---|
| Age, y | 55 (47-60) | 57 (51-58) | 55 (46-62) | .97 |
| Male sex, n (%) | 8 (42%) | 5 (56%) | 3 (30%) | .37 |
| BMI, kg/m² | 36.4 (34.2- | 37.9 (35.2- | 34.4 (28.7- | .40 |
| | 40.2) | 39.8) | 42.9) | |
| Total bilirubin, | 0.7 (0.5-0.9) | 0.6 (0.4-0.7) | 0.8 (0.6-1.1) | .33 |
| mg/dL | | | | |
| Albumin, g/dL | 4.1 (4.0-4.2) | 4.1 (4.0-4.2) | 4.1 (3.9-4.3) | .93 |
| AST, IU/L | 40.9 (30.6- | 34.2 (29.9- | 47.6 (36.4- | .97 |
| | 56.4) | 62.6) | 55.9) | |
| ALT, IU/L | 48.2 (41.8- | 48.9 (45.6- | 47.1 (40.5- | .60 |
| | 55.7) | 57.5) | 50.2) | |
| Platelet count, | 237.0 (200.0- | 237.0 (198.0- | 233.5 (205.0- | .71 |
| ×10³/mL | 256.5) | 250.0) | 284.5) | |
| NASH activity | 5.0 (4.0-6.0) | 6.0 (5.0-6.0) | 5.0 (4.0-5.8) | .41 |
| score (NAS) | | | | |
| Fibrosis stage | 2/9/8 | 2/4/3 | 0/5/5 | .46 |
| (F1/F2/F3), n (%) | (11%/47%/ | (22%/44%/ | (0%/50%/ | |
| | 42%) | 33%) | 50%) | |

TABLE 1H: FPSec validation set (compensated cirrhosis with mixed
etiologies, cohort, U.S.), n = 122

| | | | | |
|---|---|---|---|---|
| Age, y | 51 (46-57) | — | — | — |
| Male sex, n (%) | 80 (66%) | — | — | — |
| Total bilirubin, | 0.9 (0.6-1.2) | — | — | — |
| mg/dL | | | | |
| Albumin, g/dL | 3.8 (3.6-4.1) | — | — | — |
| AST, IU/L | 53 (35-74) | — | — | — |
| ALT, IU/L | 50 (34-75) | — | — | — |
| Platelet count, | 116 (82-160) | — | — | — |
| ×10³/mL | | | | |

TABLE 1I: PLS validation set 1, FPS derivation set 1
(chronic hepatitis C, case-control, U.S.)

| | | | | p- |
|---|---|---|---|---|
| Variable | All cases | Cases | Controls | value |
| No. patients | 43 | 12 | 31 | — |
| Age, y | 46 (42-49) | 48 (45-49) | 45 (41-50) | 0.212 |
| Male sex, n (%) | 35 (81%) | 11 (92%) | 24 (77%) | 0.407 |
| Total bilirubin, | 0.6 (0.4-1.0) | 0.6 (0.4-0.9) | 0.6 (0.4-1.0) | 0.796 |
| mg/dL | | | | |
| Albumin, g/dL | 4.0 (3.8-4.3) | 4.0 (3.7-4.2) | 4 (3.8-4.3) | 0.673 |
| AST, IU/L | 41 (30.2- | 69 (50.0- | 35 (29.5- | 0.004 |
| | 66.8) | 75.5) | 45.0) | |
| ALT, IU/L | 42 (31.5- | 59.5 (46.0- | 36 (30.0- | 0.011 |
| | 64.5) | 92.5) | 54.0) | |
| ALP, IU/L | 83 (72.0- | 93 (79.0- | 82 (71.2- | 0.23 |
| | 95.0) | 95.5) | 93.8) | |
| Platelet count, | 218 (185.0- | 193.5 (177.8- | 228 (192.5- | 0.044 |
| ×10³/mL | 249.0) | 219.2) | 255.0) | |
| HCV-RNA, | 18 (42%) | 6 (50%) | 12 (39%) | 0.711 |
| ×10³/mL | | | | |
| HIV co-infection, | 24 (56%) | 9 (75%) | 15 (48%) | 0.17 |
| n (%) | | | | |
| CD4 < 400 /mL* | 13 (30%) | 3 (25%) | 10 (32%) | 0.206 |
| Follow-up time, y | 5.8 (4.3-8.8) | 2.1 (1.9-4.2) | 7.2 (5.7-9.5) | |

TABLE 1J: PLS validation set 2,
FPS derivation set 2 (transplantation, case-control, U.S.)
*In HIV-co-infected patients.

| | | | | |
|---|---|---|---|---|
| No. patients | 38 | 21 | 17 | — |
| Donor/recipient | 42 (23-47)/52 | 28 (23-50)/ | 42 (37-46)/ | 0.27/ |
| age, y | (46-55) | 54 (50-53) | 47 (44-52) | 0.02 |
| Male sex, n (%) | 25 (66%) | 11 (52%) | 14 (82%) | 0.09 |
| BMI, kg/m² | 28.8 (24.2- | 29.2 (24.2- | 28.8 (24.9- | 0.78 |
| | 34.0) | 33.5) | 34.0) | |
| Diabetes-pre- | 8 (21%) | 3 (14%) | 5 (29%) | 0.43 |
| transplant, n (%) | | | | |
| Diabetes-1 | 14 (37%) | 5 (24%) | 9 (53%) | 0.09 |
| year post- | | | | |
| transplant, n (%) | | | | |
| Total bilirubin, | 0.7 (0.4-0.9) | 0.8 (0.5-0.9) | 0.5 (0.3-0.8) | 0.09 |
| mg/dL | | | | |
| Albumin, g/dL | 4.0 (3.7-4.1) | 3.9 (3.7-4.1) | 4.0 (3.9-4.2) | 0.26 |
| AST, IU/L | 55 (37-112) | 71 (46-177) | 39 (33-58) | 0.02 |
| ALT, IU/L | 60 (46-115) | 78 (48-206) | 54 (45-78) | 0.08 |
| ALP, IU/L | 154 (118- | 178 (134- | 147 (99-211) | 0.36 |
| | 225) | 229) | | |
| Platelet count, | 143 (101- | 178 (107- | 126 (101- | 0.82 |
| ×10³/mL | 231) | 239) | 206) | |
| HCV-RNA, | 376 (101- | 443 (111- | 296 (93- | 0.99 |
| ×10³m/L | 936) | 850) | 1170) | |
| Cytomegalovirus | 4 (11%) | 3 (14%) | 1 (6%) | 0.61 |
| infection, n (%) | | | | |
| Use of induction | 18 (47%) | 15 (71%) | 3 (18%) | 0.001 |
| therapy, n (%) | | | | |
| Use of tacrolimus | 23 (61%) | 11 (52%) | 12 (71%) | 0.44 |
| (vs. CsA), n (%) | | | | |
| Ischemia time, hr | 7.2 (5.8-9.2) | 8.7 (5.7-9.4) | 6.6 (5.9-8.3) | 0.37 |
| Follow-up time, y | 4.7 (2.8-6.0) | 3.9 (2.0-5.0) | 6.2 (5.0-8.9) | — |

TABLE 1K: FPS derivation set 3 (NAFLD, cross-sectional, Japan)

| | | | | |
|---|---|---|---|---|
| No. patients | 31 | — | — | — |
| Age, y | 49 (31-74) | — | — | — |
| Male sex, n (%) | 24 (77%) | — | — | — |
| AST, IU/L | 31 (17-58) | — | — | — |
| ALT, IU/L | 49 (19-139) | — | — | — |
| Platelet count, | 229 (171- | — | — | — |
| ×10³/mL | 386) | | | |
| Diabetes mellitus | 15 (48%) | — | — | — |
| BMI, kg/m² | 26 (20-39) | — | — | — |
| NASH activity | 4.0 (3.0-5.0) | — | — | — |
| score (NAS) | | | | |

TABLE 1L: FPS derivation set 4 (NAFLD, cross-sectional, U.S.)

| | | | | |
|---|---|---|---|---|
| No. patients | 309 | — | — | — |
| Age, y | 44 (36-53) | — | — | — |
| Male sex, n (%) | 75 (25%) | — | — | — |
| AST, IU/L | 19 (14-26) | — | — | — |
| ALT, IU/L | 22 (16-33) | — | — | — |
| Platelet count, | 263 (211- | — | — | — |
| ×10³/mL | 311) | | | |
| Diabetes mellitus | 43 (20%) | — | — | — |
| BMI, kg/m² | 30 (26-36) | — | — | — |
| NASH activity | 2.0 (1.0-3.0) | — | — | — |
| score (NAS) | | | | |

TABLE 1M: FPS validation set 1 (NASH, case-control, Japan)
*In HIV-co-infected patients.

| | | | | |
|---|---|---|---|---|
| No. patients | 78 | 18 | 60 | — |
| Age, y | 60 (51-72) | 63 (46-72) | 60 (51-72) | 0.78 |
| Male sex, n (%) | 44 (56%) | 11 (61%) | 33 (55.%) | 0.788 |
| BMI, kg/m² | 27.0 (25.0- | 27.0 (25.0- | 27.4 (25.0- | 0.901 |
| | 30.0) | 28.7) | 30.0) | |
| Diabetes, n (%) | 28 (36%) | 4 (22%) | 24 (40%) | 0.263 |
| NASH activity | 4.0 (3.0-5.0) | 4.0 (3.2-5.8) | 4.0 (3.0-5.0) | 0.323 |
| score (NAS) | | | | |
| Fibrosis stage | 17/36/25 | 12/5/1 | 5/31/24 | <0.001 |
| (F1/F2/F3), n (%) | (22%/46%/ | (67%/ | (8%/52%/ | |
| | 32%) | 28%/5% | 40% | |
| Platelet count, | 218.0 (195.5- | 232.0 (201.2- | 217.5 (195.5- | 0.53 |
| ×10³/mL | 262.5) | 271.8) | 262.5) | |
| Total bilirubin, | 1.0 (0.7-1.1) | 1.0 (0.8-1.2) | 0.8 (0.7-1.1) | 0.183 |
| mg/dL | | | | |
| Albumin, g/dL | 5.0 (4.3-4.9) | 5.0 (4.3-4.9) | 4.6 (4.3-4.9) | 0.854 |
| AST, IU/L | 46.0 (30.0- | 42.0 (32.5- | 49.5 (30.0- | 0.41 |
| | 70.8) | 55.2) | 70.8) | |
| ALT, IU/L | 80.0 (52.2- | 78.0 (56.5- | 80.0 (52.2- | 0.873 |
| | 108.8) | 97.5) | 108.8) | |
| Follow-up time, y | 2.4 (2.2-3.0) | 2.4 (1.6-2.7) | 3.0 (2.3-3.0) | 0.334 |

TABLE N: FPS validation set 2 (Chronic liver
diseases from various etiologies, U.S.)

| | | | | |
|---|---|---|---|---|
| No. patients | 78 | — | — | — |
| Male sex, n (%) | 47 (60%) | — | — | — |
| Etiology | 24/24/3/21 | | | |
| (HCV/HBV/ | (31%/31%/ | | | |
| ALD/NAFLD- | 4%/27% | | | |
| cryptogenic), | | | | |
| n (%) | | | | |
| Fibrosis stage | 22/22/24/7 | — | — | — |
| (F01/F2/F3/F4), | (29%/29%/ | | | |
| n (%) | 32%/9% | | | |

TABLE 1O: FPS validation set 3 (NASH, phase
IIb CENTAUR clinical trial, U.S.)

| | | | | |
|---|---|---|---|---|
| No. patients | 19 | 9 (treatment | 10 (placebo | — |
| | | arm) | arm) | |
| Age, y | 55 (47-60) | 57 (51-58) | 55 (46-62) | 0.97 |
| Male sex, n (%) | 8 (42%) | 5 (56%) | 3 (30%) | 0.37 |
| BMI, kg/m² | 36.4 (34.2- | 37.9 (35.2- | 34.4 (29.0- | 0.4 |
| | 40.2) | 39.8) | 41.9) | |
| Diabetes, n (%) | 8 (42%) | 6 (67%) | 2 (20%) | 0.07 |
| NASH activity | 5.0 (4.0-6.0) | 6.0 (5.0-6.0) | 5.0 (4.0-5.8) | 0.41 |
| score (NAS) | | | | |
| Fibrosis stage | 2/9/8 | 2/4/3 | 0/5/5 | 0.459 |
| (F1/F2/F3), n (%) | (11%/47%/ | (22%/44%/ | (0%/50%/ | |
| | 42%) | 33% | 50%) | |
| Platelet count, | 237.0 (200.0- | 237.0 (198.0- | 233.5 (205.0- | 0.71 |
| ×10³/mL | 256.5) | 250.0) | 284.5) | |
| Total bilirubin, | 0.7 (0.5-0.9) | 0.6 (0.4-0.7) | 0.8 (0.6-1.1) | 0.33 |
| mg/dL | | | | |
| Albumin, g/dL | 4.1 (4.0-4.2) | 4.1 (4.0-4.2) | 4.1 (3.9-4.3) | 0.93 |
| AST, IU/L | 40.9 (30.6- | 34.2 (29.9- | 47.6 (36.4- | 0.97 |
| | 56.4) | 62.6) | 55.9) | |
| ALT, IU/L | 48.2 (41.8- | 48.9 (45.6- | 47.1 (40.5- | 0.6 |
| | 55.7) | 57.5) | 50.2) | |

Categorical and continuous (shown as median and IQR) variables are compared by Fisher's exact test and Wilcoxon rank-sum test, respectively.
AST, aspartate aminotransferase;
ALT, alanine aminotransferase;
ALP, alkaline phosphatase;
BMI, body mass index;
CsA, cyclosporine A.

### Ex Vivo and In Vitro Assessment of Pharmacological Effects of Candidate Anti-Fibrotic Agents

Evaluation of galunisertib, erlotinib, AM095, bortezomib, pioglitazone, metformin, epigallocatechin gallate (EGCG), I-BET 151, JQ1, captopril, nizatidine (Selleck Chemicals); MG-132 (Sigma-Aldrich); cenicriviroc (AbbVie) (Table 2) were done in organotypic ex vivo culture of precision-cut liver slice (PCLS) tissues in the FPS validation set 2 as previously described. Patient-derived liver cell spheroids were generated from a cirrhosis patient and high-risk FPS was induced by free fatty acids, and treated with EGCG, bortezomib, cenicriviroc, and/or bezafibrate for 48 h. Mycoplasma-free human hepatic myofibroblast cell lines, LX-2 and TWNT-4, were cultured with MG-132 (20 μM) or DMSO control for 12 and 24 h in triplicates.

TABLE 2
Compounds tested in organotypic ex vivo precision-cut liver
slice (PCLS) and liver spheroid culture.

| | | | | |
|---|---|---|---|---|
| | | | | No. |
| Compound | | Anticipated | Concen- | cultured |
| name | Compound class | targets | tration | cases |

TABLE 2A: PCLS culture

| | | | | |
|---|---|---|---|---|
| Galunisertib | Cellular signaling | TGF-ß | 10 μM | 13 |
| | receptor inhibitor | receptor 1 | | |
| Erlotinib | Cellular signaling | EGF | 5 μM | 25 |
| | receptor inhibitor | receptor | | |
| AM095 | Cellular signaling | LPA | 3 μM | 29 |
| | receptor inhibitor | receptor 1 | | |
| MG132 | Proteasome | BCL2 | 20 μM | 8 |
| | inhibitor; CMap- | | | |
| | derived BCL2 | | | |
| | antagonist | | | |
| Bortezomib | Proteasome | BCL2 | 100 μM | 7 |
| | inhibitor; CMap- | | | |
| | derived BCL2 | | | |
| | antagonist | | | |
| Cenicriviroc | Chemokine | CCR2/ | 100 nM | 13 |
| | receptor | CCR5 | | |
| | inhibitor | | | |
| Pioglitazone | Anti-diabetic | PPAR-γ | 20 μM | 10 |
| Metformin | Anti-diabetic | AMPK | 5 μM | 8 |
| Epigall- | Dietary | 67-kDa | 30 μM | 11 |
| ocatechin | nutrituional | laminin | | |
| gallate | substance; | receptor | | |
| (EGCG) | green tea catechin | | | |
| I-BET 151 | BET | BRD2, | 1 μM | 2 |
| | bromodomain | BRD3, | | |
| | inhibitor | BRD4 | | |
| JQ1 | BET | BRD4 | 1 μM | 2 |
| | bromodomain | | | |
| | inhibitor | | | |
| Captopril | ACE inhibitor; | ACE | 100 μM | 3 |
| | CMap-derived | | | |
| | PLS antagonist | | | |
| Nizatidine | Anti-histamine; | Histamine | 10 μM | 6 |
| | CMap-derived | H2 | | |
| | PLS antagonist | receptor | | |

TABLE 2B: Spheroid culture

| | | | | |
|---|---|---|---|---|
| EGCG | Dietary nutritional | 67-kDa | 20 μM | 1 |
| | substance; green | laminin | | |
| | tea catechin | receptor | | |
| Bortezomib | Proteasome | BCL2 | 50 μM | 1 |
| | inhibitor; CMap- | | | |
| | derived BCL2 | | | |
| | antagonist | | | |
| Cenicriviroc | Chemokine receptor | CCR2/ | 50 nM | 1 |
| | inhibitor | CCR5 | | |
| Bezafibrate | Proliferator- | PPARa | 100 μM | 1 |
| | activated receptor | | | |
| | (PPAR) alpha | | | |
| | agonist | | | |

CMap: Connectivity Map database (https://clue.io/cmap).

### Immunostaining

Immunostaining was performed for caspase-3 (Asp175) (5A1E, Cell Signaling), alpha-SMA (Abcam), Desmin (DAKO), GFAP (abcam), and Ki-67 (Abcam). TUNEL staining was performed using ApopTag Peroxidase In Situ Apoptosis Detection Kit (EMD Millipore).

### Gene and Protein Expression Profiling

Total RNA was isolated from fixed tissue sections by using High Pure RNA Paraffin kit (Roche), and assessed for quality by qRT-PCR of RPL13A. Total RNA from the PCLS tissues was isolated using RNeasy kit (Qiagen). RNA samples (100-200 ng) were subjected to the PLS/FPS assay implemented in the nCounter platform (NanoString), and transcriptome profiling of the CENTAUR trial samples was performed by RNA-Seq (TrueSeq RNA Access, Illumina). Expression of BCL2, COL1A1, and ACTA2 genes was measured by qRT-PCR (Table 3). Serum protein profiling was performed by using xMAP assay (Luminex).

TABLE 3
PCR primer sequences.

| | Entrez | | |
|---|---|---|---|
| | gene | Forward (5′-3′) | Reverse (5′-3′) |
| Gene | ID | (SEQ ID NO) | (SEQ ID NO) |
| COL1A1 | 1277 | GTACTGGATTGA | CGCCATACTCGAA |
| | | CCCCAACC | CTGGAAT |
| | | (SEQ ID NO: 1) | (SEQ ID NO: 2) |
| ACTA2 | 59 | CCCCATCTATGA | CAGTGGCCATCTC |
| | | GGGCTATG | ATTTTCA |
| | | (SEQ ID NO: 3) | (SEQ ID NO: 4) |
| BCL2 | 596 | TCGCCCTGTGGA | CAGAGACAGCCAG |
| | | TGACTGA | GAGAAATCA |
| | | (SEQ ID NO: 5) | (SEQ ID NO: 6) |
| RPL13A | 23521 | GTACGCTGTGAA | GTTGGTGTTCATC |
| | | GGCATCAA | CGCTTG |
| | | (SEQ ID NO: 7) | (SEQ ID NO: 8) |

### Gene Expression Profiling

Total RNA was isolated from three to five 10-μm-thick formalin-fixed paraffin-embedded (FFPE) tissue sections from the PLS/FPS derivation/validation cohorts by using High Pure RNA Paraffin kit (Roche), and lack of severe RNA fragmentation was confirmed by qRT-PCR of a housekeeping gene RPL13A as previously described.¹ From freshly harvested myofibroblast cell lines, LX-2 and TWNT-4, and clinical precision-cut-liver slice (PCLS) tissues stored in RNAlater (ThermoFisher) at −80?C, total RNA was isolated using RNeasy kit (Qiagen), and the RNA integrity number (RIN)>8 by Bioanalyzer (Agilent) was regarded as sufficient quality for expression analysis. Total RNA samples (100 to 500 ng) were subjected to the gene signature assays implemented in the digital transcript counting technology (NanoString) according to manufacturer's instruction. Poor quality profiles were detected based on maximum signal intensity from positive control probes <3,000U. Raw transcript count data were log-transformed (base 2) and scaled by geometric mean of control probe data by using NanoString normalizer module implemented in GenePattern data analysis suite (www.broadinstitute.org/genepattern). Genome-wide transcriptome profiling of the CENTAUR trial samples was performed using 100 to 200 ng total RNA by RNA-Seq using exome-enriched library preparation according to the manufacturer's protocol (TrueSeq, Illumina). Raw sequencing reads were mapped onto the reference human genome (hg19) by using the STAR aligner (ver. 2.6.1b) followed by the read counting on genes via featureCounts in the Subread package (ver. 1.6.1). The raw read counts were further normalized by using the Relative Log Expression (RLE) implemented in the DESeq2 package (ver. 1.22.2). Expression of COL1A1, and ACTA2, and BCL2 genes was measured by qRT-PCR (BioRad) using ddCt method with RPL13A as housekeeping gene, as previously described (refer Table 3 for the primer sequences). Gene expression profiles of the PLS/FPS-inducible cell culture model (cell-culture-derived PLS [cPLS] system) treated with erlotinib, pioglitazone, captopril, and resveratrol were obtained from our previous study (GSE81801).

### Serum Protein Profiling

Serum-protein-based surrogate of the FPS was determined as the Fibrosis Progression Secretome signature (FPSec) (Table 4) from the FPS member genes by using our computational pipeline, Translation of tissue gene expression to secretome (TexSEC, www.texsec-app.org), and implemented in xMAP assay (Luminex). Seventy μL of serum samples stored at −80?C were spun immediately before running the assay to remove debris, and subjected to protein abundance profiling on the Bio-Plex 200 systems (Bio-Rad) at UT Southwestern BioCenter as previously described.

TABLE 4
Correlation between tissue mRNA and serum protein expression
in the FPSec optimization set (n = 79).

| | Serum protein (FPSec | Correlation | False discovery |
|---|---|---|---|
| Tissue mRNA | panel) | coefficient | rate |
| TTR | ANG | 0.68 | <.001 |
| AEBP1 | MMP7 | 0.61 | <.001 |
| AEBP1 | IGFBP7 | 0.60 | <.001 |
| KRT7 | MMP7 | 0.55 | <.001 |
| F9 | PROS1 | 0.52 | <.001 |
| ANXA1 | IGFBP7 | 0.52 | <.001 |
| CXCR4 | VCAM1 | 0.47 | <.001 |
| FBN1 | IGFBP7 | 0.46 | <.001 |
| PON3 | ANG | 0.46 | <.001 |
| BCL2 | MMP7 | 0.45 | <.001 |
| CXCR4 | IGFBP7 | 0.45 | <.001 |
| FILIP1L | IL6 | 0.44 | <.001 |
| ANXA1 | VCAM1 | 0.44 | <.001 |
| IER3 | PROS1 | −0.43 | <.001 |
| HAAO | IGFBP7 | −0.48 | <.001 |
| AEBP1 | PROS1 | −0.50 | <.001 |
| HAAO | MMP7 | −0.57 | <.001 |
| BCL2 | PROS1 | −0.63 | <.001 |

With CCL21, which is already a member of FPS, the FPSec panel includes 5 high-risk proteins (VCAM1, IGFBP7, MMP7, IL6, CCL21) and 2 low-risk proteins (PROS1, ANG).

### Bioinformatic and Statistical Data Analysis

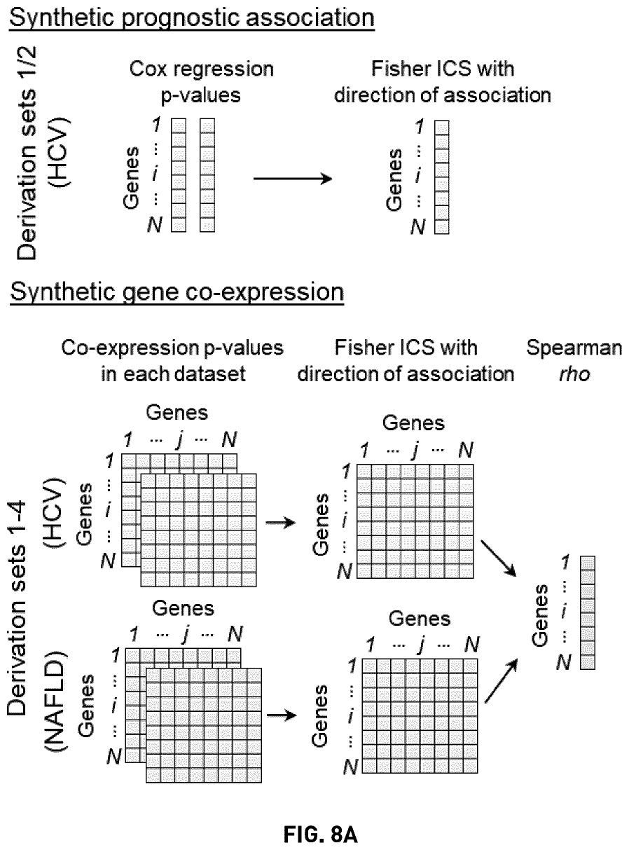

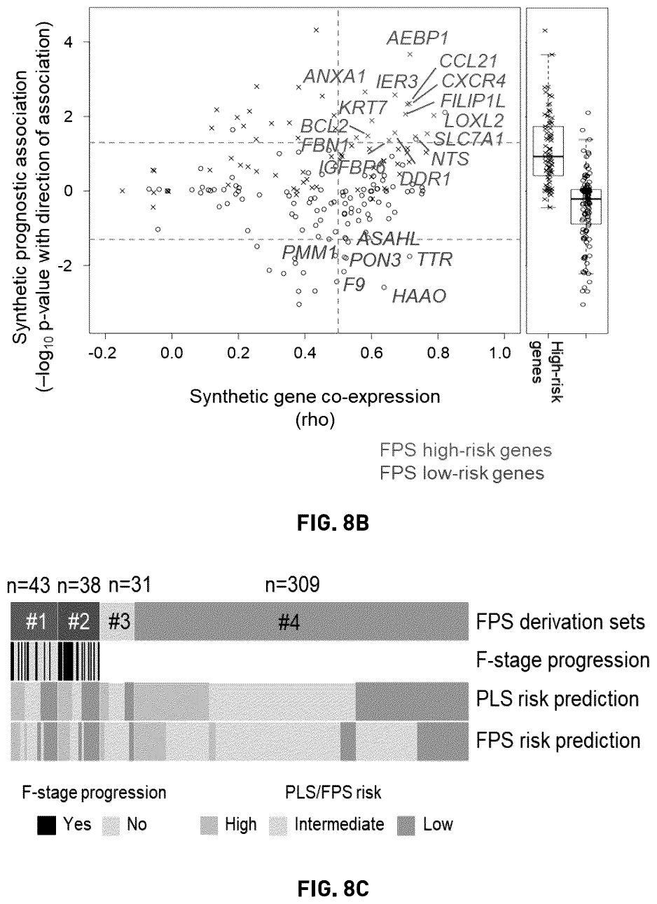

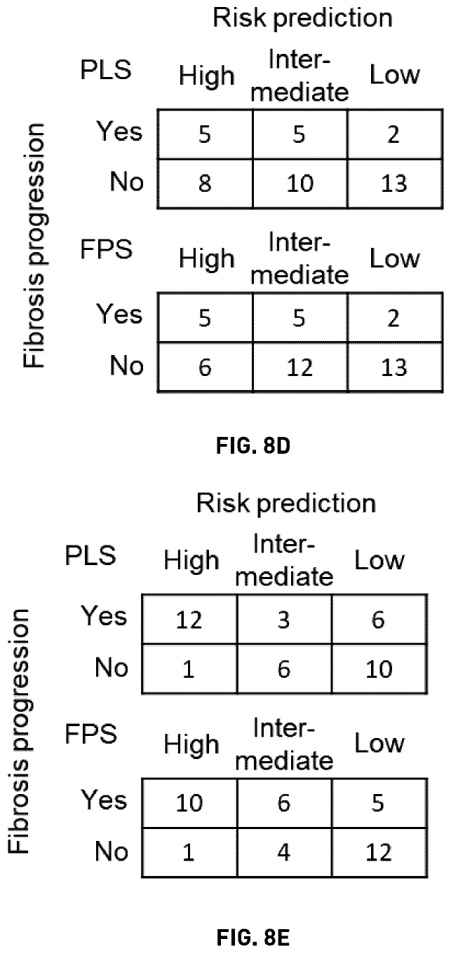

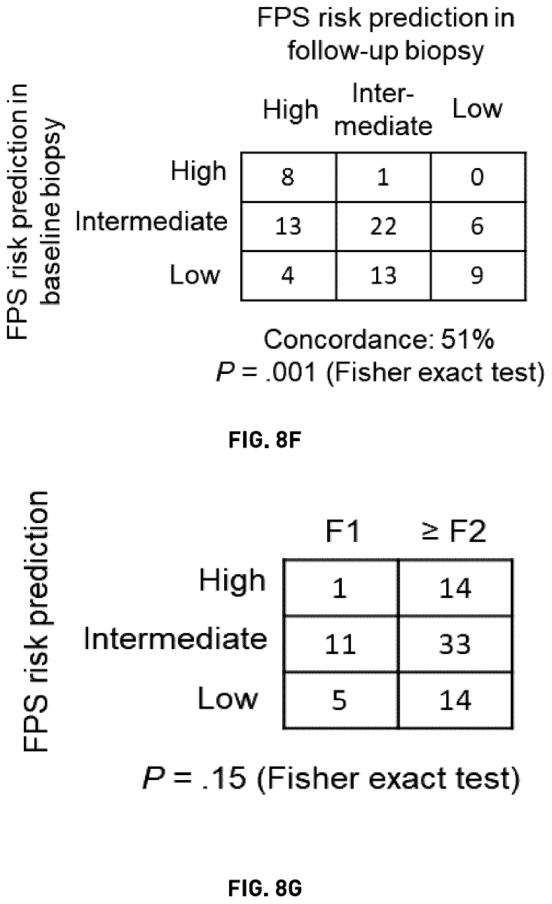

FPS was defined as a subset of PLS specifically associated with time to fibrosis progression and with shared transcriptional regulation between viral (HCV, n=81) and metabolic (NAFLD, n=340) etiologies using Fisher's inverse chi-square statistic (FIG. 8). Prognostic prediction was performed by using Nearest Template Prediction algorithm, and association with the clinical outcome was evaluated with uni/multivariable logistic regression. Modulation of gene signatures and molecular pathways was assessed by gene set enrichment index (GSEI), and co-expression gene networks were defined by MEGENA. Rational combination anti-fibrotic therapies were computationally explored based on combinatorial enhanced modulation of the FPS from high- to low-risk pattern in the transcriptome data of the clinical PCLS tissues cultured with the candidate anti-fibrotic agents. Datasets are available at NCBI Gene Expression Omnibus (GSE85550).

### Molecular-Signature-Based Prognostic Prediction.

The gene/protein-signature-based clinical outcome prediction was performed based on previously reported Nearest Template Prediction (NTP)11 model and a prediction of poor, intermediate, and good prognosis was determined based on prediction confidence p<0.05 as previously reported. Association of the prognostic prediction and clinical outcome was evaluated by uni- and multivariable logistic regression modeling. Clinical variables with univariable p<0.10 were included in the multivariable modeling using stepwise variable selection based on Akaike's Information Criterion.

### Modulation of Gene Signatures and Molecular Pathways.

Modulation (i.e., induction or suppression) of the prognostic gene signatures (i.e., PLS, FPS, and FPSec), molecular pathway gene sets from the Molecular Signature Database (MSigDB ver. 7, www.gsea-msigdb.org/gsea/msigdb) and transcriptome signatures of hepatic stellate cells (HSC)/myofibroblasts for their presence and activation status was assessed by Gene Set Enrichment Analysis (GSEA) for sample-group-based analysis or modified GSEA for individual-sample- or paired-sample-based assessment (GenePattern eseach and PairedEseach modules, gparc.org) of gene set enrichment, and visualized as the Gene Set Enrichment Index (GSEI) as previously described. Transcriptional target gene signatures for key fibrosis/myofibroblast regulator genes, e.g., PDGFRB, were derived from CRISPR- and shRNA-based genetic perturbation transcriptome signature database, iLINCS (www.ilincs.org/ilincs). For bi-directional prognostic gene signatures (i.e., gene signatures, including both poor- and good-prognosis-associated genes), both gene set enrichment score (ES) for each subcomponent of the signature and combined enrichment score (CES) defined as poor-prognosis genes' ES minus good-prognosis genes' ES were calculated.

### Co-Expression Gene Networks.

Co-expression gene network analysis was performed and hub genes (or key driver genes) in the networks were identified by Multiscale Embedded Gene Co-expression Network Analysis (MEGENA) algorithm, synthesizing multiple patient cohorts by using the Fisher's inverse chi-square statistic. Association of gene expression with time to censored clinical outcome information was assessed by the Cox score using GenePattern SurvivalGene module (gparc.org).

### Derivation of Fibrosis Progression Signature (FPS).

The FPS genes were defined as a subset of the Prognostic Liver Signature (PLS), which are more specifically associated with fibrosis progression and share similar regulation between viral (HCV; n=81) and metabolic (NAFLD; n=340) etiologies, by integrating the FPS derivation sets 1 to 4 (FIG. 1, Table 1). First, association of each gene's expression with time to fibrosis progression was calculated in each of the FPS derivation sets 1 and 2 as a Cox score1 and its nominal p-value based on 1,000 random sample permutations. The prognostic association was next synthesized across the cohorts as the prognostic score (for gene i in the datasets) by using a modified Fisher's inverse chi-square statistic16 as follows:

$$Prognostic {score}_{i} = - 2 {\sum }_{j = 1}^{J} sign ( {Cox}_{i j} ) \cdot ln ( nominal {p}_{ij} )$$

where Coxᵢⱼ and nominal pᵢⱼ are Cox score and its nominal p-value, respectively, for the i-th gene in the j-th cohort; and sign(Coxᵢⱼ) denotes positive or negative sign of Coxᵢⱼ

In parallel, shared transcriptional regulation within each of the HCV (i.e., FPS derivation sets 1 and 2) and NAFLD (i.e., FPS derivation sets 3 and 4) cohorts was defined by using correlation p-values for each gene pair across the patient cohorts, which were synthesized as a co-expression score (coexp) as follows:

$${coexp}_{ij} = - 2 {\sum }_{k = 1}^{K} sign ( {corr}_{ijk} ) \cdot ln ( {p}_{ijk} )$$

where corrᵢⱼₖ and pᵢⱼₖ are correlation coefficient and p-value, respectively, between the i-th and the j-th genes in the k-th cohort; sign (corrᵢⱼₖ) denotes positive or negative sign of corrᵢⱼₖ; K is number of cohorts.

Next, for each gene i, similarity of co-expressed genes between HCV and NAFLD cohorts was determined by the following co-expression similarity (coexpSim) score:

coexpSimᵢ=Spearman correlation (HCVcoexpᵢ, NAFLDcoexpᵢ)

where HCVcoexpᵢ and NAFLDcoexpᵢ represent vectors of the co-expression scores between the i-th gene and all the other genes in the HCV and NAFLD cohorts, respectively, and their Spearman correlation p-value was calculated. Finally, genes with their abs (prognostic significance score) greater than 1.301 (corresponding to p<0.05) and coexpSim; greater than 0.5 (confident shared transcriptional regulation) were selected as the FPS member genes.

### Computational Inference of Combination Anti-Fibrotic Therapies.

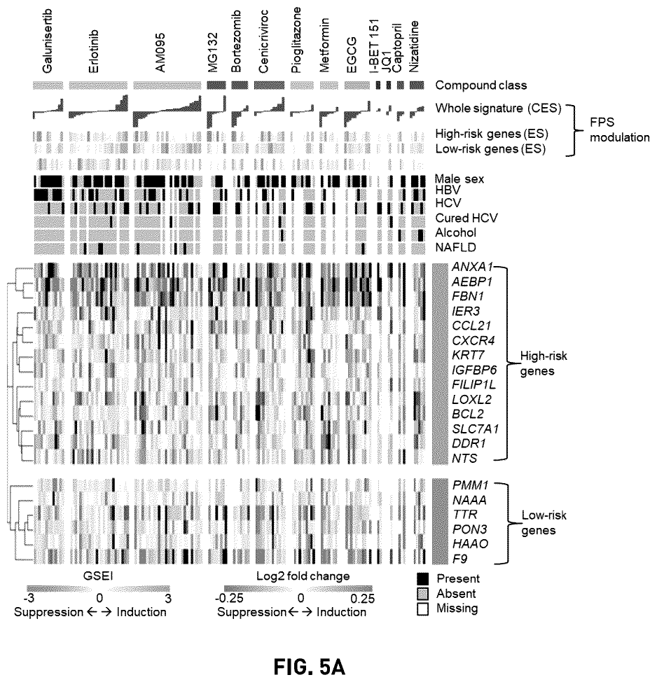

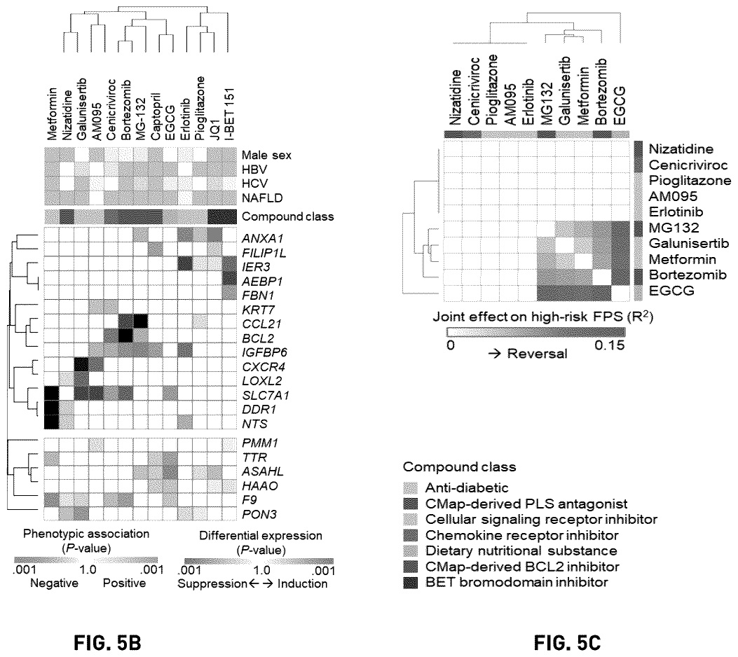

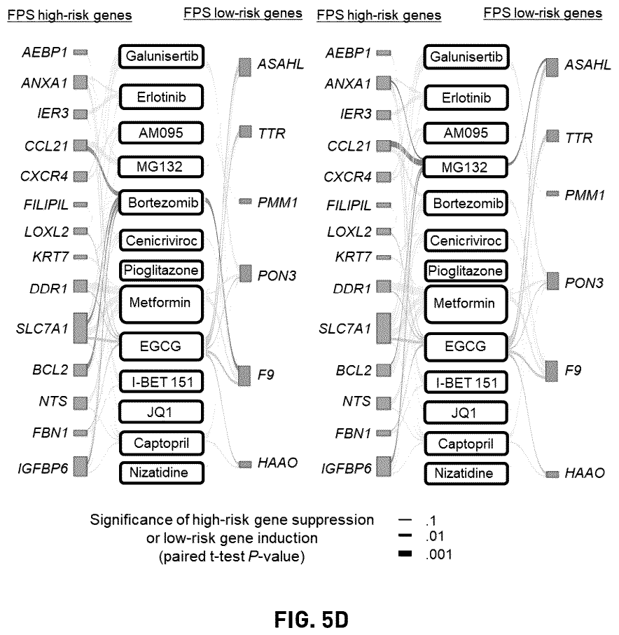

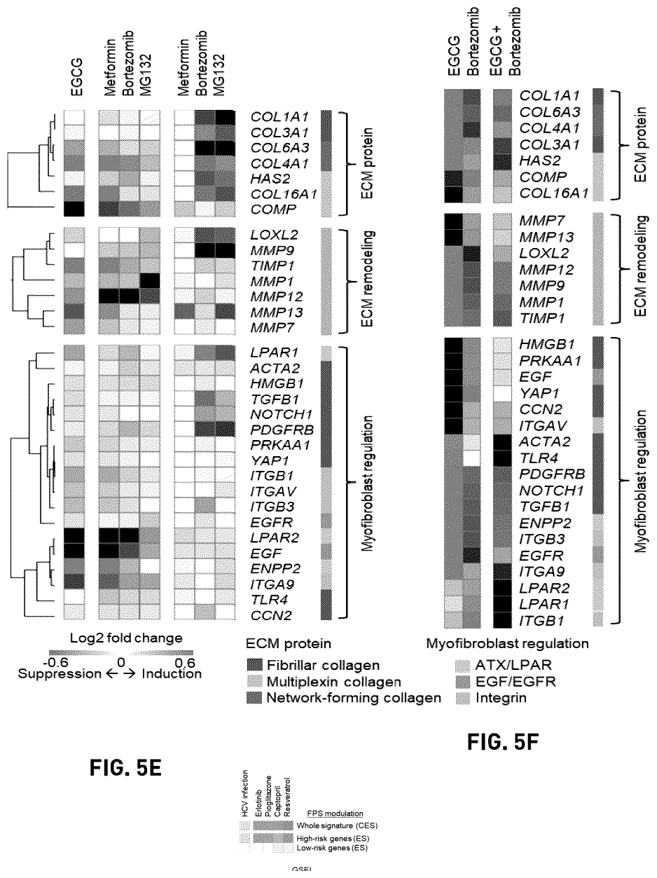

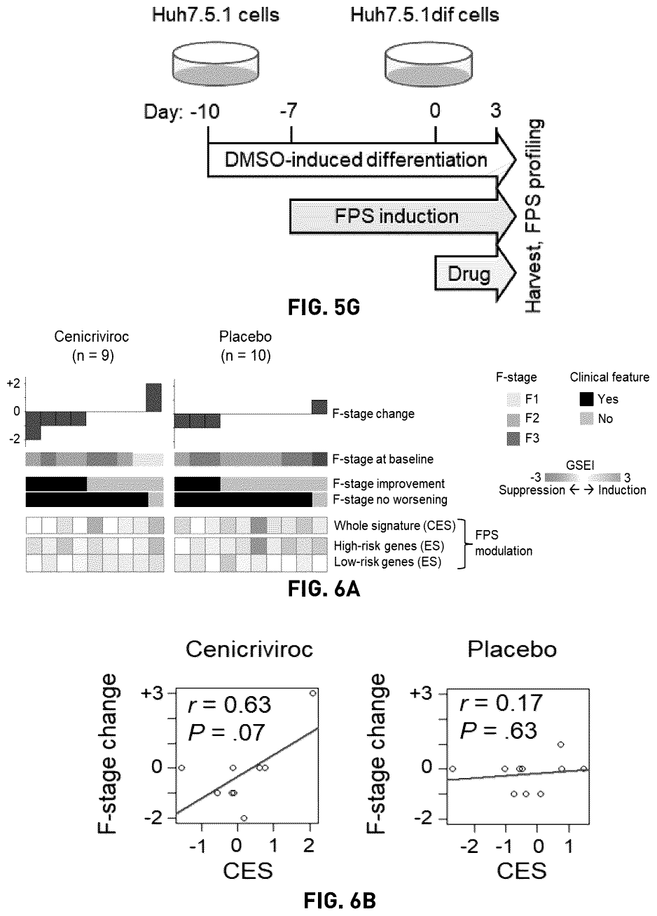

To identify rational combination anti-fibrotic therapies, combinatorial modulation of the FPS from high- to low-risk pattern was assessed using the single-agent FPS response in the ex vivo culture of clinical PCLS tissues (see FIG. 5A). For each of the FPS genes, paired t-test p-value was calculated by comparing compound- and DMSO-treated tissues, which was subsequently transformed into a signed Z-score as a measure of pharmacological modulation on its expression. Sole and combinatorial effects with all pairs of two compounds on inverse association with the prognostic scores (see previous section) of the entire FPS genes were modeled to measure the magnitude of converting a high-risk pattern of FPS into a low-risk pattern by a linear regression as follows:

$$Prognostic {score}_{i}^{′} = - {\sum }_{i}^{C} {β}_{d} {Z}_{d i}$$

where Prognostic score′ᵢ is the inverse transformation of Prognostic scoreᵢ, making it follow the Gaussian distribution and satisfy the assumption for linear regression modeling; C denotes a combination of two compounds; Z_{di} is the signed Z-score for the compound d in C for the i-th gene. β_{d} is regression coefficient (assumed to be ≥0), which was estimated by the non-negative least square method. Squared correlation (R²) between the observed and predicted prognostic risk scores was used as a quantitative measure of converting a high-risk FPS into a low-risk FPS (when positive sign is assigned to R²) or a low-risk FPS into a high-risk FPS (when negative sign is assigned to R²) to rank the compound combinations. Combinations of compounds tested in more than five PCLS tissues (i.e., patients) and with positive R² were visualized in a heatmap.

### Statistical Analysis, Access to the Datasets.

Categorical and continuous variables were compared by Fisher's exact test and Wilcoxon rank-sum test, respectively. Correction for multiple hypothesis testing was performed by using Bonferroni correction or Benjamini-Hochberg false discovery rate (FDR) as appropriate. A two-tailed p-value <0.05 was regarded as statistically significant. All bioinformatic and statistical analyses were performed using R statistical language (www.r-project.org), and all datasets are available at NCBI Gene Expression Omnibus (GSE85550).

### Example 1: PLS is Associated with 5-Year Fibrosis Progression in Chronic Hepatitis C with No or Minimal Fibrosis

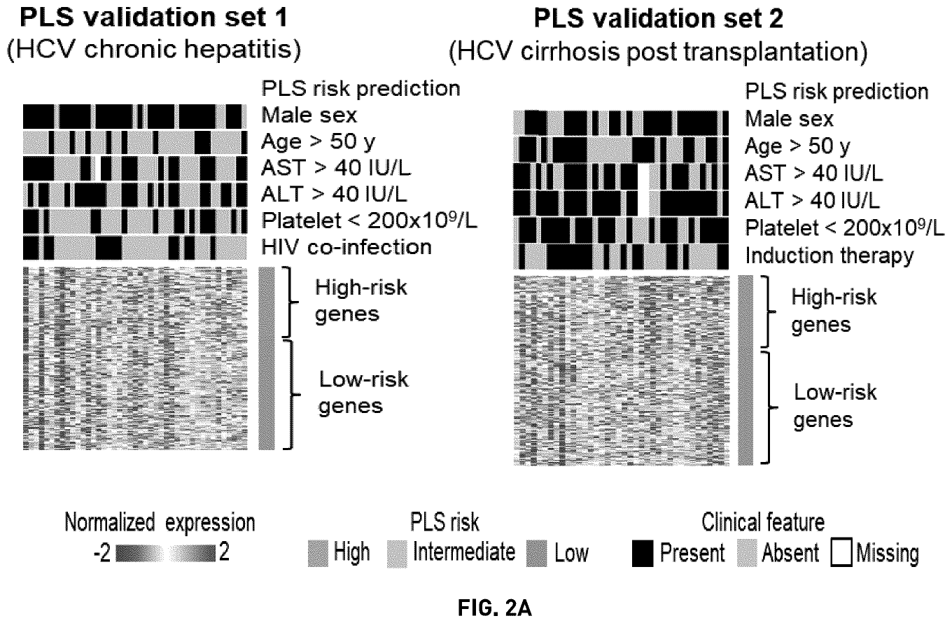

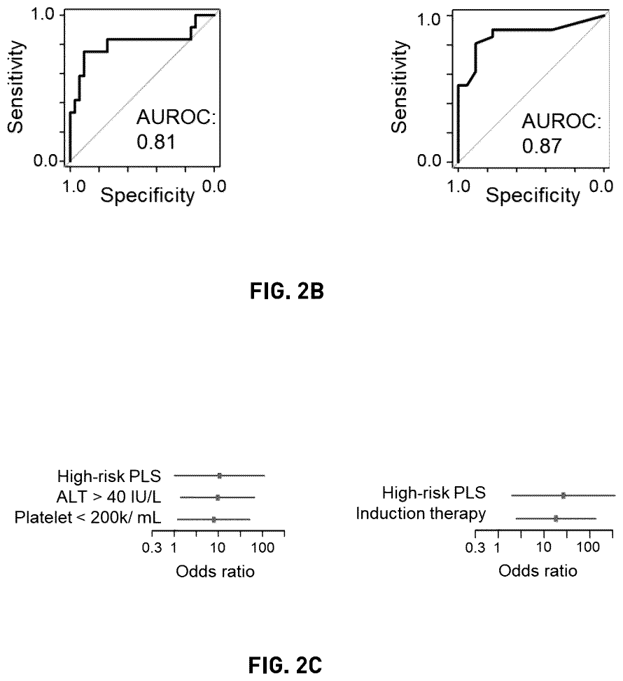

To clarify whether a hepatic transcriptome signature (described in U.S. application Ser. No. 17/896,944 which is incorporated herein by reference in its entirety) can predict long-term fibrosis progression in early-stage fibrotic liver disease, we analyzed the PLS in 43 patients with F0 or F1 fibrosis in the index liver biopsy (PLS validation set 1), among which 12 patients showed F-stage increase of two stages or more within 5 years (Table 1). The PLS profiles classified the patients into high-(n=14, 33%), intermediate-(n=12, 28%), or low-(n=17, 40%) risk group for fibrosis progression (FIG. 2A). The PLS prediction was significantly and independently associated with histological fibrosis progression in multivariable logistic regression adjusted for clinical confounding variables: ALT and platelet count (adjusted odds ratio [aOR], 10.86; 95% confidence interval [CI], 1.13-104.83), and with an area under the receiver operating characteristic curve (AUROC) of 0.81 (FIG. 2B and FIG. 2C, Tables 5 and 6). In this nested case-control series, 24 patients had HIV co-infection, a known fibrosis accelerator. HIV co-infection showed a trend of association with fibrosis progression, although insignificant likely due to the use of anti-retroviral therapies and limited sample size (univariable OR=3.20; 95% CI 0.73-14.12). The number of patients with active HCV infection has been declining with the widespread use of direct-acting antivirals (DAA), but this result demonstrates a proof of concept that hepatic transcriptome can predict long-term fibrosis progression in a major chronic liver disease etiology, sharing multiple molecular mechanisms of fibrogenesis with other etiologies.

TABLE 5
Factors associated with fibrosis progression in the validation
sets (Uni/multivariable logistic regression).
Table 5A: PLS validation set 1, FPS derivation set 1 (chronic hepatitis C, case-control, U.S.)

| | | Univariable | | | Multivariable | | |
|---|---|---|---|---|---|---|---|
| Variable | OR | 95% CI | P | OR | 95% CI | P | n (%) |
| PLS: intermediate- | 3.75 | (0.56-25.12) | .17 | 8.00 | (0.77-83.30) | .08 | 12 (28%) |
| risk (vs. low-risk) | | | | | | | |
| high-risk (vs. low- | 5.62 | (0.92-34.57) | .06 | 10.61 | (1.01-111.51) | .049 | 14 (33%) |
| risk) | | | | | | | |
| Male sex | 3.21 | (0.35-29.35) | .30 | | | | 35 (81%) |
| Age > 50 y | 0.57 | (0.10-3.21) | .53 | | | | 10 (23%) |
| Total bilirubin > 0.8 | 1.05 | (0.25-4.33) | .95 | | | | 14 (33%) |
| mg/dL | | | | | | | |
| Albumin < 4.0 g/dL | 1.05 | (0.25-4.33) | .95 | | | | 14 (33%) |
| AST > 40 IU/L | 7.12 | (1.31-38.76) | .02 | | | | 21 (49%) |
| ALT > 40 IU/L | 6.92 | (1.29-37.05) | .02 | 9.69 | (1.39-67.53) | .02 | 23 (53%) |
| Platelet count < | 3.42 | (0.86-13.67) | .08 | 7.89 | (1.19-52.17) | .03 | 16 (37%) |
| 200,000/mL | | | | | | | |
| HIV co-infection | 3.20 | (0.73-14.12) | .13 | | | | 24 (56%) |

Table 5B: PLS validation set 2, FPS derivation set 2 (transplantation, case-control, U.S.)

| | | Univariable | | | Multivariable | | |
|---|---|---|---|---|---|---|---|
| Variable | OR | 95% CI | P | OR | 95% CI | P | n (%) |
| PLS: intermediate-risk | 0.83 | (0.15-4.64) | .84 | 0.57 | (0.06-5.02) | .61 | 9 (24%) |
| (vs. low-risk) | | | | | | | |
| high-risk (vs. low-risk) | 20.00 | (2.05-195.01) | .009 | 26.50 | (1.97-355.61) | .01 | 13 (34%) |
| Male sex | 0.24 | (0.05-1.07) | .06 | | | | 25 66%) |
| Recipient age > 50 y | 3.67 | (0.95-14.09) | .06 | | | | 20 (53%) |
| Total bilirubin > 0.8 | 3.39 | (0.73-15.9) | .12 | | | | 11 (29%) |
| mg/dl | | | | | | | |
| Albumin < 3.5 g/dL | 1.41 | (0.21-9.62) | .73 | | | | 5 (13%) |
| AST > 40 IU/L | 5.36 | (1.24-23.21) | .03 | | | | 22 (58%) |
| ALT > 40 IU/L | 3.10 | (0.79-12.15) | .11 | | | | 20 (53%) |
| Platelet count < | 0.68 | (0.17-2.65) | .58 | | | | 25 (66%) |
| 200,000/mL | | | | | | | |
| Use of induction therapy | 11.67 | (2.44-55.83) | .002 | 18.18 | (2.44-135.65) | .005 | 18 (47%) |

Table 5C: FPS validation set 1 (NASH, case-control, Japan)

| | | Univariable | | | Multivariable | | |
|---|---|---|---|---|---|---|---|
| Variable | OR | 95% CI | P | OR | 95% CI | P | n (%) |
| FPS: intermediate-risk | 1.10 | (0.3-4.08) | .88 | 1.33 | (0.25-7.07) | .74 | 44 (56%) |
| (vs. low-risk) | | | | | | | |
| high-risk (vs. low-risk) | 1.36 | (0.28-6.68) | .70 | 10.93 | (1.11-107.78) | .04 | 15(19%) |
| Male sex | 1.29 | (0.44-3.77) | .65 | | | | 44 (56%) |
| Age > 60 y | 1.34 | (0.46-3.85) | .59 | | | | 39 (50%) |
| BMI > 30 kg/m² | 0.79 | (0.23-2.74) | .71 | | | | 20 (26%) |
| Diabetes | 0.43 | (0.13-1.46) | .18 | | | | 28 (36%) |
| NASH activity score | 1.13 | (0.81-1.58) | .48 | | | | |
| (NAS) | | | | | | | |
| Fibrosis stage | 0.10 | (0.03-0.3) | <.001 | 0.06 | (0.02-0.21) | <.001 | |
| Total bilirubin > 0.8 | 3.00 | (0.99-9.08) | .05 | | | | 36 (46%) |
| mg/dL | | | | | | | |
| Albumin < 4 g/dL | 3.47 | (0.21-58.45) | .39 | | | | 2(3%) |
| AST > 40 IU/L | 0.87 | (0.30-2.51) | .80 | | | | 41 (53%) |
| ALT > 40 IU/L | 0.57 | (0.10-3.41) | .54 | | | | 72 (92%) |
| Platelet count < | 0.83 | (0.26-2.66) | .75 | | | | 24 (31%) |
| 200,000/mL | | | | | | | |

Table 5D: FPS validation set 1 (NASH, case-control, Japan)

| | | Univariable | | | Multivariable | | |
|---|---|---|---|---|---|---|---|
| Variable | OR | 95% CI | P | OR | 95% CI | P | n (%) |
| FPS: intermediate- | 1.10 | (0.3-4.08) | .88 | 1.33 | (0.25-7.07) | .74 | 44 (56%) |
| risk (vs. low-risk) | | | | | | | |
| high-risk (vs. low- | 1.36 | (0.28-6.68) | .70 | 10.93 | (1.11-107.78) | .04 | 15(19% |
| risk) | | | | | | | |
| Male sex | 1.29 | (0.44-3.77) | .65 | | | | 44 (56%) |
| Age > 60 y | 1.34 | (0.46-3.85) | .59 | | | | 39 (50%) |
| BMI > 30 kg/m² | 0.79 | (0.23-2.74) | .71 | | | | 20 (26%) |
| Diabetes | 0.43 | (0.13-1.46) | .18 | | | | 28 (36%) |
| NASH activity score | 1.13 | (0.81-1.58) | .48 | | | | |
| (NAS) | | | | | | | |
| Fibrosis stage | 0.10 | (0.03-0.3) | <.001 | 0.06 | (0.02-0.21) | <.001 | |
| Total bilirubin > 0.8 | 3.00 | (0.99-9.08) | .05 | | | | 36 (46%) |
| mg/dL | | | | | | | |
| Albumin < 4 g/dL | 3.47 | (0.21-58.45) | .39 | | | | 2(3%) |
| AST > 40 IU/L | 0.87 | (0.30-2.51) | .80 | | | | 41 (53%) |
| ALT > 40 IU/L | 0.57 | (0.10-3.41) | .54 | | | | 72 (92%) |
| Platelet count < | 0.83 | (0.26-2.66) | .75 | | | | 24 (31%) |
| 200,000/mL | | | | | | | |

Table 5E: FPSec validation set (compensated cirrhosis with mixed etiologies, cohort, U.S.)*

| | | Univariable | | | Multivariable | | |
|---|---|---|---|---|---|---|---|
| Variable | OR | 95% CI | P | OR | 95% CI | P | n (%) |
| FPSec: high-risk (vs. | 3.94 | (1.59-9.78) | .003 | 3.00 | (1.16-7.79) | .02 | |
| low-risk) | | | | | | | |
| ALBI-FIB4 score: high- | 3.77 | (1.67-8.50) | .001 | 2.61 | (1.12-6.12) | .03 | |
| risk (vs. low-risk) | | | | | | | |

Table 5F:

| | | Univariable | | | Multivariable | | |
|---|---|---|---|---|---|---|---|
| Variable | OR | 95% CI | P | OR | 95% CI | P | n (%) |
| FPSec: high-risk | 3.94 | (1.59-9.78) | 0.003 | 2.66 | (1.04-6.75) | 0.04 | |
| (vs. low-risk) | | | | | | | |
| FIB-4 index > 3.25 | 5.28 | (1.99-12.02) | <.001 | 3.51 | (1.21-10.21) | 0.02 | |
| (vs. ≤ 3.25) | | | | | | | |
| ALBI grade 2 or 3 | 3.39 | (1.29-8.97) | 0.01 | 1.54 | (0.53-4.44) | 0.42 | |
| (vs. grade 1) | | | | | | | |
| FPS validation set 1 | | | | | | | |
| Age > 50 y | 0.45 | (0.14-1.46) | 0.183 | | | | 61 (78%) |

Table 5G: PLS validation set 1, FPS derivation set 1 (chronic hepatitis C, case-control, U.S.)

| | | Univariable | | | Multivariable | | |
|---|---|---|---|---|---|---|---|
| Variable | OR | 95% CI | p | OR | 95% CI | p | n (%) |
| PLS: intermediate- | 3.75 | (0.56-25.12) | 0.17 | 7.5 | (0.70-80.73) | 0.1 | 12 (28%) |
| risk (vs. low-risk) | | | | | | | |
| high-risk (vs. | 5.62 | (0.92-34.57) | 0.06 | 10.86 | (1.13-104.83) | 0.039 | 14 (33%) |
| low-risk) | | | | | | | |
| Male sex | 3.21 | (0.35-29.35) | 0.3 | | | | 35 (81%) |
| Age > 50 y | 0.57 | (0.10-3.21) | 0.53 | | | | 10 (23%) |
| Total bilirubin > | 1.05 | (0.25-4.33) | 0.95 | | | | 14 (33%) |
| 0.8 mg/dL | | | | | | | |
| Albumin < 4.0 g/dL | 1.05 | (0.25-4.33) | 0.95 | | | | 14 (33%) |
| AST > 40 IU/L | 7.12 | (1.31-38.76) | 0.023 | | | | 21 (49%) |
| ALT > 40 IU/L | 6.92 | (1.29-37.05) | 0.024 | 8.87 | (1.26-62.44) | 0.028 | 23 (53%) |
| Platelet count < | 3.42 | (0.86-13.67) | 0.08 | 7.33 | (1.12-48.17) | 0.038 | 16 (37%) |
| 200,000/mL | | | | | | | |
| HCV-RNA, | 1.75 | (0.40-7.70) | 0.46 | | | | 18 (42%) |
| >3,000 × 10³ IU/mL | | | | | | | |
| HIV co-infection | 3.20 | (0.73-14.12) | 0.125 | | | | 24 (56%) |
| CD4 < 400 /mL* | 0.25 | (0.04-1.44) | 0.12 | | | | 13 (30%) |

Table 5H: PLS validation set 2, FPS derivation set 2 (transplantation, case-control, U.S.)

| | | Univariable | | | Multivariable | | |
|---|---|---|---|---|---|---|---|
| Variable | OR | 95% CI | p | OR | 95% CI | p | n (%) |
| PLS: intermediate-risk | 0.83 | (0.15-4.64) | 0.84 | 0.57 | (0.06-5.02) | 0.612 | 9 (24%) |
| (vs. low-risk) | | | | | | | |
| high-risk (vs. | 20.00 | (2.05-195.01) | 0.009 | 26.50 | (1.97-355.61) | 0.013 | 13 (34%) |
| low-risk) | | | | | | | |
| Male sex | 0.24 | (0.05-1.07) | 0.06 | | | | 25 66%) |
| Recipient age > 50 y | 3.67 | (0.95-14.09) | 0.06 | | | | 20 (53%) |
| BMI > 30 kg/m² | 1.22 | (0.32-4.66) | 0.77 | | | | 14 (37%) |
| Diabetes - pre- | 0.40 | (0.08-2.00) | 0.26 | | | | 8 (21%) |
| transplant | | | | | | | |
| Total bilirubin > 0.8 | 3.39 | (0.73-15.9) | 0.12 | | | | 11 (29%) |
| mg/dL | | | | | | | |
| Albumin < 3.5 g/dL | 1.41 | (0.21-9.62) | 0.73 | | | | 5 (13%) |
| AST > 40 IU/L | 5.36 | (1.24-23.21) | 0.03 | | | | 22 (58%) |
| ALT > 40 IU/L | 3.10 | (0.79-12.15) | 0.11 | | | | 20 (53%) |
| Platelet count < | 0.68 | (0.17-2.65) | 0.58 | | | | 25 (66%) |
| 200,000/mL | | | | | | | |
| HCV-RNA > | 0.57 | (0.12-2.55) | 0.45 | | | | 9 (24%) |
| 1,000 × 10³ IU/mL | | | | | | | |
| Cytomegalovirus | 2.67 | (0.25-28.28) | 0.42 | | | | 4 (11%) |
| infection | | | | | | | |
| Use of induction therapy | 11.67 | (2.44-55.83) | 0.002 | 18.18 | (2.44-135.65) | 0.005 | 18 (47%) |
| Use of tacrolimus (vs. CsA) | 1.87 | (0.39-8.93) | 0.43 | | | | 23 (61%) |

Table 5I: FPS validation set 1 (NASH, case-control, Japan)

| | | Univariable | | | Multivariable | | |
|---|---|---|---|---|---|---|---|
| Variable | OR | 95% CI | p | OR | 95% CI | p | n (%) |
| FPS: | 1.10 | (0.3-4.08) | 0.883 | 1.33 | (0.25-7.07) | 0.736 | 44 (56%) |
| intermediate-risk | | | | | | | |
| (vs. low-risk) | | | | | | | |
| high-risk (vs. | 1.36 | (0.28-6.68) | 0.702 | 10.93 | (1.11-107.78) | 0.041 | 15(19%) |
| low-risk) | | | | | | | |
| Male sex | 1.29 | (0.44-3.77) | 0.647 | | | | 44 (56%) |
| Age > 60 y | 1.34 | (0.46-3.85) | 0.592 | | | | 39 (50%) |
| BMI > 30 kg/m² | 0.79 | (0.23-2.74) | 0.705 | | | | 20 (26%) |
| Diabetes | 0.43 | (0.13-1.46) | 0.175 | | | | 28 (36%) |
| NASH activity | 1.13 | (0.81-1.58) | 0.475 | | | | |
| score (NAS) | | | | | | | |
| Fibrosis stage | 0.10 | (0.03-0.3) | <.001 | 0.06 | (0.02-0.21) | <0.001 | |
| Platelet count < | 0.83 | (0.26-2.66) | 0.754 | | | | 24 (31%) |
| 200,000/mL | | | | | | | |
| Total bilirubin > | 3.00 | (0.99-9.08) | 0.052 | | | | 36 (46%) |
| 0.8 mg/dL | | | | | | | |
| Albumin < 4 g/dL | 3.47 | (0.21-58.45) | 0.388 | | | | 2(3%) |
| AST > 40 IU/L | 0.87 | (0.30-2.51) | 0.804 | | | | 41 (53%) |
| ALT > 40 IU/L | 0.57 | (0.10-3.41) | 0.539 | | | | 72 (92%) |

OR: odds ratio, AST: aspartate aminotransferase, ALT: alanine aminotransferase, BMI: body mass index.
*Hazard ratio (HR) from Cox regression is shown.
*In HIV-co-infected patients.
OR: odds ratio, HR: hazard ratio, BMI: body mass index, AST: aspartate aminotransferase, ALT: alanine aminotransferase, azathioprine, ALP: alkaline phosphatase, CsA: cyclosporine A.

### Table 6: Performance of the PLS/FPS and Clinical Prognostic Variables.

TABLE 6
Performance of the PLS/FPS and clinical prognostic variables.
Table 6A: PLS validation

| | | | | | | |
|---|---|---|---|---|---|---|
| | AUROC | | Residual | | LR test (chi- | Sensitivity/ |
| Model | (95% CI) | R² | deviance | AIC | square; P) | Specificity |

PLS validation set 1

| | | | | | | |
|---|---|---|---|---|---|---|
| PLS only | 0.70 | 0.09 | 47.03 | 51.04 | — | 83%, 68% |
| | (0.68, 0.83) | | | | | |
| Clinical | 0.70 | 0.13 | 44.86 | 48.86 | — | 83%, 55% |
| variable only | (0.52-0.89) | | | | | |

PLS validation set 2

| | | | | | | |
|---|---|---|---|---|---|---|
| Clinical | 0.81 | 0.25 | 38.16 | 44.15 | 6.7; .009 | 75%, 90% |
| variable + PLS | (0.62-0.99) | | | | | |
| PLS only | 0.80 | 0.26 | 41.6 | 45.60 | — | 71%, 94% |
| | (0.66-0.95) | | | | | |
| Clinical | 0.77 | 0.29 | 40.65 | 44.65 | — | 71%, 82% |
| variable only | (0.63-0.91) | | | | | |
| Clinical | 0.87 | 0.42 | 33.34 | 39.34 | 7.3; .006 | 81%, 88% |
| variable + PLS | (0.75-0.99) | | | | | |

Table 6B: FPS derivation set

| | | | | | | |
|---|---|---|---|---|---|---|
| | AUROC | | Residual | | LR test (chi- | Sensitivity/ |
| Model | (95% CI) | R² | deviance | AIC | square; P) | Specificity |

FPS derivation set 1

| | | | | | | |
|---|---|---|---|---|---|---|
| FPS only | 0.67 | 0.07 | 47.83 | 51.83 | — | 58%, 77% |
| | (0.48-0.86) | | | | | |
| Clinical | 0.72 | 0.13 | 44.86 | 48.86 | — | 83%, 55% |
| variable only | (0.518-0.89) | | | | | |
| Clinical | 0.83 | 0.23 | 38.22 | 44.22 | 6.64; .009 | 83%, 81% |
| variable + FPS | (0.66-1.00) | | | | | |

FPS derivation set 2

| | | | | | | |
|---|---|---|---|---|---|---|
| FPS only | 0.79 | 0.27 | 41.19 | 45.19 | — | 76%, 77% |
| | (0.64-0.94) | | | | | |
| Clinical | 0.77 | 0.29 | 40.65 | 44.65 | — | 71%, 82% |
| variable only | (0.63-0.91) | | | | | |
| Clinical | 0.86 | 0.4 | 34.61 | 40.61 | 6.05; .014 | 86%, 82% |
| variable + FPS | (0.73-0.98) | | | | | |

Table 6C: FPS validation set

| | | | | | | |
|---|---|---|---|---|---|---|
| | AUROC | | Residual | | LR test (chi- | Sensitivity/ |
| Model | (95% CI) | R² | deviance | AIC | square; P) | Specificity |

FPS validation set 1

| | | | | | | |
|---|---|---|---|---|---|---|
| FPS only | 0.52 | 0.00 | 84.19 | 88.19 | — | 94%, 20% |
| | (0.36-0.67) | | | | | |
| Clinical | 0.83 | 0.29 | 58.75 | 67.74 | — | 67%, 92% |
| variable only | (0.73-0.94) | | | | | |
| Clinical | 0.86 | 0.32 | 55.78 | 61.78 | 2.96; .085 | 83%, 83% |
| variable + FPS | (0.76-0.96) | | | | | |

PLS, Prognostic Liver Signature; AUROC, area under the receiver operating characteristic; LR, likelihood ratio.
FPS, Fibrosis Progression Signature; AUROC, area under the receiver operating characteristic; LR, likelihood ratio.

### Example 2: PLS is Associated with 5-Year Fibrosis Progression after Liver Transplantation

The association of PLS with fibrosis progression was further validated in another clinical scenario, liver transplantation. Fibrosis progression after transplantation due to recurrent HCV infection has been the major problem that limits patient survival. Sustained virologic response (SVR) to anti-HCV therapies improves surrogate indicators of fibrosis such as liver stiffness in short-term, which is followed by gradual regression of histological fibrosis. However, SVR rate with DAA post transplantation can be as low as 50% and adverse event rates can be as high as 75% when progressed to decompensated liver disease. Therefore, prediction of fibrosis progression will remain relevant in a subset of post-transplant patients with HCV infection. To evaluate PLS for its capability to estimate risk of future fibrosis progression, we analyzed liver biopsy tissues obtained one year after receiving liver transplantation in 38 HCV cirrhosis patients with F0 or F1 fibrosis, including 21 fibrosis progressors and 17 non-progressors (PLS validation set 2). The PLS profiles classified the patients into high-(n=13, 34%), intermediate-(n=9, 24%), or low-(n=16, 42%) risk groups (FIG. 2A). The presence of a high-risk PLS was significantly associated with histological fibrosis progression in multivariable logistic regression adjusted for clinical confounding variables (aOR, 26.50; 95% CI, 1.97-355.61) and with an AUROC of 0.87 (FIG. 2B and FIG. 2C, Tables 5 and 6). In summary, the association of PLS with histological fibrosis progression was successfully validated in two clinical scenarios, i.e., chronic hepatitis and post transplantation, in patients with no to minimal fibrosis.

### Example 3: FPS Shared Between Viral and Metabolic Liver Disease Etiologies was Defined

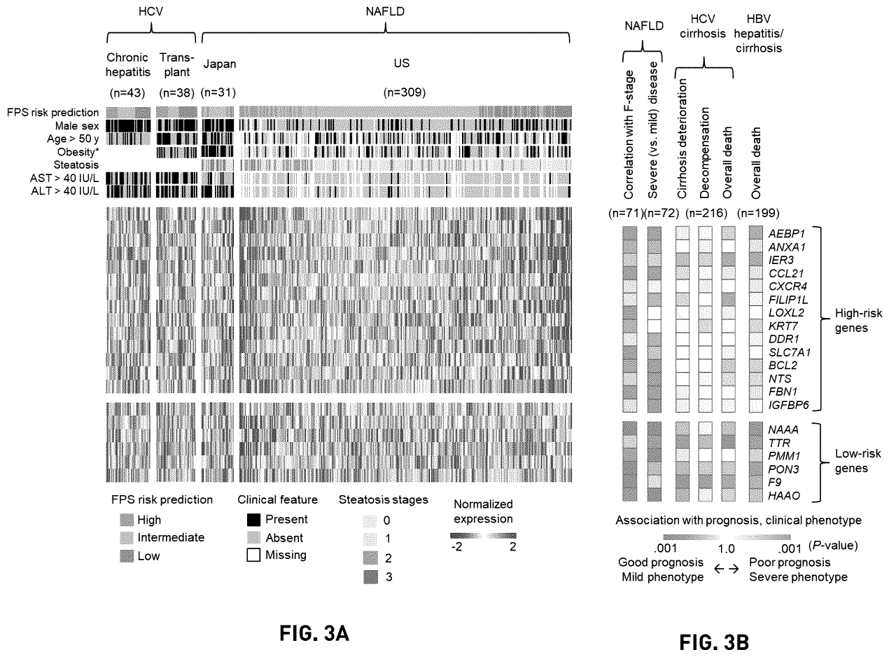

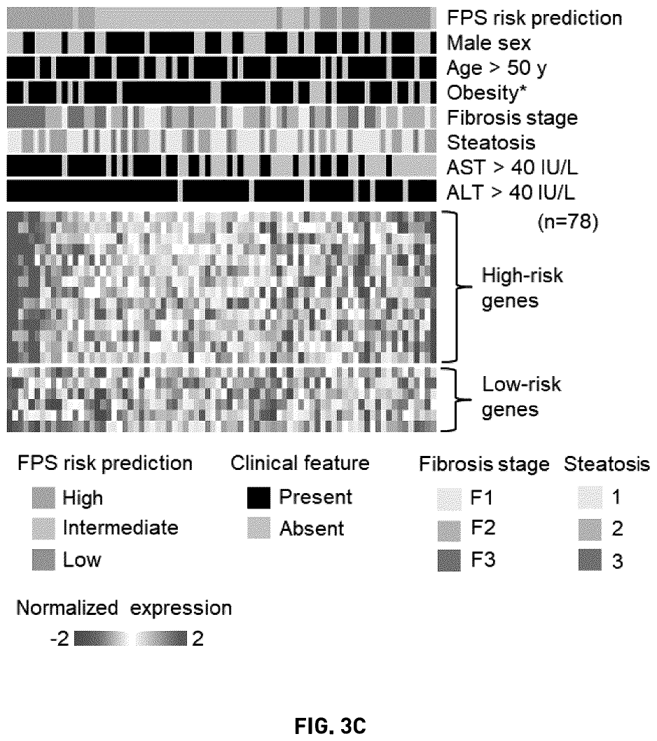

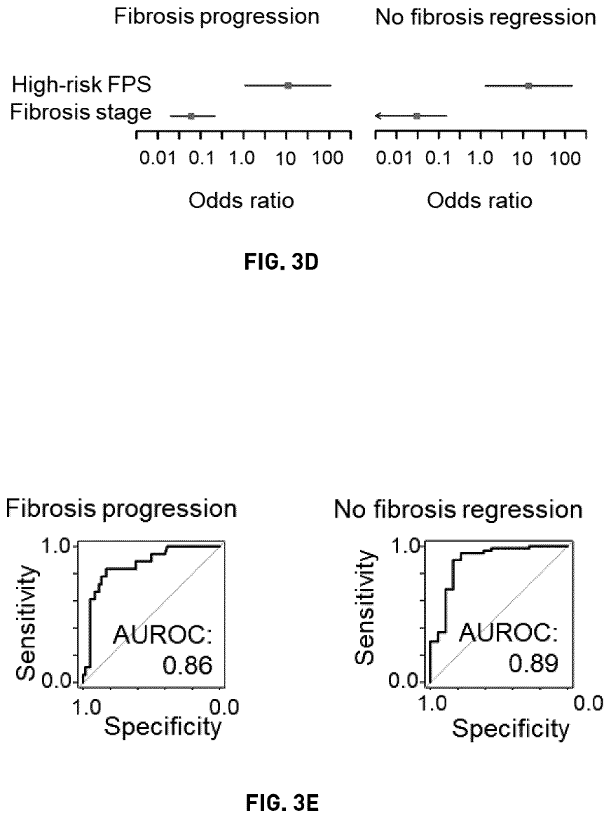

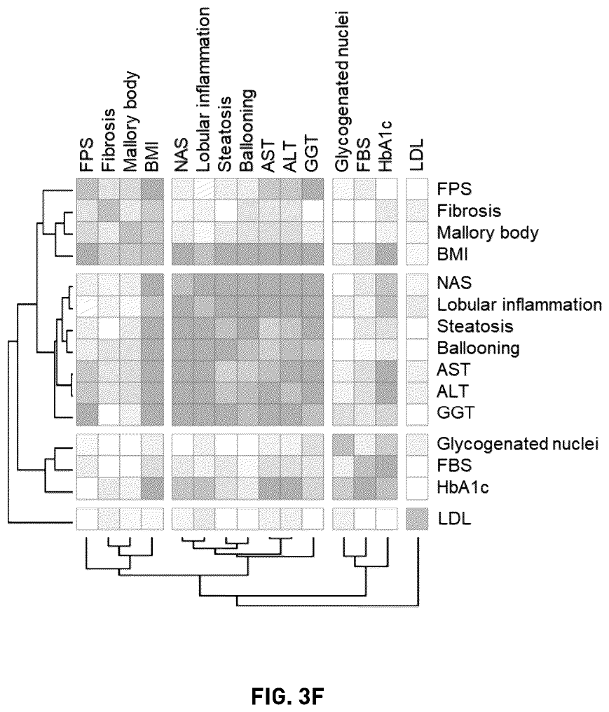

With the encouraging validation of the PLS in patients with early-stage liver disease, indicating that the hepatic transcriptome informs future progression of fibrotic liver disease, we next sought to define a molecular signature more specifically associated with fibrosis progression. Progressive fibrosis is a common feature shared among viral and metabolic liver disease etiologies. Consistent with the notion, our PLS predicts adverse outcomes in patients with advanced liver diseases caused by viral and metabolic etiologies. To define a transcriptomic signature associated with long-term fibrosis progression in an etiology-agnostic manner, we integrated the FPS derivation sets 1 to 4, representing major viral (HCV) and metabolic (NAFLD) etiologies (421 patients in total) (FIG. 1, Table 1), for association with time to fibrosis progression and transcriptomic co-expression shared between HCV and NAFLD (FIG. 8A, FIG. 8B, see Methods and Materials above). A 20-gene FPS, consisting of 14 high- and 6 low-risk genes were identified (FIG. 3A, Table 7). Some of the FPS member genes, e.g., CCL21 and LOXL2, were individually implicated in liver fibrogenesis in chemical and physiological liver fibrosis models as well as fibrotic liver disease patients, supporting the validity of our approach to identify molecular drivers of liver fibrosis relevant to broad biological and clinical contexts.

TABLE 7
Fibrosis Progression Signature (FPS).

| Gene | Gene | | Fibrosis risk | Fisher's |
|---|---|---|---|---|
| symbol | ID | Gene name | association | ICS* |
| AEBP1 | 165 | AE Binding Protein 1 | High-risk | 3.67 |
| ANXA1 | 301 | Annexin A1 | High-risk | 2.66 |
| IER3 | 8870 | Immediate Early Response 3 | High-risk | 2.58 |
| CCL21 | 6366 | C-C Motif Chemokine | High-risk | 2.35 |
| | | Ligand 21 | | |
| CXCR4 | 7852 | C-X-C Motif Chemokine | High-risk | 2.33 |
| | | Receptor 4 | | |
| FILIP1L | 11259 | Filamin A Interacting Protein | High-risk | 2.06 |
| | | 1 Like | | |
| LOXL2 | 4017 | Lysyl Oxidase Like 2 | High-risk | 2.03 |
| KRT7 | 3855 | Keratin 7 | High-risk | 1.89 |
| DDR1 | 780 | Discoidin Domain Receptor | High-risk | 1.56 |
| | | Tyrosine Kinase 1 | | |
| SLC7A1 | 6541 | Solute Carrier Family 7 | High-risk | 1.54 |
| | | Member 1 | | |
| BCL2 | 596 | BCL2 Apoptosis Regulator | High-risk | 1.48 |
| NTS | 4922 | Neurotensin | High-risk | 1.46 |
| FBN1 | 2200 | Fibrillin 1 | High-risk | 1.43 |
| IGFBP6 | 3489 | Insulin Like Growth Factor | High-risk | 1.36 |
| | | Binding Protein 6 | | |
| ASAHL | 27163 | N-Acylethanolamine Acid | Low-risk | −1.37 |
| (NAAA) | | Amidase | | |
| TTR | 7276 | Transthyretin | Low-risk | −1.76 |
| PMM1 | 5372 | Phosphomannomutase 1 | Low-risk | −1.78 |
| PON3 | 5446 | Paraoxonase 3 | Low-risk | −1.80 |
| F9 | 2158 | Coagulation Factor IX | Low-risk | −2.17 |
| HAAO | 23498 | 3-Hydroxyanthranilate 3,4- | Low-risk | −2.59 |
| | | Dioxygenase | | |

*Fisher's inverse chi-square statistic synthesizing Cox score p-values in the HCV cohorts with longitudinal follow-up.

FPS-based prognostic prediction is correlated with PLS-based prediction, while it is not completely overlapping, particularly in patients with a metabolic etiology (concordance rates are 72%, 82%, 74%, and 62% in the FPS derivation sets 1, 2, 3, and 4, respectively) (FIG. 8C). The proportion of high-risk prediction is smaller for FPS (14%) compared to PLS (24%) among the patients in the four FPS derivation sets, suggesting that FPS identifies a subset of high-risk PLS patients with elevated risk of fibrosis progression. In the FPS derivation set 1, 13 patients were predicted as having a high-risk of disease progression by the PLS, among which 5 patients (38%) showed 5-year fibrosis progression. Among 11 patients predicted as having a high-risk of disease progression by the FPS, 5 patients (45%) showed 5-year fibrosis progression (FIG. 8D). In the FPS derivation set 2, 13 patients were predicted as having a high-risk of disease progression by the PLS, among which 12 patients (92%) showed 5-year fibrosis progression. Among 11 patients predicted as having a high-risk of disease progression by the FPS, 10 patients (91%) showed 5-year fibrosis progression (FIG. 8E). Furthermore, in multiple independent patient cohorts representing diverse liver disease etiologies, namely HBV, HCV, ALD, and NAFLD (Table 8), FPS genes were associated with fibrotic liver disease severity and adverse outcomes (FIG. 3B). These results collectively warranted further independent validation of FPS for fibrosis progression.

TABLE 8
Transcriptome data sets of clinical chronic liver disease patients and
experimental animal models from public database.
TABLE 8A:

| First | | | | Age, | |
|---|---|---|---|---|---|
| author, | | | Male, | median | Major |
| year | n | Species | n (%) | (IQR) | etiology |
| Ahrens, | 71 | Human | 15 (21%) | 45 | NAFLD |
| 2013 | | | | (38-51) | |
| Moylan, | 72 | Human | 25 (35%) | 50-52 | NAFLD |
| 2014 | | | | | |
| Hoshida, | 216 | Human | 116 (54%) | 59 | HCV |
| 2013 | | | | (54-64) | |
| Roessler, | 199 | Human | 175 (88%) | 50 | HBV |
| 2010 | | | | (44-58) | |
| Rama- | 3 | Human | 2 (67%) | n.a. | Alcohol, |
| chandran, | | | | | NAFLD |
| 2019 | | | | | |
| Stephen, | 25 | Mouse | n.a. | n.a. | n.a. |
| 2015 | | | | | |
| McMullen, | 4 | Human | n.a. | n.a. | n.a. |
| 2014 | | | | | |

TABLE 8B:

| | Advanced | | |
|---|---|---|---|
| First | fibrosis or | | |
| author, | cirrhosis, | Race/ | |
| year | n (%) | ethnicity | Correlated phenotype |
| Ahrens, | 4 (6%) | White | Histological fibrosis |
| 2013 | | | stage |
| Moylan, | 32 | White; | Advanced (n = 32) vs. |
| 2014 | (44%) | Black; | mild (n = 40) NAFLD |
| | | Asian; | |
| | | Hawaiian | |
| | | Pacific | |
| | | islander | |
| Hoshida, | 216 | White | Time to cirrhosis |
| 2013 | (100%) | | progression, |
| | | | decompensation, death* |
| Roessler, | 185 (93%) | Asian | Time to overall death |
| 2010 | | | |
| Rama- | 3 | n.a. | Clinical diagnosis of |
| chandran, | (100%) | | cirrhosis |
| 2019 | | | |
| Stephen, | n.a. | n.a. | PPAR agonist treatment |
| 2015 | | | |
| McMullen, | n.a. | n.a. | GW7647 treatment |
| 2014 | | | |

TABLE 8C:

| First | | | |
|---|---|---|---|
| author, | | | |
| year | GEO ID | PMID | Reference (PMID) |
| Ahrens, | GSE48452 | 23931760 | Cell Metab 18; 296, |
| 2013 | | | 2013 (23931760) |
| Moylan, | GSE49541 | 23913408 | Hepatol 59; 471, |
| 2014 | | | 2014 (23913408) |
| Hoshida, | GSE15654 | 23333348 | Gastro 144; 1024, |
| 2013 | | | 2013 (23333348) |
| Roessler, | GSE14520 | 21159642 | Cancer Res 70; 10202, 2010 |
| 2010 | | | (21159642) |
| Rama- | GSE136103 | 31597160 | Nature 575; 7783, |
| chandran, | | | 2019 (31597160) |
| 2019 | | | |
| Stephen, | GSE27948 | Unpublished | Unpublished |
| 2015 | | | |
| McMullen, | GSE53399 | 24269660 | Chem Biol Interact 209: |
| 2014 | | | 14, 2014 (24269660) |

*Cirrhosis progression is defined as progression of Chilld-Pugh class A to class B or C; decompensation is defined as development of variceal bleeding, encephalopathy, ascites, and/or spontaneous bacterial peritonitis; death is defined as overall death.

### Example 4: FPS Predicts Fibrosis Progression in NAFLD

To validate FPS for its association with fibrosis progression, we profiled liver biopsy tissues from an independent cohort of 78 NAFLD patients (FPS validation set 1). FPS classified the patients into high-(n=15; 19%), intermediate-(n=44; 56%), and low-(n=19; 24%) risk groups (FIG. 3C). Changes in histological fibrosis were assessed in follow-up biopsy performed with a median interval of 2.4 years (IQR: 2.2-3.0 years). A high-risk FPS at baseline was significantly associated with the primary endpoint of this study, i.e., progression of fibrosis stage by one or more (aOR, 10.93; 95% CI, 1.11-107.78; P=0.04), as well as no fibrosis regression (aOR, 13.66; 95% CI, 1.28-145.29) (FIG. 3D, Table 5). A high-risk PLS showed association with fibrosis progression (OR, 3.67; 95% CI: 0.57-23.47) and no fibrosis regression (adjusted OR, 6.83; 95% CI, 1.04-44.87) to lesser extent compared to FPS, suggesting superiority of FPS in estimating risk of fibrosis progression. AUROCs of high-risk FPS are >0.86 for fibrosis progression and no fibrosis regression, supporting its predictive performance (FIG. 3E). The FPS risk predictions changed in the follow-up biopsy along with the F-stage from the baseline, while the predictions were generally correlated (FIG. 8F).

Assessments on whether change in FPS status over the course of clinical follow-up is associated with changes of histological and/or clinical features were conducted. It was observed that the FPS change was closely correlated with time-adjusted change in histological fibrosis stage along with Mallory body (FIG. 3F). The second closest features include hepatic steatosis, hepatocyte ballooning, and histological/biochemical inflammation as well as BMI. It was also observed weak correlations with glucose-metabolism-related features (HbAlc, fasting blood glucose, and glycogenated nuclei) and LDL cholesterol. This result suggests that FPS reflects dynamic change in fibrotic, steatotic, and inflammatory histological features in NAFLD liver.

There is a clinical need for biomarkers to detect presence of substantial fibrosis for indication of possible medical intervention. In the FPS validation set 1, there was a trend of association for high-risk FPS with ≥F2 fibrosis, but not statistically significant (FIG. 8G).

### Example 5: BCL2 is a Clinically-Relevant Pharmacological Anti-Fibrosis Target Encoded in the FPS

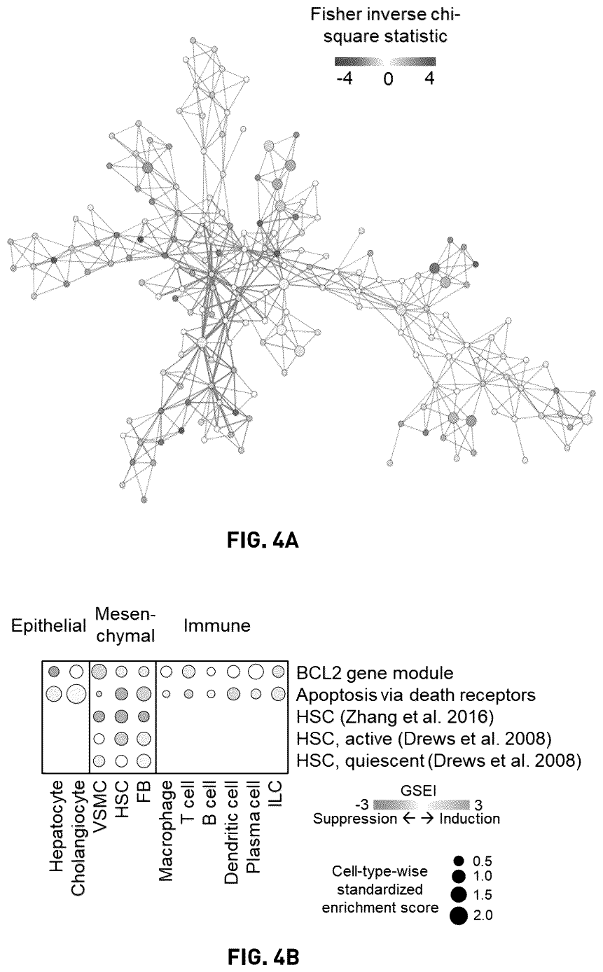

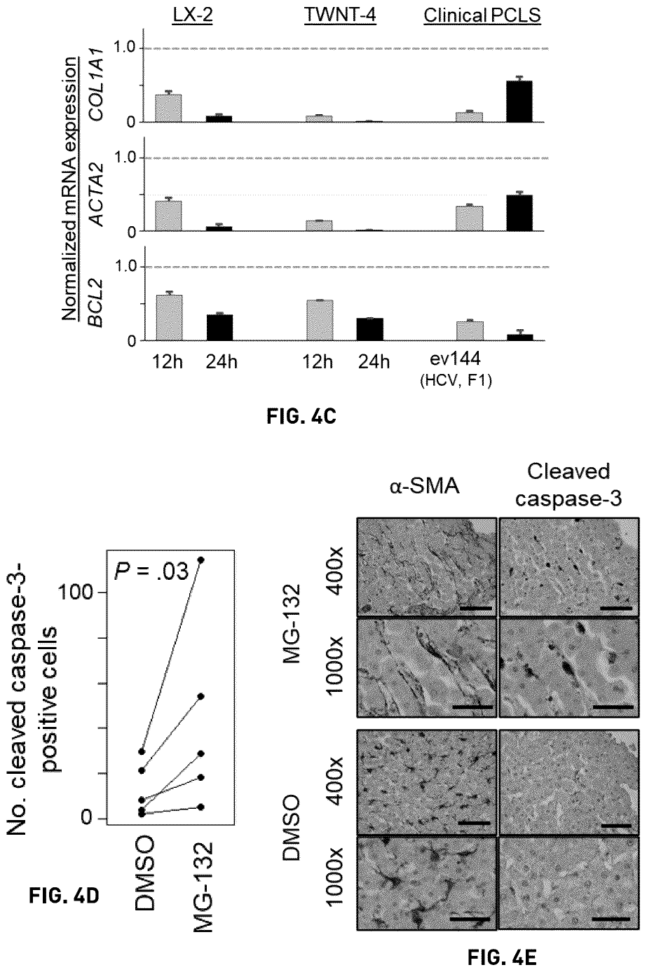

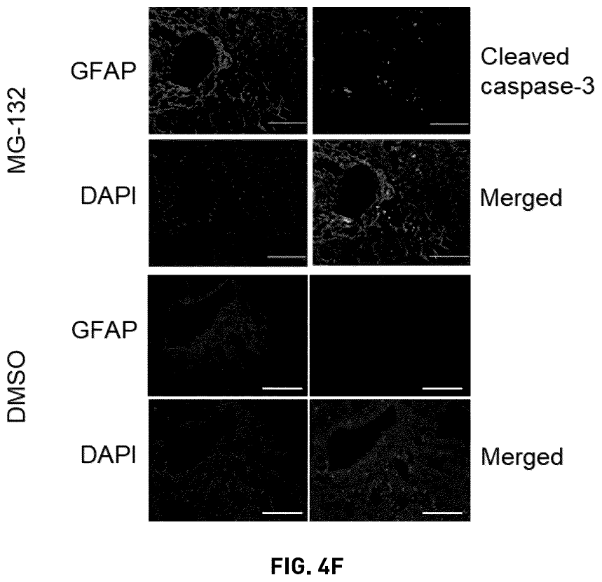

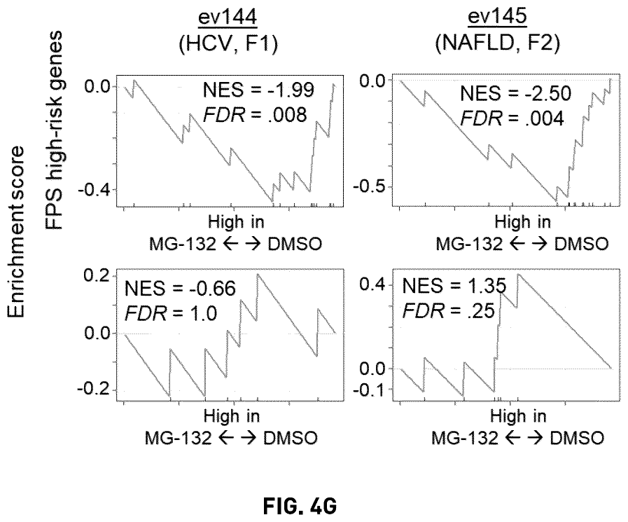

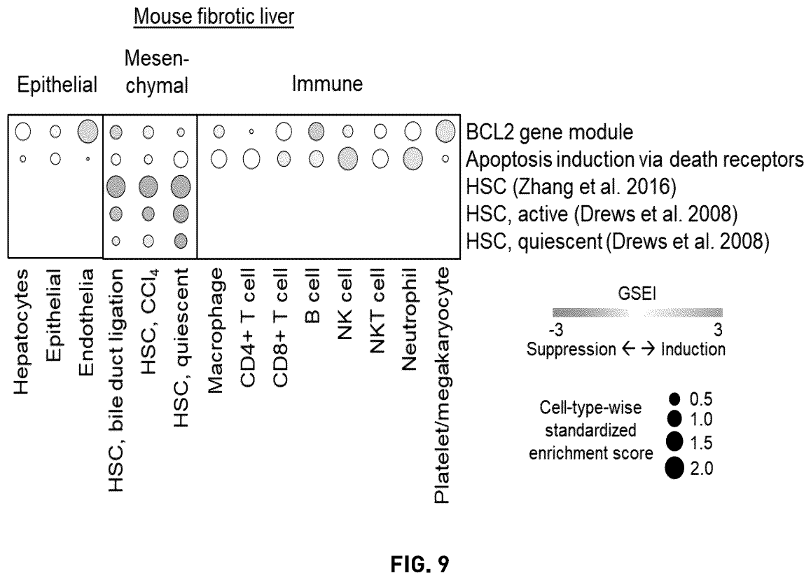

With the validated association of FPS with fibrosis progression, we next sought to determine whether FPS member genes/proteins represent clues to anti-fibrotic targets, for which FPS serves as a companion biomarker. Firstly, co-expression gene networks was developed by integrating the FPS derivation sets 1 to 4 using the MEGENA algorithm and inferred which FPS member genes likely have regulatory role (as fibrosis risk driver genes) to shape the fibrogenesis-promoting hepatic transcriptome (FIG. 4A, see Materials and Methods, above). One of the driver genes, B-cell lymphoma 2 (BCL2), was reported to be over-expressed in human cirrhotic livers and its genetic knockdown with siRNA sensitized myofibroblasts to apoptosis in cell culture experiment. An induction of co-regulated genes with BCL2 and suppression of apoptosis pathway in myofibroblasts was observed in single-cell transcriptome profiles of human cirrhotic livers (FIG. 4B). The same trends were observed in mouse liver cell transcriptome profiles (FIG. 9).

To test whether BCL2 activation in myofibroblasts can be pharmacologically inhibited as a clinically available anti-fibrotic strategy, computational screening was done in a collection of transcriptomic perturbations by 19,811 bioactive agents (CMap database). Several compounds were identified that mimic global transcriptomic modulation by BCL2 gene knockdown, including MG-132 and bortezomib, which are known as proteasome inhibitors (Table 9). These compounds indeed reduced myofibroblast activation and inhibit biliary fibrosis induced by bile duct ligation in mice. The effect of MG-132 was validated in human myofibroblast cell lines, LX2 and TWNT4, for suppression of type I collagen (COL1A1) and α-smooth muscle actin (ACTA2), hallmarks of hepatic fibrogenesis along with BCL2 (FIG. 4C). Furthermore, it was confirmed that MG-132 reduced expression of the genes in organotypic ex vivo culture of human precision-cut liver slice (PCLS) from two patients with fibrosis caused by HCV (F1) and NAFLD (F2) (FIG. 4C), accompanied with apoptosis induction shown by increased cleaved-caspase-3-positive cells (FIG. 4D) along sinusoidal area where α-smooth muscle actin (α-SMA) is present (FIG. 4E). Cleaved caspase-3 was co-localized with a stellate cell marker, glial fibrillary acidic protein (GFAP) in the MG-132-treated PCLS tissue compared to DMSO-treated tissue (FIG. 4F). Furthermore, in the clinical fibrotic tissues, ex vivo treatment with MG-132 significantly suppressed the high-risk FPS genes (false discovery rate [FDR]≤0.008), supporting the role of BCL2 in regulating high-risk FPS genes in human fibrotic liver (FIG. 4G). In summary, these data collectively suggest that FPS provides clues to clinically relevant anti-fibrotic targets, and serves as a readout to monitor effect of candidate anti-fibrotic agents in patient-derived fibrotic liver tissues.

Table 9A: Compounds that generate transcriptional modulation similar to BCL2 knockdown.

| cmap ID | Compound | Connectivity score |
|---|---|---|
| BRD-K60230970 | MG-132 | 98.18 |
| BRD-K88510285 | bortezomib | 96.69 |
| BRD-K13169950 | NSC-3852 | 96.35 |
| BRD-K78659596 | MLN-2238 | 96.17 |
| BRD-K37392901 | NSC-632839 | 95.98 |
| BRD-K09854848 | MD-II-008-P | 95.46 |

Table 9B: Compound digest: MG-132 Consensus Knockdown Connections

| | cmap_iname | score_best4 | score_best6 | ncell | nsig | |
|---|---|---|---|---|---|---|
| cmap_id | cmap_id | cmap_iname | score_best4 | score_best6 | ncell | nsig |
| 1 | CGS001-5682 | PSMA1 | 99.8793 | 97.937 | 7 | 7 |
| 2 | CGS001-5684 | PSMA3 | 99.7165 | 93.9296 | 9 | 9 |
| 3 | CGS001-5690 | PSMB2 | 99.5973 | 98.9624 | 8 | 10 |
| 4 | CGS001-5707 | PSMD1 | 99.583 | 95.9229 | 7 | 9 |
| 5 | CGS001-7316 | UBC | 99.0354 | 88.088 | 9 | 9 |
| 6 | CGS001-27243 | CHMP2A | 99.0279 | 91.1093 | 8 | 8 |
| 7 | CGS001-3309 | HSPA5 | 98.8802 | 98.1357 | 6 | 8 |
| 8 | CGS001-8892 | EIF2B2 | 98.7217 | 89.6259 | 8 | 10 |
| 9 | CGS001-26993 | AKAP8L | 98.3807 | 89.8767 | 9 | 11 |
| 10 | CGS001-350 | APOH | 98.2393 | 95.8833 | 9 | 9 |
| 11 | CGS001-7494 | XBP1 | 98.0229 | 96.0323 | 9 | 11 |
| 12 | CGS001-10972 | TMED10 | 98.0098 | 96.755 | 9 | 11 |
| 13 | CGS001-6428 | SRSF3 | 97.9266 | 95.5059 | 9 | 11 |
| 14 | CGS001-11331 | PHB2 | 97.769 | 95.1147 | 6 | 8 |
| 15 | CGS001-7415 | VCP | 97.7431 | 95.4454 | 7 | 7 |
| 16 | CGS001-10284 | SAP18 | 97.6604 | 80.0669 | 9 | 11 |
| 17 | CGS001-1314 | COPA | 97.3617 | 89.294 | 8 | 8 |
| 18 | CGS001-29978 | UBQLN2 | 97.3612 | 89.0856 | 9 | 11 |
| 19 | CGS001-11245 | GPR176 | 97.0331 | 93.1663 | 8 | 8 |
| 20 | CGS001-5373 | PMM2 | 97.0001 | 93.648 | 9 | 11 |
| 21 | CGS001-51160 | VPS28 | 96.9231 | 91.1237 | 9 | 11 |
| 22 | CGS001-5693 | PSMB5 | 96.8504 | 88.2169 | 7 | 7 |
| 23 | CGS001-9276 | COPB2 | 96.7666 | 88.7514 | 9 | 10 |
| 24 | CGS001-1454 | CSNK1E | 96.6104 | 91.0289 | 9 | 11 |
| 25 | CGS001-9341 | VAMP3 | 96.6045 | 89.6434 | 8 | 8 |
| 26 | CGS001-146540 | ZNF785 | 96.6034 | 93.795 | 9 | 9 |
| 27 | CGS001-79724 | ZNF768 | 96.5475 | 94.1015 | 9 | 10 |
| 28 | CGS001-5709 | PSMD3 | 96.52 | 94.3762 | 9 | 9 |
| 29 | CGS001-11284 | PNKP | 96.4634 | 94.1407 | 9 | 11 |
| 30 | CGS001-5763 | PTMS | 96.2689 | 93.1176 | 8 | 8 |
| 31 | CGS001-22818 | COPZ1 | 96.1344 | 93.0345 | 8 | 9 |
| 32 | CGS001-5708 | PSMD2 | 96.1001 | 94.0075 | 9 | 9 |
| 33 | CGS001-7272 | TTK | 96.0562 | 93.4388 | 8 | 10 |
| 34 | CGS001-22926 | ATF6 | 95.8852 | 88.2249 | 8 | 8 |
| 35 | CGS001-5608 | MAP2K6 | 95.8581 | 82.6887 | 8 | 8 |
| 36 | CGS001-51586 | MED15 | 95.7916 | 80.3359 | 9 | 9 |
| 37 | CGS001-56647 | BCCIP | 95.7572 | 87.8099 | 9 | 9 |
| 38 | CGS001-6723 | SRM | 95.6859 | 92.076 | 8 | 8 |
| 39 | CGS001-8894 | EIF2S2 | 95.6368 | 86.4113 | 7 | 9 |
| 40 | CGS001-2230 | FDX1 | 95.4533 | 93.0344 | 9 | 11 |
| 41 | CGS001-10856 | RUVBL2 | 95.301 | 87.3122 | 9 | 9 |
| 42 | CGS001-7314 | UBB | 95.2967 | 92.8972 | 8 | 8 |
| 43 | CGS001-2896 | GRN | 95.1931 | 88.9615 | 9 | 11 |
| 44 | CGS001-9318 | COPS2 | 95.0913 | 90.5768 | 8 | 8 |

Table 9C: Overexpression Connections

| | cmap_id | cmap_iname | score_best4 | score_best6 | ncell | nsig |
|---|---|---|---|---|---|---|
| 1 | ccsbBroad304_00833 | IFNG | 98.2871 | 91.9149 | 9 | 9 |
| 2 | ccsbBroad304_02048 | BCL10 | 97.1459 | 94.6832 | 8 | 8 |
| 3 | ccsbBroad304_05098 | MAGEB6 | 96.7305 | 96.2211 | 9 | 9 |
| 4 | ccsbBroad304_07117 | UGCG | 95.5545 | 83.2468 | 7 | 7 |
| 5 | ccsbBroad304_01090 | NFE2L2 | 94.9535 | 72.2845 | 7 | 10 |
| 6 | ccsbBroad304_01388 | RELB | 91.9166 | 88.5019 | 7 | 7 |
| 7 | ccsbBroad304_01710 | TRAF2 | 91.5627 | 77.2075 | 7 | 7 |
| 8 | ccsbBroad304_09396 | TIRAP | 90.7755 | 83.7322 | 7 | 7 |
| 9 | ccsbBroad304_00602 | SLC37A4 | 90.6113 | 83.0623 | 7 | 7 |
| 10 | ccsbBroad304_00832 | IFNB1 | 90.475 | 83.6629 | 9 | 9 |
| 11 | ccsbBroad304_00763 | HNF4A | 89.7731 | 69.9862 | 7 | 12 |
| 12 | ccsbBroad304_06542 | LTBR | 88.9201 | 87.383 | 9 | 9 |
| 13 | ccsbBroad304_00259 | CD40 | 88.3406 | 83.9532 | 8 | 8 |
| 14 | ccsbBroad304_06438 | IL2RB | 87.5529 | 58.3686 | 7 | 7 |

Table 9D: Compound Connections

| | | | score_best6 | | | |
|---|---|---|---|---|---|---|
| | cmap_iname | score_best4 | Connectivity | ncell | nsig | |
| cmap_id | cmap ID | Compound | score | score_best6 | ncell | nsig |
| 1 | BRD- | MG-132 | 98.1819 | 85.741 | 9 | 35 |
| | K60230970 | | | | | |
| 2 | BRD- | bortezomib | 96.6906 | 74.939 | 9 | 24 |
| | K88510285 | | | | | |
| 3 | BRD- | NSC-3852 | 96.3535 | 83.6295 | 9 | 13 |
| | K13169950 | | | | | |
| 4 | BRD- | MLN-2238 | 96.1711 | 82.8487 | 9 | 22 |
| | K78659596 | | | | | |
| 5 | BRD- | NSC-632839 | 95.9782 | 91.5581 | 9 | 17 |
| | K37392901 | | | | | |
| 6 | BRD- | MD-II-008-P | 95.4641 | 81.0888 | 9 | 13 |
| | K09854848 | | | | | |
| 7 | BRD- | MD-049 | 94.9585 | 92.4744 | 8 | 12 |
| | K74797618 | | | | | |
| 8 | BRD- | dasatinib | 94.6238 | 76.8325 | 9 | 14 |
| | K49328571 | | | | | |
| 9 | BRD- | manumycin-a | 94.3796 | 87.2161 | 9 | 16 |
| | A77216878 | | | | | |
| 10 | BRD- | BRD-K30381304 | 94.2229 | 51.404 | 6 | 7 |
| | K30381304 | | | | | |
| 11 | BRD- | NSC-632839 | 93.8831 | 85.4549 | 9 | 33 |
| | K74402642 | | | | | |
| 12 | BRD- | arachidonyl- | 93.4499 | 85.8039 | 9 | 21 |
| | K07303502 | trifluoro-methane | | | | |
| 13 | BRD- | CT-200783 | 93.4331 | 88.3693 | 9 | 17 |
| | K02822062 | | | | | |
| 14 | BRD- | BRD-K00910650 | 93.0707 | 62.0471 | 7 | 10 |
| | K00910650 | | | | | |

Table 9E: Consensus Knockdown Connections

| | cmap_iname | score_best4 | score_best6 | ncell | nsig | |
|---|---|---|---|---|---|---|
| cmap_id | cmap_id | cmap_iname | score_best4 | score_best6 | ncell | nsig |
| 1 | CGS001-596 | BCL2 | 100 | 100 | 9 | 11 |
| 2 | CGS001-116443 | GRIN3A | 99.155 | 89.8453 | 8 | 8 |
| 3 | CGS001-5573 | PRKAR1A | 98.7339 | 94.993 | 9 | 11 |
| 4 | CGS001-5291 | PIK3CB | 98.529 | 88.6891 | 9 | 12 |
| 5 | CGS001-2146 | EZH2 | 98.0261 | 92.5674 | 9 | 12 |
| 6 | CGS001-2065 | ERBB3 | 97.9618 | 93.1415 | 9 | 11 |
| 7 | CGS001-50 | ACO2 | 97.4296 | 77.5259 | 8 | 10 |
| 8 | CGS001-431707 | LHX8 | 97.2318 | 80.3358 | 8 | 8 |
| 9 | CGS001-1026 | CDKN1A | 97.1391 | 92.0215 | 9 | 11 |
| 10 | CGS001-285220 | EPHA6 | 97.0154 | 94.465 | 9 | 9 |
| 11 | CGS001-11200 | CHEK2 | 96.9527 | 92.9928 | 9 | 12 |
| 12 | CGS001-5261 | PHKG2 | 96.9411 | 82.7127 | 9 | 11 |
| 13 | CGS001-205 | AK4 | 96.9283 | 92.4352 | 8 | 9 |
| 14 | CGS001-3480 | IGF1R | 96.7308 | 71.4803 | 9 | 11 |
| 15 | CGS001-5603 | MAPK13 | 96.707 | 92.8056 | 9 | 11 |
| 16 | CGS001-1111 | CHEK1 | 96.4037 | 92.6321 | 9 | 11 |
| 17 | CGS001-55526 | DHTKD1 | 95.8164 | 76.0863 | 7 | 7 |
| 18 | CGS001-8536 | CAMK1 | 95.6308 | 87.1889 | 9 | 9 |
| 19 | CGS001-1024 | CDK8 | 95.5876 | 88.1505 | 9 | 12 |
| 20 | CGS001-2521 | FUS | 95.5595 | 78.9608 | 8 | 9 |
| 21 | CGS001-5295 | PIK3R1 | 95.2092 | 77.9855 | 9 | 11 |
| 22 | CGS001-7057 | THBS1 | 95.1289 | 84.1052 | 9 | 11 |
| 23 | CGS001-5727 | PTCH1 | 95.1153 | 77.522 | 9 | 11 |

Table 9F: Overexpression Connections

| | cmap_iname | score_best4 | score_best6 | ncell | nsig | |
|---|---|---|---|---|---|---|
| cmap_id | cmap_id | cmap_iname | score_best4 | score_best6 | ncell | nsig |
| 1 | ccsbBroad304_06837 | RAF1 | 85.8473 | 64.2506 | 8 | 10 |
| 2 | ccsbBroad304_07304 | TNFRSF10B | 82.395 | 56.6417 | 8 | 8 |
| 3 | ccsbBroad304_01579 | SOX2 | 80.1266 | 64.8673 | 8 | 16 |
| 4 | ccsbBroad304_06992 | SRC | 78.7762 | 60.0776 | 7 | 9 |
| 5 | ccsbBroad304_06681 | P2RY2 | 77.6152 | 39.3625 | 7 | 7 |
| 6 | ccsbBroad304_06179 | ERG | 76.9973 | 62.1321 | 8 | 8 |
| 7 | ccsbBroad304_01186 | PHB | 76.6918 | 43.7032 | 6 | 6 |
| 8 | ccsbBroad304_06542 | LTBR | 76.6616 | 60.8568 | 9 | 9 |
| 9 | ccsbBroad304_04404 | TUBB6 | 75.7696 | −666 | 4 | 4 |
| 10 | ccsbBroad304_03197 | NDUFA13 | 75.416 | 29.8829 | 6 | 6 |
| 11 | ccsbBroad304_02451 | HOXB13 | 75.39 | 50.26 | 8 | 8 |
| 12 | ccsbBroad304_00499 | ELK3 | 75.0227 | 50.7024 | 7 | 7 |
| 13 | ccsbBroad304_02012 | FADD | 74.8586 | 49.9057 | 6 | 6 |
| 14 | ccsbBroad304_00163 | PRDM1 | 74.8019 | 57.12 | 8 | 8 |

Derived from Connectivity Map 02 database (portals.broadinstitute.org/cmap/).

### Example 6: FPS-Based Systematic Ex Vivo Assessment of Clinical Liver Tissues Identifies Combination Anti-Fibrotic Therapies

Multiple candidate anti-fibrotic targets/agents have been proposed in experimental studies, but their clinical relevance is unclear without evaluation in liver disease patients. The FPS modulation by BCL2 inhibition in clinical PCLS suggests that FPS can serve as a readout to evaluate clinically relevant anti-fibrotic effect in pre-clinical models. To systematically explore the idea, assessment was done in a set of experimental anti-fibrotic agents in ex vivo culture of PCLS tissues (with preserved multi-cell-type tissue microenvironment) from 78 chronic liver disease patients. The tested agents include various classes of compounds: inhibitors of fibrogenic cellular signaling, i.e., TGF-β pathway (galunisertib), epidermal growth factor (EGF) pathway (erlotinib), and lysophosphatidic acid (LPA) pathway (AM095); CMap-derived BCL2 antagonists (MG-132, bortezomib); a dual C-C chemokine receptor type 2/5 (CCR2/CCR5) inhibitor evaluated for treatment of NASH fibrosis (cenicriviroc); anti-diabetics that suppress fibrogenesis as one of their pleiotropic effects (pioglitazone, metformin); a green tea catechin shown to inhibit liver fibrosis in our recent pre-clinical study (epigallocatechin gallate [EGCG]); epigenetic modulators of PLS (I-BET 151, JQ1); CMap-derived PLS-modulating generic drugs (captopril, nizatidine) (Table 2). After 24 h of culture with the agents or vehicle controls, reduction of FPS-based prognostic risk level was examined, i.e., suppression of the high-risk genes and/or induction of low-risk genes jointly quantified as Combined Enrichment Score (CES). The CES-based FPS response was observed in 31% to 88% of the patients for the agents treated in more than five patients (FIG. 5A, Table 10), suggesting that clinical response is heterogeneous across patients and the ex vivo assessment may inform clinical response to the agents. At the drug level, modulation of each individual FPS gene (target FPS gene) varies across the agents, while the targeted genes are similar among subsets of the agents, suggesting that the agents elicit anti-fibrotic effect via shared or unique targets in the FPS (FIG. 5B).

TABLE 10
FPS response rate in organotypic ex vivo culture of clinical PCLS tissues.

| | | | PLS response |
|---|---|---|---|
| Drug | No. patients tested | FPS response rate | rate |
| Galunisertib | 13 | 6 (46%) | 7 (53.85%) |
| Erlotinib | 25 | 11 (44%) | 13 (52%) |
| AM095 | 29 | 11 (38%) | 8 (27.59%) |
| MG132 | 8 | 6 (75%) | 3 (37.5%) |
| Bortezomib | 7 | 4 (57%) | 5 (71.43%) |
| Cenicriviroc | 13 | 4 (31%) | 7 (53.85%) |
| Pioglitazone | 10 | 6 (60%) | 7 (70%) |
| Metformin | 8 | 7 (88%) | 6 (75%) |
| Epigallocatechin | 11 | 7 (64%) | 9 (81.82%) |
| gallate | | | |
| Nizatidine | 6 | 3 (50%) | 3 (50%) |

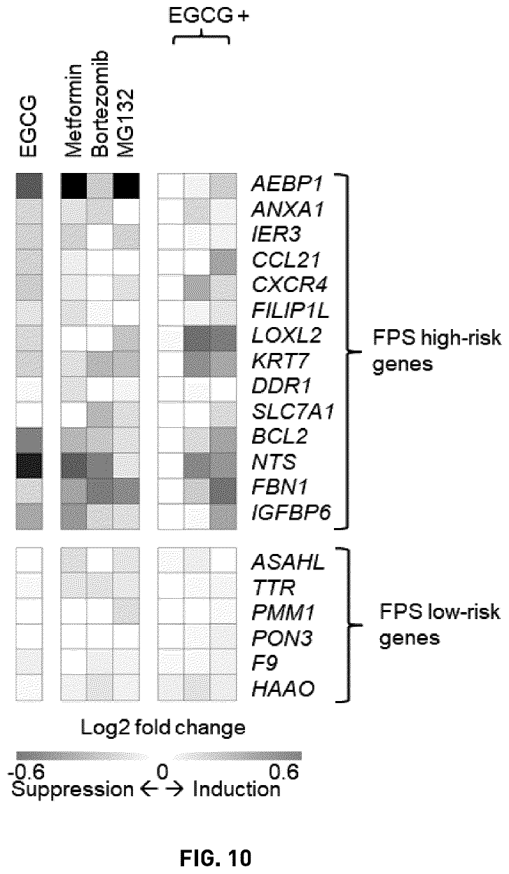

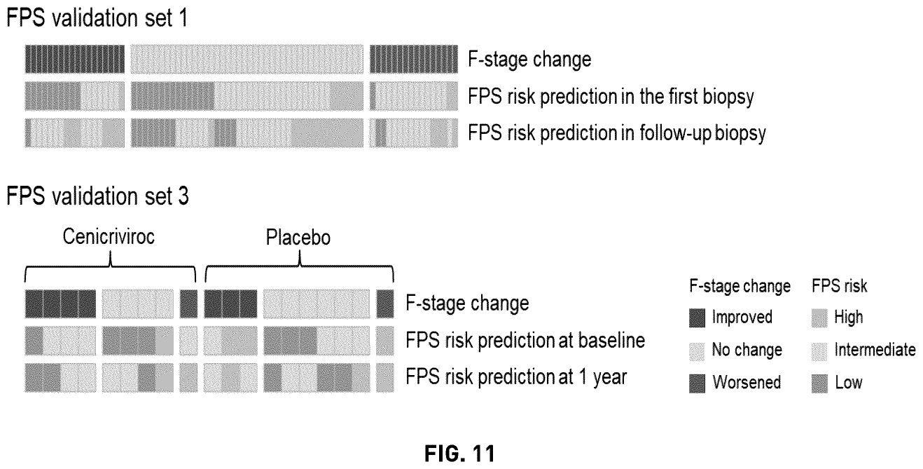

The target FPS genes are shared among agents in the same class of compounds such as MG132 and bortezomib, eliciting similar suppression of high-risk FPS genes, CCL21, BCL2, and IGFBP6. In contrast, agents with distinct mechanism of action such as galunisertib, AM095, and metformin showed similarly striking suppression of SLC7A1 (also known as CAT1), a recently identified anti-fibrotic target. These results demonstrate that the molecular-signature-based systematic evaluation enables unbiased identification of anti-fibrotic agents and their specific targets. In addition, the diverse target FPS genes across the agents suggest opportunities of combining multiple agents that complementarily target FPS member genes for synergistic and enhanced anti-fibrotic effect. To test this idea, synergistic effect of the agents to shift the FPS from high- to low-risk pattern was computationally inferred, i.e., suppression of the high-risk genes and/or induction of the low-risk genes (see materials and methods). We identified four candidate combinations based on EGCG as a backbone (FIG. 5C, FIG. 5D). The predicted combinatorial anti-fibrotic effect was validated in ex vivo culture of a PCLS tissue from a chronic hepatitis C patient with F2 fibrosis. It was observed that addition of bortezomib and MG132, but not metformin, to EGCG resulted in substantially reduced expression of genes encoding extracellular matrix proteins (e.g., COL1A1, HAS2) and fibrogenic drivers (e.g., PDGFRB, TGFB1, NOTCH1, LPAR1), confirming their synergistic anti-fibrotic effect in patient-derived fibrotic liver tissue (FIG. 5E). The high-risk FPS genes were more broadly suppressed with the combinations compared to mono-therapies (FIG. 10). These results collectively demonstrate that FPS-based analysis of clinical PCLS tissues enables ex vivo testing of candidate anti-fibrotic agents in clinical liver tissues and identifies rational, molecular-targeted combinatorial anti-fibrotic therapies. We further confirmed that combination of EGCG and bortezomib resulted in suppression of broader fibrosis-related genes compared to mono-therapies in a patient-derived liver spheroid (FIG. 5F). We recently developed a PLS-inducible cell culture model (cPLS system) for high-throughput screening of HCC chemopreventive agents.³⁷ We confirmed that pharmacological FPS modulation similar to that in the PCLS culture was observed in the simple and robust cell culture system (FIG. 5G), indicating that FPS-based high-throughput compound screening is feasible to efficiently identify new anti-fibrotics with prognostic impact quantitatively measured by FPS modulation.

### Example 7: FPS and Global Transcriptome Profiles to Monitor Anti-Fibrogenic Activity of Cenicriviroc in NASH Patients

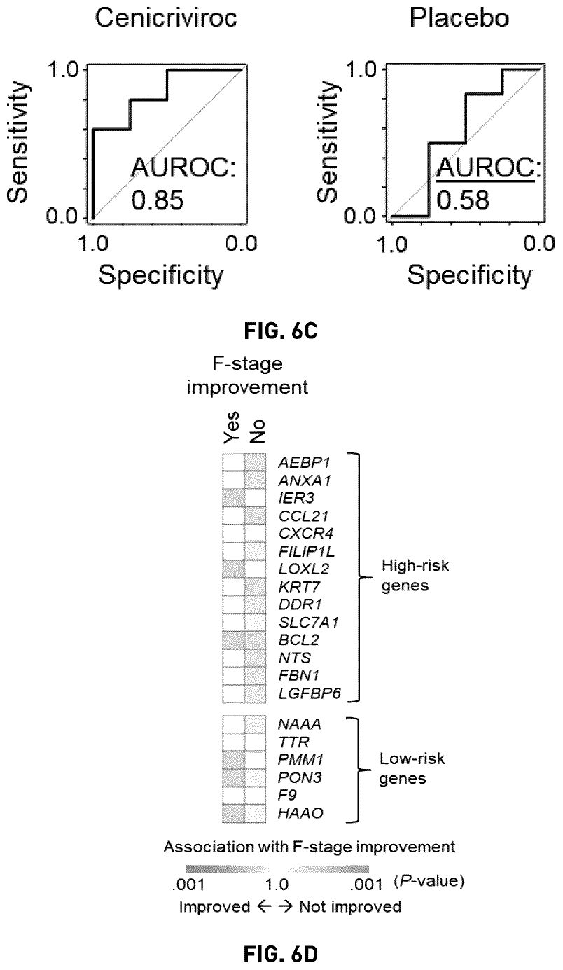

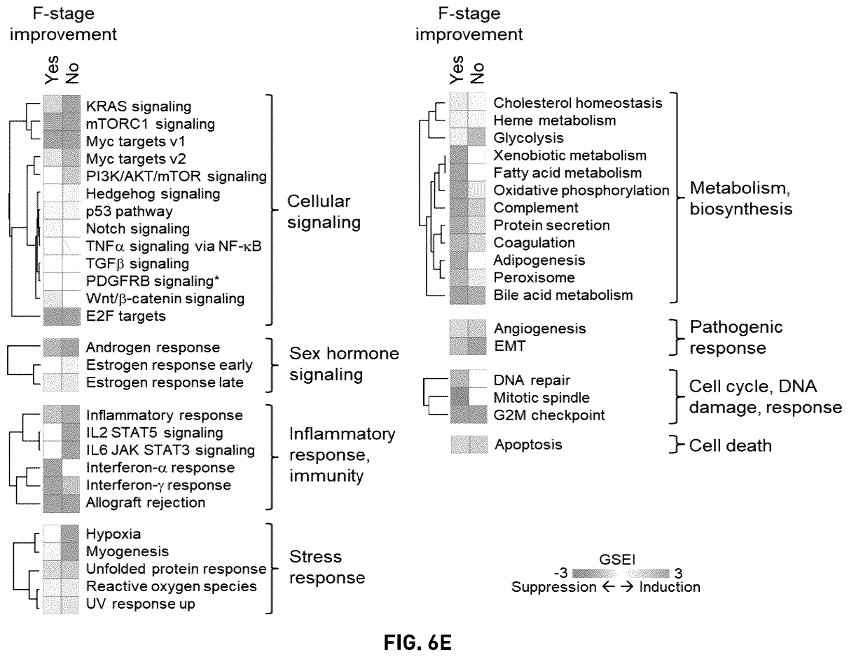

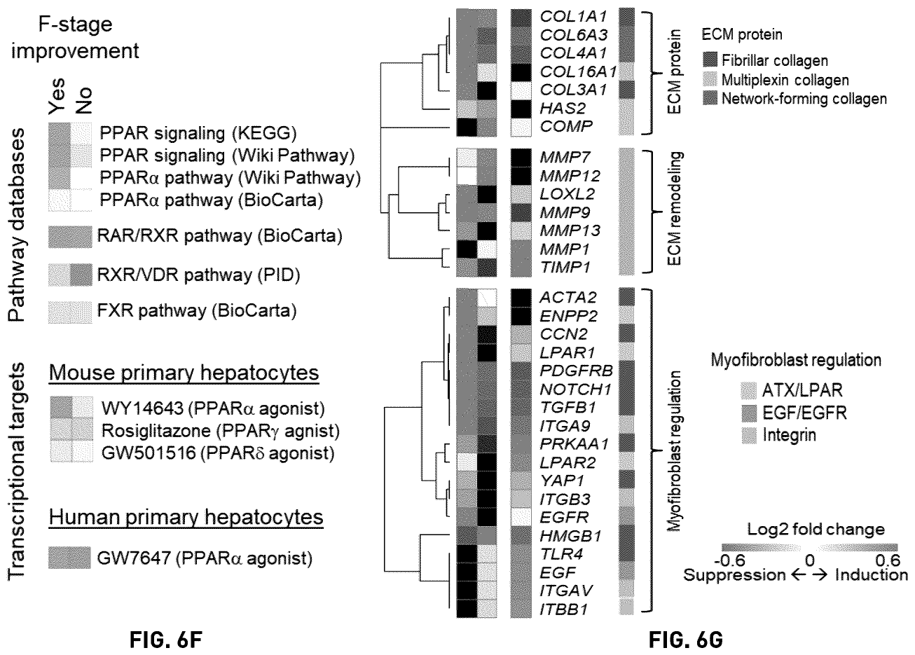

In the recent phase Ilb CENTAUR trial, 1-year treatment with a dual CCR2/CCR5 inhibitor, cenicriviroc, resulted in improved histological fibrosis that persisted another year in NASH patients with F1 to F3 fibrosis. Profiling was done on the hepatic transcriptomes from these patients to analyze the therapeutic modulation of FPS and global molecular pathways using paired pre- and post-treatment biopsy tissues from 9 cenicriviroc- and 10 placebo-treated patients. In the cenicriviroc and placebo arms, 4 and 3 patients showed improvement of fibrosis in the year 1 biopsy, respectively (FIG. 6A). Despite the small sample size not intended to assess FPS, post-treatment FPS modulation measured by CES showed a trend of association with the improved fibrosis in the cenicriviroc arm, while it was not obvious in the placebo arm (FIG. 6B, FIG. 6C). These results may suggest that FPS modulation is more strongly correlated with pharmacological fibrosis improvement compared to spontaneous change, especially in such short timeframe (1 year). Of note, the proportion of patients with substantial CES reduction (i.e., FPS response) was comparable between the CENTAUR trial (22%) and the ex vivo PCLS tissue culture (31%) (FIG. 5A), suggesting the potential clinical utility of the short-term ex vivo PCLS culture to predict clinical anti-fibrotic responses to the therapy.

In the cenicriviroc arm, the FPS genes were suppressed or unchanged in patients who showed histological fibrosis improvement, whereas the genes were generally induced in patients with no fibrosis improvement (FIG. 6D). A comprehensive assessment of molecular pathway modulation in global hepatic transcriptome revealed that the fibrosis responders showed suppression of specific fibrogenic pathways, which was not observed in the non-responders (FIG. 6E, Table 11). The E2F signaling, previously implicated in chemically-or physiologically-induced liver fibrosis in mice, was most strikingly suppressed, followed by the Wnt/β-catenin signaling only in the responders. Interestingly, other well-known fibrogenic pathways, i.e., TGF-β and platelet derived growth factor receptor β (PDGFRB) pathways were unchanged, suggesting that these pathways are irrelevant to anti-fibrotic effect of cenicriviroc. This finding suggests that the E2F pathway may be an indicator of cenicriviroc response and represent a target to address absence of anti-fibrotic response.

Nuclear receptor signaling pathways such as peroxisome proliferator-activated receptor (PPAR), retinoid X receptor (RXR), retinoic acid receptor (RAR), and farnesoid X receptor (FXR) have been explored as therapeutic targets in NASH. In recent clinical trials, combination therapies that involve agonists of the pathways have been actively evaluated to achieve clinically meaningful anti-fibrotic effect in NASH patients. Of note, the fibrosis responders are characterized by enrichment of PPAR pathways after the 1-year cenicriviroc treatment, whereas the non-responders lack such modulation of the pathway (FIG. 6F). There was no obvious induction of nor difference in RXR, RAR, and FXR pathways in both responders and non-responders. Assessment of experimentally-defined transcriptional target gene signatures of α, δ, and γ agonists revealed that PPAR α is dominantly induced by cenicriviroc in mouse primary hepatocyte (see Table 11). Induction of pharmacological PPARa target genes in the fibrosis responders was also confirmed in human primary hepatocytes. Combination of cenicriviroc and a PPARα agonist, bezafibrate, led to reduced expression of genes encoding extracellular matrix proteins in a patient-derived liver spheroid (FIG. 6G). In summary, these findings suggest that combination with PPARα agonists may improve the anti-fibrotic efficacy of cenicriviroc in non-responders, which warrants further assessment in future studies.

TABLE 11
Molecular pathways modulated by cenicriviroc in
NASH patients according to fibrosis improvement.
Table 11A: General molecular pathways (Hallmark set, curated pathway targets)

| | | | Fibrosis | | | Fibrosis | |
|---|---|---|---|---|---|---|---|
| | | | improved | | | not improved | |
| Database | Gene set | NES | P | FDR | NES | P | FDR |
| Cellular | KRAS signaling | 1.31 | .041 | .097 | 1.63 | <.001 | .011 |
| signaling | | | | | | | |
| | mTORC1 signaling | 1.71 | .002 | .007 | 1.93 | <.001 | .001 |
| | Myc targets v1 | 1.56 | <.001 | .018 | 2.19 | <.001 | <.001 |
| | Myc targets v2 | 1.26 | .103 | .135 | 1.55 | .007 | .019 |
| | PI3K/AKT/mTOR | 0.91 | .639 | .713 | 1.43 | .018 | .048 |
| | signaling | | | | | | |
| | Hedgehog signaling | −0.65 | .945 | 1.0 | 1.05 | .368 | .462 |
| | p53 pathway | 1.00 | .438 | .547 | 1.03 | .371 | .488 |
| | Notch signaling | 1.00 | .451 | .561 | 0.84 | .696 | .910 |
| | TNFα signaling via | 0.96 | .558 | .635 | 0.96 | .542 | .664 |
| | NF-κB | | | | | | |
| | TGFβ signaling | 0.81 | .791 | .875 | −0.70 | .942 | .977 |
| | Wnt/β-catenin | −1.07 | .355 | .570 | 0.87 | .694 | .902 |
| | signaling | | | | | | |
| | E2F targets | −1.36 | .012 | .169 | 1.88 | <.001 | .001 |
| Sex hormone | Androgen response | 1.52 | .006 | .024 | 1.63 | <.001 | .011 |
| signaling | | | | | | | |
| | Estrogen response | −0.79 | .938 | 1.0 | 1.07 | .303 | .424 |
| | early | | | | | | |
| | Estrogen response | 1.12 | .204 | .335 | 1.18 | .132 | .226 |
| | late | | | | | | |
| Inflammatory | Inflammatory | 1.38 | .014 | .060 | 1.61 | .003 | .012 |
| response, | response | | | | | | |
| immunity | | | | | | | |
| | IL2 STAT5 signaling | 0.89 | .752 | .752 | 1.70 | <.001 | .007 |
| | IL6 JAK STAT3 | 0.95 | .550 | .635 | 1.82 | <.001 | .002 |
| | signaling | | | | | | |
| | Interferon-α response | 2.12 | <.001 | <.001 | 0.83 | .799 | .896 |
| | Interferon-γ response | 2.07 | <.001 | <.001 | 1.39 | .015 | .060 |
| | Allograft rejection | 1.83 | <.001 | .001 | 2.25 | <.001 | <.001 |
| Metabolism, | Cholesterol | 1.19 | .175 | .222 | 0.99 | .467 | .577 |
| biosynthesis | homeostasis | | | | | | |
| | Heme metabolism | −0.98 | .500 | .724 | −0.95 | .589 | .963 |
| | Glycolysis | 1.10 | .250 | .367 | 1.43 | .009 | .050 |
| | Xenobiotic | 1.66 | <.001 | .011 | 0.79 | .936 | .917 |
| | metabolism | | | | | | |
| | Fatty acid | 2.22 | <.001 | <.001 | 0.73 | .973 | .957 |
| | metabolism | | | | | | |
| | Oxidative | 2.49 | <.001 | <.001 | 1.16 | .158 | .264 |
| | phosphorylation | | | | | | |
| | Complement | 2.07 | <.001 | <.001 | 1.37 | .024 | .065 |
| | Protein secretion | 1.57 | .002 | .016 | 1.30 | .073 | .104 |
| | Coagulation | 1.57 | .006 | .018 | 1.33 | .051 | .087 |
| | Adipogenesis | 1.60 | .002 | .016 | −0.72 | .986 | 1.0 |
| | Peroxisome | 1.49 | .006 | .029 | −1.03 | .402 | .940 |
| | Bile acid metabolism | 1.62 | <.001 | .015 | −1.36 | .029 | .179 |
| Stress | Hypoxia | 0.97 | .521 | .624 | 1.67 | <.001 | .010 |
| response | | | | | | | |
| | Myogenesis | −0.95 | .583 | .727 | 1.73 | <.001 | .006 |
| | Unfolded protein | 1.34 | .045 | .084 | 1.41 | .017 | .051 |
| | response | | | | | | |
| | Reactive oxygen | 1.07 | .340 | .404 | 1.24 | .158 | .166 |
| | species | | | | | | |
| | UV response up | 1.12 | .222 | .349 | 1.24 | .101 | .165 |
| Pathogenic | Angiogenesis | 1.47 | .045 | .032 | 1.64 | .022 | .012 |
| response | | | | | | | |
| | Epithelial | 1.41 | .010 | .048 | 1.98 | <.001 | <.001 |
| | mesenchymal | | | | | | |
| | transition | | | | | | |
| Cell cycle, | DNA repair | 1.44 | .006 | .039 | 0.85 | .803 | .920 |
| DNA damage, | | | | | | | |
| response | | | | | | | |
| | Mitotic spindle | −1.61 | <.001 | .055 | 0.82 | .916 | .887 |
| | G2M checkpoint | −1.33 | .015 | .167 | 2.00 | <.001 | <.001 |
| Cell death | Apoptosis | 1.32 | .038 | .094 | 1.38 | .024 | .066 |

Table 11B: Nuclear receptor signaling (curated pathway members)

| | | Fibrosis | | | Fibrosis | |
|---|---|---|---|---|---|---|
| | | improved | | | not improved | |
| Gene set | NES | P | FDR | NES | P | FDR |
| PPAR signaling | 1.81 | .002 | .016 | −0.88 | .713 | 1.0 |
| PPAR signaling | 1.70 | .002 | .032 | −1.00 | .453 | .901 |
| PPARα pathway | 1.55 | .023 | .092 | −0.62 | .961 | .965 |
| PPARα pathway | 0.92 | .585 | .666 | −0.77 | .862 | .931 |
| RAR/RXR | −1.50 | .058 | .133 | −1.49 | .065 | .202 |
| pathway | | | | | | |
| FXR pathway | −1.06 | .418 | .359 | −1.04 | .437 | .948 |
| RXR/VDR | −1.10 | .326 | .583 | −1.71 | .013 | .092 |
| pathway | | | | | | |

Table 11C: iLINCS (genetic perturbation signature database)

| | | | Fibrosis | | Fibrosis | | |
|---|---|---|---|---|---|---|---|
| | | | improved | | not improved | | |
| Database | Gene set | NES | P | FDR | NES | P | FDR |
| | PDGFRB target | −0.83 | .847 | .823 | −0.79 | .908 | .892 |
| | gene signature | | | | | | |
| PPAR | WY14643 | 1.56 | <.001 | .056 | 1.17 | .193 | |
| agonists | (PPARa agonist) | | | | | | |
| on mouse | | | | | | | |
| primary | | | | | | | |
| hepatocytes | | | | | | | |
| (GSE27948) | | | | | | | |
| | ROSIGLITAZONE | 1.36 | .040 | .140 | −1.19 | .150 | .208 |
| | (PPARg agonist) | | | | | | |
| | GW501516 | 1.16 | .197 | .303 | −0.92 | .631 | .617 |
| | (PPARd agonist) | | | | | | |
| PPAR | GW7647 | 1.84 | <.001 | <.001 | −1.56 | .010 | .010 |
| agonist on | (PPARa agonist) | | | | | | |
| human | | | | | | | |
| primary | | | | | | | |
| hepatocytes | | | | | | | |
| (GSE53399) | | | | | | | |
| Immune cell | Macrophage | 1.42 | .068 | .245 | 1.70 | .004 | .020 |
| signature | (M1) | | | | | | |
| (GSE65136) | | | | | | | |
| | Macrophage | 2.66 | <.001 | .038 | 1.74 | .007 | .016 |
| | (M2) | | | | | | |
| | Monocyte | 2.81 | .002 | .036 | 1.50 | .028 | .077 |
| | Neutrophil | 1.87 | .010 | .077 | 1.42 | .032 | .102 |
| | NK cell (rest) | 1.12 | .267 | .451 | 1.76 | <.001 | .015 |
| | DC (activate) | 1.35 | .108 | .278 | 1.75 | .004 | .016 |
| | B cell (naïve) | −0.52 | .993 | .976 | 1.70 | .002 | .021 |
| | Memory B cell | −0.72 | .857 | 1.0 | 1.67 | .004 | .024 |
| | Plasma cell | 0.98 | .441 | .612 | 1.46 | .050 | .090 |
| | CD4 T cell | −0.57 | .981 | 1.0 | 1.93 | <.001 | .003 |
| | (naïve) | | | | | | |
| | CD8 T cell | 1.13 | .253 | .447 | 2.31 | <.002 | <.001 |
| | Memory T cell | 0.80 | .744 | .819 | 2.25 | <.003 | <.001 |
| | (rest) | | | | | | |
| | Regulatory | −0.99 | .409 | .983 | 1.99 | <.004 | .001 |
| | T cell | | | | | | |
| | T follicular | 1.22 | .182 | .357 | 1.85 | .002 | .006 |
| | helper cell | | | | | | |
| | Gamma delta | 1.09 | .298 | .484 | 1.86 | .002 | .006 |
| | T cell | | | | | | |
| | Mast cell (active) | 1.22 | .206 | .358 | 1.50 | .041 | .076 |
| | Mast cell (rest) | −1.59 | .046 | .388 | 1.71 | .002 | .021 |

NES, normalized enrichment score; FDR, false discovery rate.
Click hyperlink for details about each gene set.

### Example 8: Serum-Based FPS for Non-Invasive Assessment of Fibrosis Progression Risk

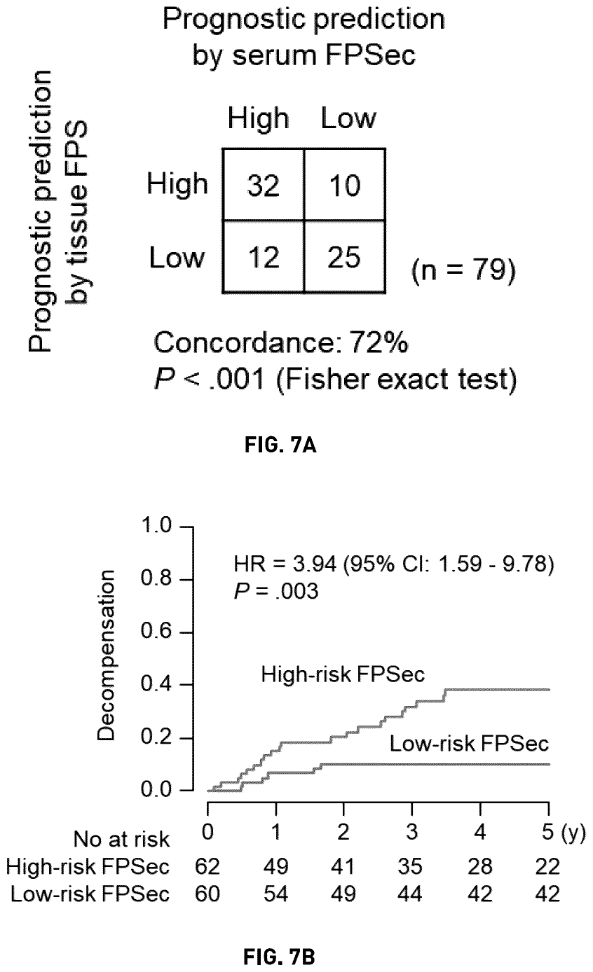

Despite the encouraging prognostic capability of FPS, requirement of liver biopsy tissue will limit its clinical applicability. It has been recently demonstrated that tissue transcriptome signature can be translated into serum-protein-based surrogate biomarker by utilizing our in silico pipeline, TexSEC (www.tex-sec.app). By utilizing the established pipeline, a 7-protein FPS surrogate, Fibrosis Progression Secretome signature (FPSec) was defined, which showed significant correlation with the FPS gene expression (FDR <0.001) (refer Table 4). FPSec was tested using archived serum samples from a cohort of 79 Japanese cirrhosis patients with mixed etiologies. As expected, it was observed significant concordance between the tissue-mRNA- and serum-protein-based prognostic risk prediction (p<0.001, Fisher's exact test) (FIG. 7A). It was further tested if the FPSec predicts development of hepatic decompensation as a measure of fibrotic disease progression and a surrogate of fibrosis progression in 122 patients with compensated cirrhosis from mixed etiologies analyzed in our previous study (FPSec validation set) (Table 1). During a median follow-up of 5.5 years (IQR, 1.8-12.1 years), 29 patients developed hepatic decompensation. High-risk FPSec (n=62, 51%) was significantly associated with incidence of hepatic decompensation (hazard ratio [HR], 3.94; 95% CI, 1.59-9.78), which is superior to the prognostic association of PLSec reported in our previous study (HR, 3.51; 95% CI, 1.61-7.63) (FIG. 7B), and remained significant even after adjustment for a clinically available score, ALBI-FIB-4 score, (adjusted HR, 3.00; 95% CI, 1.16-7.79) (Table 5). FPSec showed superior goodness of fit compared to ALBI-FIB-4 score (likelihood ratio test P<0.001), and improved goodness of fit of ALBI-FIB-4 alone (likelihood ratio test P=0.02). These results warrant further validation of FPSec as a non-invasive biomarker to assess risk of future fibrosis progression. In addition, given that the FPS-based risk status could change overtime spontaneously or in response to lifestyle or therapeutic interventions (FIG. 5A (FIG. 5)), this blood-based assay will enable more detailed time-series analysis to gain insight about how the molecular risk of fibrosis progression evolves over the natural history of chronic liver diseases.

### Example 9: Summary of results

The lengthy process of liver fibrosis progression has hampered discovery and validation of biomarkers predictive of long-term fibrosis progression. To overcome the change, patient cohorts with naturally-occurring (i.e., HIV infection) and iatrogenic (i.e., immunosuppressant use post transplantation) immune-suppressive conditions that accelerate fibrosis progression in the discovery of the FPS was utilized. The significant prognostic association for both PLS and FPS supports their utility as surrogate biomarkers to reliably estimate future fibrosis progression from the earliest stages with no to minimal fibrous tissue across patients, representing the major liver disease etiologies, i.e., chronic HCV infection and NAFLD. The clinical impact of such biomarkers to identify a subset of patients with rapid disease progression cannot be overemphasized, given the vast size of the population with early-stage chronic liver disease, the majority of which will be indolent. The disclosed FPS can help optimize the allocation of limited medical resources to the at-risk patients.

Serum-based PLS can monitor dynamic change of prognostic risk level over the course of antiviral treatment in patients with chronic hepatitis C and this change is correlated with future disease progression. This suggests that the signature can be used as a surrogate endpoint in clinical trials of anti-fibrotic agents to estimate their long-term prognostic impact within the typical timeframe of clinical trial and study (e.g., 5 years). In addition, a high-risk FPS may be used as a selection biomarker to indicate anti-fibrotic therapies and/or to guide patient enrollment in anti-fibrotic clinical trials. The results disclosed herein demonstrated that similar therapeutic modulation of FPS can be monitored even in the short-term ex vivo treatment of clinical PCLS. This encouraging finding indicates that rapid ex vivo assessment may serve as “avatar” for each individual patient to predict anticipated therapeutic benefit prior to initiation of the therapy. Furthermore, ex vivo testing in a cohort of patients enables exploration of response-associated clinical factors, which may guide study design of subsequent clinical trials. Collectively, FPS should therefore facilitate clinical testing of experimental anti-fibrotic agents.

The FPS also provides clues to genetic drivers of fibrosis progression/resolution as targets for new anti-fibrotic strategies and/or to resolve resistance to existing therapies. The confirmed prognostic association in multiple clinical cohorts would support confidence in their clinical relevance. Genetic targeting has been increasingly recognized as a clinically viable therapeutic option with the recent FDA approval of oligonucleotide-based, liver-directed therapy. Hepatic-cell-type-specific delivery of gene-targeting reagents is now also feasible. The disclosed gene-signature-based integrative systems biology approach also identified small molecular compounds that mimic genetic targeting toward the FPS member genes. Furthermore, the characterization of genetic targets specific to each compound enables systematic identification of rational combination anti-fibrotic therapies as demonstrated by the example of EGCG-based combinations with BCL2-targeting compounds. It may help maximize anti-fibrotic efficacy, while mitigating toxicity by reduced dosing for each agent in the combinations. Interestingly, the E2F pathway is the major anti-fibrotic target of cenicriviroc.

The capability of the disclosed methods to assay serum samples will enable more flexible testing for expanded clinical scenarios such as longitudinal repeated measurements. In summary, the present disclosure provides a new strategy of prognostic-risk-based individualized patient management and biomarker-guided anti-fibrotic drug development to facilitate clinical translation of promising experimental anti-fibrotic agents. The disclosed methods have an integrative strategy that will contribute to transformative improvement of the dismal prognosis of the patients with chronic fibrotic liver diseases.

What is claimed is:

1. A method of predicting risk for liver fibrosis progression in a subject comprising determining a fibrosis progression signature (FPSec) score for the subject, wherein the subject is having or is suspected of having a disease, a condition, or any combination thereof that predisposes the subject to liver fibrosis.

2. The method of claim 1 further comprising a method of obtaining the FPSec score for the subject, wherein the method of obtaining the FPS score comprises:
(a) obtaining a sample of blood from the subject;
(b) subjecting the sample to a multi-analyte profiling assay for protein quantification of one or more proteins, wherein the proteins comprise angiogenin, matrix metallopeptidase 7 (MMP-7), insulin like growth factor binding protein 7 (IGFBP-7), protein S (PROS1), vascular cell adhesion molecule 1 (VCAM-1), interleukin 6 (IL-6) C-C motif chemokine ligand 21 (CCL-21), or any combination thereof, to obtain a protein quantification measurement for each of the one or more proteins;
(c) normalizing the protein quantification measurements of angiogenin, MMP-7, IGFBP-7, protein S, VCAM-1, IL-6, and/or CCL-21 to median fluorescent intensity to obtain a normalized protein quantification measurement for each of the one or more proteins; and
(d) converting the normalized protein quantification measurements of angiogenin, MMP-7, IGFBP-7, protein S, VCAM-1, IL-6, and/or CCL-21 into an aggregated score, wherein the aggregated score is the FPSec score.

3. The method of claim 1 or 2, wherein the subject is predicted to be at low risk for liver fibrosis progression if the FPSec score is below 3.

4. The method of claim 1 or 2, wherein the subject is predicted to be at high risk for liver fibrosis progression if the FPSec score is 3 or above.

5. The method of any one of claim 1 or 2, wherein the disease, condition, or combination thereof that predisposes the subject to liver fibrosis comprises chronic infection of hepatitis B virus (HBV), chronic infection of hepatitis C virus (HCV), non-alcoholic fatty liver disease (NAFLD), non-alcoholic steatohepatitis (NASH), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

6. The method of claim 2, further comprising diagnosing liver fibrosis in the subject.

7. The method of claim 6, wherein the method of diagnosing liver fibrosis in the subject comprises performing a liver biopsy, one or more blood tests to assess liver function, computed tomography, magnetic resonance imaging, or any combination thereof.

8. The method of claim 7, wherein the one or more blood tests performed to assess liver function comprises a measurement of alanine transaminase (ALT), aspartate transaminase (AST), alkaline phosphatase (ALP), albumin, bilirubin, gamma-glutamyltransferase (GGT), L-lactate dehydrogenase (LD), prothrombin time (PT), or any combination thereof.

9. The method of claim 2, further comprising administering one or more treatments of liver fibrosis to the subject.

10. The method of claim 9, wherein the one or more treatments of liver fibrosis comprises anti-fibrotic therapy.

11. The method of claim 10, wherein the anti-fibrotic therapy comprises administration of one or more drugs to the subject, wherein the drugs are selected from galunisertib, erlotinib, AM095, bortezomib, pioglitazone, metformin, epigallocatechin gallate (EGCG), I-BET 151, JQ1, captopril, and nizatidine (Selleck Chemicals), MG-132 cenicriviroc, and any combination thereof.

12. A method of determining a fibrosis progression signature (FPSec) score for a subject comprising:
(a) obtaining a sample of blood from the subject;
(b) subjecting the sample to a multi-analyte profiling assay for protein quantification of one or more proteins, wherein the proteins comprise angiogenin, matrix metallopeptidase 7 (MMP-7), insulin like growth factor binding protein 7 (IGFBP-7), protein S (PROS1), vascular cell adhesion molecule 1 (VCAM-1), interleukin 6 (IL-6) C-C motif chemokine ligand 21 (CCL-21) or any combination thereof, to obtain a protein quantification measurement for each of the one or more proteins;
(c) normalizing the protein quantification measurements of angiogenin, MMP-7, IGFBP-7, protein S, VCAM-1, IL-6, and/or CCL-21 to median fluorescent intensity to obtain a normalized protein quantification measurement for each of the one or more proteins; and
(d) converting the normalized protein quantification measurements of angiogenin, MMP-7, IGFBP-7, protein S, VCAM-1, IL-6, and/or CCL-21 into an aggregated score, wherein the aggregated score is the FPSec score.

13. The method of claim 12, wherein the subject is having or is suspected of having a disease, a condition, or a combination thereof that predisposes the subject to liver fibrosis.

14. The method of claim 13, wherein the disease, condition, or combination thereof that predisposes the subject to liver fibrosis comprises chronic infection of hepatitis B virus (HBV), chronic infection of hepatitis C virus (HCV), non-alcoholic fatty liver disease (NAFLD), non-alcoholic steatohepatitis (NASH), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

15. The method of any one of claims 12 to 14, wherein a subject having an FPSec score below 3 is at low risk for liver fibrosis progression.

16. The method of any one of claims 12 to 14, wherein a subject having an FPSec score of 3 or above is at high risk for liver fibrosis progression.

17. A diagnostic kit for determining a fibrosis progression signature (FPSec) score of a subject comprising one or more reagents for use in a multi-analyte profiling assay.

18. The diagnostic kit of claim 17, wherein the one or more reagents for use in a multi-analyte profiling assay comprises beads labeled with antibodies to angiogenin, matrix metallopeptidase 7 (MMP-7), insulin like growth factor binding protein 7 (IGFBP-7), protein S (PROS1), vascular cell adhesion molecule 1 (VCAM-1), interleukin 6 (IL-6), and/or C-C motif chemokine ligand 21 (CCL-21).

19. A method of treating liver fibrosis in a subject at high risk for liver fibrosis progression, the method comprising:
(a) determining if the subject is at high risk for liver fibrosis progression by:
(i) obtaining a sample of blood from the subject;
(ii) determining protein levels of at least two liver disease biomarkers in the sample wherein,
one of the at least two liver disease biomarkers is selected from vascular cell adhesion molecule 1 (VCAM-1), insulin-like growth factor-binding protein 7 (IGFBP-7), matrix metallopeptidase 7 (MMP-7), interleukin-6 (IL-6), and C-C motif chemokine ligand 21 (CCL-21); and
the other one of the at least two liver disease biomarkers is selected from angiogenin and protein S;
(iii) determining that the subject is at high risk for liver fibrosis progression if the one of the at least two liver disease biomarkers selected from VCAM-1, IGFBP-7, MMP-7, IL-6, and CCL-21 has a higher protein expression compared to a control, and the other one of the at least two liver disease biomarkers selected from angiogenin and protein S has a lower protein expression compared to a control,
wherein the control is a sample of blood from a subject known to not have any liver disease; and
(b) administering one or more treatments of liver fibrosis to the subject determined to be at high risk for liver fibrosis progression.

20. The method of claim 19, wherein the subject is at high risk for liver fibrosis progression if any one of VCAM-1, IGFBP-7, MMP-7, IL-6, and CCL-21 has a higher protein expression compared to the control and any one of angiogenin or protein S has a lower protein expression compared to the control.

21. The method of claim 20, wherein the subject is at high risk for liver fibrosis progression if VCAM-1, IGFBP-7, MMP-7, IL-6, and CCL-21 have a higher protein expression compared to the control and angiogenin and protein S have a lower protein expression compared to the control.

22. The method of any one of claims 19 to 21, wherein the protein level of at least two liver disease biomarkers is determined by one or more methods selected the group consisting of Western blotting, enzyme-linked immunosorbent assay (ELISA), multi-analyte profiling assay, mass spectrometry, HPLC, flow cytometry, fluorescence-activated cell sorting (FACS), liquid chromatography-mass spectrometry (LC/MS), immunoelectrophoresis, translation complex profile sequencing (TCP-seq), protein microarray, protein chip, capture arrays, reverse phase protein microarray (RPPA), two-dimensional gel electrophoresis or (2D-PAGE), functional protein microarrays, electrospray ionization (ESI), matrix-assisted laser desorption/ionization (MALDI), and any combination thereof.

23. The method of claim 22, wherein the protein level of at least two liver disease biomarkers is determined by ELISA or multi-analyte profiling assay.

24. The method of any one of claims 19 to 21, wherein the one or more treatments of liver fibrosis comprises an anti-fibrotic therapy.

25. The method of claim 24, wherein the anti-fibrotic therapy comprises administration of one or more drugs to the subject, wherein the drugs are comprised of galunisertib, erlotinib, AM095, bortezomib, pioglitazone, metformin, epigallocatechin gallate (EGCG), I-BET 151, JQ1, captopril, and nizatidine (Selleck Chemicals); MG-132, cenicriviroc, or any combination thereof.

26. A method of predicting risk for liver fibrosis progression in a subject comprising determining a fibrosis progression signature (FPS) score for the subject, wherein the subject is having or is suspected of having a disease, a condition, or a combination thereof that predisposes the subject to liver fibrosis.

27. The method of claim 26 further comprising a method of obtaining the FPS score for the subject, wherein the method of obtaining the FPS score comprises:
(a) obtaining a tissue sample from the subject;
(b) subjecting the tissue sample to a multi-analyte profiling assay for gene expression of one or more genes, wherein the genes comprise ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9, or any combination thereof, to obtain a gene expression measurement for each of the one or more genes;
(c) normalizing the gene expression measurements of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to expression levels of a control set of genes to obtain a gene expression profile of the subject; and
(d) converting the gene expression profile of the subject into an FPS score, wherein the FPS score is a numerical value corresponding to a similarity between the gene expression profile of the subject and a high-risk reference gene expression profile or a low-risk reference gene expression profile.

28. The method of claim 27, wherein the subject is predicted to be at low risk for liver fibrosis progression if the FPS score is less than −1.3013.

29. The method of claim 27, wherein the subject is predicted to be at intermediate risk for liver fibrosis progression if the FPS score is between −1.3013 and +1.3013.

30. The method of claim 27, wherein the subject is predicted to be at high risk for liver fibrosis progression if the FPS score is greater than +1.3013.

31. The method of claim 30, further comprising diagnosing liver fibrosis in the subject.

32. The method of claim 31, wherein diagnosing liver fibrosis in the subject comprises performing a liver biopsy, one or more blood tests to assess liver function, computed tomography, magnetic resonance imaging, or any combination thereof.

33. The method of claim 32, wherein the one or more blood tests performed to assess liver function comprises a measurement of alanine transaminase (ALT), aspartate transaminase (AST), alkaline phosphatase (ALP), albumin, bilirubin, gamma-glutamyltransferase (GGT), L-lactate dehydrogenase (LD), prothrombin time (PT), or any combination thereof.

34. The method of any one of claims 27 to 33, further comprising administering one or more treatments of liver fibrosis to the subject.

35. The method of claim 34, wherein the one or more treatments of liver fibrosis comprises anti-fibrotic therapy.

36. The method of claim 35, wherein the anti-fibrotic therapy comprises administration of one or more drugs to the subject, wherein the drugs are comprised of galunisertib, erlotinib, AM095, bortezomib, pioglitazone, metformin, epigallocatechin gallate (EGCG), I-BET 151, JQ1, captopril, and nizatidine (Selleck Chemicals); MG-132, cenicriviroc, or any combination thereof.

37. The method of any one of claim 1, 13, or 26, wherein the disease, condition, or combination thereof that predisposes the subject to liver fibrosis comprises chronic infection of hepatitis B virus (HBV), chronic infection of hepatitis C virus (HCV), non-alcoholic fatty liver disease (NAFLD), non-alcoholic steatohepatitis (NASH), hereditary hemochromatosis, type 2 diabetes, obesity, tobacco use, alcohol abuse, long-term anabolic steroid use, tyrosinemia, alpha-1-antitrypsin deficiency, porphyria cutanea tarda, glycogen storage diseases, Wilson disease, or any combination thereof.

38. A method of determining a fibrosis progression signature (FPS) score for a subject comprising:
(a) obtaining a liver biopsy sample from the subject;
(b) subjecting the sample to a multi-analyte profiling assay for gene expression of one or more genes, wherein the genes comprise ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, F9, or any combination thereof, to obtain a gene expression measurement for each of the one or more genes;
(c) normalizing the gene expression measurements of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to expression levels of a control set of genes to obtain a gene expression profile of the subject; and
(d) converting the gene expression profile of the subject into an FPS score, wherein the FPS score is a numerical value corresponding to a similarity between the gene expression profile of the subject and a high-risk reference gene expression profile or a low-risk reference gene expression profile.

39. The method of claim 38, wherein the subject is predicted to be at low risk for liver fibrosis progression if the FPS score is less than −1.3013.

40. The method of claim 38, wherein the subject is predicted to be at intermediate risk for liver fibrosis progression if the FPS score is between −1.3013 and +1.3013.

41. The method of claim 38, wherein the subject is predicted to be at high risk for liver fibrosis progression if the FPS score is greater than +1.3013.

42. The method of any one of claims 38 to 41, wherein the subject has or is suspected of having a disease, a condition, or a combination thereof that predisposes the subject to liver fibrosis.

43. The method of claim 42, wherein the disease, condition, or combination thereof that predisposes the subject to liver fibrosis comprises chronic infection of hepatitis B virus (HBV), chronic infection of hepatitis C virus (HCV), non-alcoholic fatty liver disease (NAFLD), non-alcoholic steatohepatitis (NASH), hereditary hemochromatosis, type 2 diabetes, obesity, tobacco use, alcohol abuse, long-term anabolic steroid use, tyrosinemia, alpha-1-antitrypsin deficiency, porphyria cutanea tarda, glycogen storage diseases, Wilson disease, or any combination thereof.

44. A diagnostic kit for determining a fibrosis progression signature (FPS) score of a subject comprising one or more reagents for use in a multi-analyte profiling assay.

45. The diagnostic kit of claim 44, wherein the one or more reagents for use in a multi-analyte profiling assay comprises one or more nucleic acid probes labeled with color-coded microbeads to mRNA transcribed from one or more genes selected from ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and F9.

46. A method of treating liver fibrosis in a subject at high risk for liver fibrosis progression, the method comprising:
(a) determining if the subject is at high risk for liver fibrosis progression by
(i) obtaining a liver biopsy sample from the subject;
(ii) subjecting the liver biopsy sample to a multi-analyte profiling assay for gene expression of one or more genes, wherein the genes comprise ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to obtain a gene expression measurement of each of the one or more genes;
(iii) normalizing the gene expression measurements of ANXA1, AEBP1, FBN1, IER3, CCL21, CXCR4, KRT7, IGFBP6, FILIP1L LOXL2, BCL2, SLC71, DDR1, NTS, PMM1, NAAA, TTR, PON3, HAAO, and/or F9 to expression levels of a control set of genes to obtain a gene expression profile of the subject;
(iv) converting the gene expression profile of the subject into an FPS score, wherein the FPS score is a numerical value corresponding to a similarity between the gene expression profile of the subject and a high-risk reference gene expression profile or a low-risk reference gene expression profile; and
(v) determining that the subject is at high risk for liver fibrosis progression if the FPS score is greater than +1.3013, and
(b) administering one or more treatments of liver fibrosis to the subject determined to be at high risk for developing liver fibrosis.

47. The method of claim 46, wherein the gene expression level of the one or more genes is determined by one or more methods selected the group consisting of microarrays, high-density expression array, DNA microarray, polymerase chain reaction (PCR), reverse transcriptase PCR (RT-PCR), real-time quantitative reverse transcription PCR (qRT-PCR), digital droplet PCR (ddPCR), serial analysis of gene expression (SAGE), Spotted cDNA arrays, GeneChip, spotted oligo arrays, bead arrays, RNA Seq, tiling array, northern blotting, hybridization microarray, in situ hybridization, or any combination thereof.

48. The method of any one of claim 46 or 47, wherein the one or more treatments of liver fibrosis comprises an anti-fibrotic therapy.

49. The method of claim 48, wherein the anti-fibrotic therapy comprises administration of one or more drugs to the subject, wherein the drugs are comprised of galunisertib, erlotinib, AM095, bortezomib, pioglitazone, metformin, epigallocatechin gallate (EGCG), I-BET 151, JQ1, captopril, and nizatidine (Selleck Chemicals); MG-132; or cenicriviroc.
